SEQUENCE LISTING (33 sequences)

SEQ ID NO: 1 - DNA, 2271 bp, Chlamydia trachomatis
  CDS (1)..(2271)
atggaaatcatggttcctcaaggaatttacgatggggagacgttaactgtatcatttccctatactgttataggagatccgagtgggactactgttttttctgcaggagagttaacattaaaaaatcttgacaattctattgcagctttgcctttaagttgttttgggaacttattagggagttttactgttttagggagaggacactcgttgactttcgagaacatacggacttctacaaatggggcagctctaagtaatagcgctgctgatggactgtttactattgagggttttaaagaattatccttttccaattgcaattcattacttgccgtactgcctgctgcaacgactaataagggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttgtagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctaagcaaccaacaagtggacaggcttctaatacgagtaataattacggagatggaggagctatcttctgtaagaatggtgcgcaagcaggatccaataactctggatcagtttcctttgatggagagggagtagttttctttagtagcaatgtagctgctgggaaagggggagctatttatgccaaaaagctctcggttgctaactgtggccctgtacaatttttaaggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccaaacttctaaaaattaacgatggtgaaggatacacaggggatattgtttttgctaatggaagcagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcagtcattggtagcacaactgctggttctgttacaattagtgggcctatcttttttgaggatttggatgatacagcttatgataggtatgattggctaggttctaatcaaaaaatcaatgtcctgaaattacagttagggactaagcccccagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttacc

SEQ ID NO: 2 - protein, 757 aa, Chlamydia trachomatis
MEIMVPQGIYDGETLTVSFPYTVIGDPSGTTVFSAGELTLKNLDNSIAALPLSCFGNLLGSFTVLGRGHSLTFENIRTSTNGAALSNSAADGLFTIEGFKELSFSNCNSLLAVLPAATTNKGSQTPTTTSTPSNGTIYSKTDLLLLNNEKFSFYSNLVSGDGGAIDAKSLTVQGISKLCVFQENTAQADGGACQVVTSFSAMANEAPIAFVANVAGVRGGGIAAVQDGQQGVSSSTSTEDPVVSFSRNTAVEFDGNVARVGGGIYSYGNVAFLNNGKTLFLNNVASPVYIAAKQPTSGQASNTSNNYGDGGAIFCKNGAQAGSNNSGSVSFDGEGVVFFSSNVAAGKGGAIYAKKLSVANCGPVQFLRNIANDGGAIYLGESGELSLSADYGDIIFDGNLKRTAKENAADVNGVTVSSQAISMGSGGKITTLRAKAGHQILFNDPIEMANGNNQPAQSSKLLKINDGEGYTGDIVFANGSSTLYQNVTIEQGRIVLREKAKLSVNSLSQTGGSLYMEAGSTLDFVTPQPPQQPPAANQLITLSNLHLSLSSLLANNAVTNPPTNPPAQDSHPAVIGSTTAGSVTISGPIFFEDLDDTAYDRYDWLGSNQKINVLKLQLGTKPPANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFT

SEQ ID NO: 3 - DNA, 2271 bp, Artificial Sequence
  Synthetic E. coli-codon optimized CT84
atggaaattatggttccgcagggtatctacgatggtgaaaccctgaccgtgtctttcccgtataccgttatcggtgatccgagcggtacgaccgttttcagcgccggtgaactgaccctgaaaaacctggataatagcattgcggcgctgccgctgtcttgcttcggtaacctgctgggttctttcaccgttctgggtcgtggccatagcctgacctttgaaaacattcgtaccagcaccaatggtgcggcgctgtctaatagcgcggcggatggtctgttcaccattgaaggtttcaaagaactgtctttctctaactgcaatagcctgctggcggttctgccggcggcgaccaccaacaaaggcagccagaccccgaccaccacgagcaccccgtctaacggcaccatctacagcaaaaccgatctgctgctgctgaacaacgaaaaattctctttttatagcaacctggtttctggtgatggtggtgcgattgatgcgaaaagcctgaccgttcagggtatctctaaactgtgcgttttccaggaaaacaccgcgcaggcggatggcggtgcgtgccaggttgttacctctttcagcgcgatggccaatgaagcgccgattgcgtttgttgccaacgtggcgggtgttcgtggtggtggtatcgcggcggtgcaggatggtcagcagggtgtgagctcttctacctctaccgaagatccggtggtgagcttcagccgtaacaccgcggtggaatttgatggtaacgtggcgcgcgttggtggtggtatctacagctacggtaacgtggcgttcctgaacaatggtaaaaccctgttcctgaataacgttgcgagcccggtgtatattgcggccaaacagccgacctctggtcaggcgtctaacaccagcaataactacggcgatggtggcgccattttctgcaaaaacggtgcgcaggcgggcagcaacaactctggcagcgtgagcttcgatggcgaaggcgtggtgtttttcagctctaatgtggcggcgggtaaaggcggcgcgatttatgcgaaaaaactgtctgttgcgaactgcggcccggtgcagttcctgcgtaacattgcgaacgatggtggtgcgatctacctgggtgaaagcggcgaactgtctctgagcgcggattatggcgatattatcttcgatggtaacattaaacgtaccgcgaaagaaaacgcggcggatgtgaacggtgtgaccgtgtcttctcaggcgattagcatgggtagcggcggcaaaattaccaccctgcgtgcgaaagcgggtcatcagatcctgttcaacgatccgatcgaaatggcgaacggtaataaccagccggcgcagtcttctaaactgctgaaaattaacgatggtgaaggttacaccggtgatattgtgttcgcgaacggttctagcaccctgtatcagaacgttaccatcgaacagggccgtatcgttctgcgtgaaaaagcgaaactgtctgttaacagcctgagccagaccggtggtagcctgtatatggaagcgggttctaccctggatttcgttaccccgcagccgccgcagcagccgccggcggcgaatcagctgatcaccctgagcaacctgcatctgtctctgtcttctctgctggcgaacaacgcggttaccaacccgccgaccaacccgccggcgcaggattctcatccggcggtgattggtagcaccaccgcgggtagcgttaccatttctggtccgattttctttgaagatctggatgataccgcgtacgatcgctacgattggctgggtagcaaccagaaaatcaacgttctgaaactgcaactgggcaccaaaccgccggcgaacgcgccgtctgatctgaccctgggtaacgaaatgccgaaatatggctaccagggttcttggaaactggcgtgggacccgaacaccgcgaacaacggtccgtacaccctgaaagcgacctggaccaaaaccggttacaatccgggcccggaacgtgttgcgtctctggttccgaactctctgtggggcagcattctggatattcgcagcgcgcattctgcgatccaggcgagcgtggatggtcgtagctattgccgcggtctgtgggttagcggtgtttctaacttcttctatcatgatcgcgatgcgctgggccagggctatcgctatattagcggtggttatagcctgggtgcgaacagctatttcggtagcagcatgttcggcctggcgttcacc

SEQ ID NO: 4 - DNA, 38 bp, Artificial Sequence
  Synthetic primer used to amplify the CT84 gene fragment
gggaattccatatggaaattatggttccgcagggtatc

SEQ ID NO: 5 - DNA, 35 bp, Artificial Sequence
  Synthetic primer used to amplify the CT84 gene fragment
ctagtctagattaggtgaacgccaggccgaacatg

SEQ ID NO: 6 - DNA, 1098 bp, Chlamydia trachomatis
  CDS (1)..(1092)
atgattttcgatgggaatattaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccaaacttctaaaaattaacgatggtgaaggatacacaggggatattgtttttgctaatggaagcagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcagtcattggtagcacaactgctggttctgttacaattagtgggcctatcttttttgaggatttggatgatacagcttatgataggtatgattggctaggttctaatcaaaaaatcaatgtcctgaaattacagttagggactaagcccccagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttacctaataa

SEQ ID NO: 7 - protein, 364 aa, Chlamydia trachomatis
MIFDGNIKRTAKENAADVNGVTVSSQAISMGSGGKITTLRAKAGHQILFNDPIEMANGNNQPAQSSKLLKINDGEGYTGDIVFANGSSTLYQNVTIEQGRIVLREKAKLSVNSLSQTGGSLYMEAGSTLDFVTPQPPQQPPAANQLITLSNLHLSLSSLLANNAVTNPPTNPPAQDSHPAVIGSTTAGSVTISGPIFFEDLDDTAYDRYDWLGSNQKINVLKLQLGTKPPANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFT

SEQ ID NO: 8 - DNA, 1542 bp, Chlamydia trachomatis
  CDS (1)..(1536)
atggctcaagctgatgggggagcttgtcaagtagtcaccagtttctctgctatggctaacgaggctcctattgcctttgtagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctaagcaaccaacaagtggacaggcttctaatacgagtaataattacggagatggaggagctatcttctgtaagaatggtgcgcaagcaggatccaataactctggatcagtttcctttgatggagagggagtagttttctttagtagcaatgtagctgctgggaaagggggagctatttatgccaaaaagctctcggttgctaactgtggccctgtacaatttttaaggaatatcgctaatgatggtggagcgatttatttaggagaatctggagagctcagtttatctgctgattatggagatattattttcgatgggaatattaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccaaacttctaaaaattaacgatggtgaaggatacacaggggatattgtttttgctaatggaagcagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcagtcattggtagcacaactgctggttctgttacaattagtgggcctatcttttttgaggatttggatgatacagcttatgataggtatgattggctaggttctaatcaaaaaatcaatgtcctgaaattacagttagggactaagcccccagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattaataa

SEQ ID NO: 9 - protein, 512 aa, Chlamydia trachomatis
MAQADGGACQVVTSFSAMANEAPIAFVANVAGVRGGGIAAVQDGQQGVSSSTSTEDPVVSFSRNTAVEFDGNVARVGGGIYSYGNVAFLNNGKTLFLNNVASPVYIAAKQPTSGQASNTSNNYGDGGAIFCKNGAQAGSNNSGSVSFDGEGVVFFSSNVAAGKGGAIYAKKLSVANCGPVQFLRNIANDGGAIYLGESGELSLSADYGDIIFDGNIKRTAKENAADVNGVTVSSQAISMGSGGKITTLRAKAGHQILFNDPIEMANGNNQPAQSSKLLKINDGEGYTGDIVFANGSSTLYQNVTIEQGRIVLREKAKLSVNSLSQTGGSLYMEAGSTLDFVTPQPPQQPPAANQLITLSNLHLSLSSLLANNAVTNPPTNPPAQDSHPAVIGSTTAGSVTISGPIFFEDLDDTAYDRYDWLGSNQKINVLKLQLGTKPPANAPSDLTLGNEMPKYGYQGSWKLAWDPNTANNGPYTLKATWTKTGYNPGPERVASLVPNSLWGSILDIRSAH

SEQ ID NO: 10 - DNA, 3639 bp, Artificial Sequence
  Synthetic version of PmpD
atggatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcggatactcttcccgggaaggtagagcaaagtactttgttctcagtaaccaatcccgtggttttccaaggtgtggaccaacaggatcaagtctcttcccaagggttaatttgtagttttacgagcagcaaccttgattctcctcgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggtggtgcttctgttttagggaccattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattactaataattccgaaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctgggaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagagcagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctagaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagccacattatgtagttatggttttaaacaagatgctggagctaagttggtattggctgctggagctaaactgaagattttagattcaggaactcctgtacaacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttattgaagaatgaggctaaagttccattgatgtctttcgttgcttctggtgatgaagcttcagccgaaatcagtaacttgtcggtttctgatttacagattcatgtagtaactccagagattgaagaagacacatacggccatatgggagattggtctgaggctaaaattcaagatggaactcttgtcattagttggaatcctactggatatcgattagatcctcaaaaagcaggggctttagtatttaatgcattatgggaagaaggggctgtcttgtctgctctgaaaaatgcacgctttgctcataatctcactgctcagcgtatggaataa

SEQ ID NO: 11 - protein, 1212 aa, Artificial Sequence
  Synthetic Construct
MDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRME

SEQ ID NO: 12 - DNA, 2109 bp, Artificial Sequence
  Synthetic version of PmpH
atggcgagctctcctcaagtgttaacacctaatgtaaccactccttttaagggggacgatgtttacttgaatggagactgcgcttttgtcaatgtctatgcaggggcagagaacggctcaattatctcagctaatggcgacaatttaacgattaccggacaaaaccatacattatcatttacagattctcaagggccagttcttcaaaattatgccttcatttcagcaggagagacacttactctgaaagatttttcgagtttgatgttctcgaaaaatgtttcttgcggagaaaagggaatgatctcagggaaaaccgtgagtatttccggagcaggcgaagtgattttttgggataactctgtggggtattctcctttgtctattgtgccagcatcgactccaactcctccagcaccagcaccagctcctgctgcttcaagctctttatctccaacagttagtgatgctcggaaagggtctattttttctgtagagactagtttggagatctcaggcgtcaaaaaaggggtcatgttcgataataatgccgggaattttggaacagtttttcgaggtaatagtaataataatgctggtagtgggggtagtgggtctgctacaacaccaagttttacagttaaaaactgtaaagggaaagtttctttcacagataacgtagcctcctgtggaggcggagtagtctacaaaggaactgtgcttttcaaagacaatgaaggaggcatattcttccgagggaacacagcatacgatgatttagggattcttgctgctactagtcgggatcagaatacggagacaggaggcggtggaggagttatttgctctccagatgattctgtaaagtttgaaggcaataaaggttctattgtttttgattacaactttgcaaaaggcagaggcggaagcatcctaacgaaagaattctctcttgtagcagatgattcggttgtctttagtaacaatacagcagaaaaaggcggtggagctatttatgctcctactatcgatataagcacgaatggaggatcgattctatttgaaagaaaccgagctgcagaaggaggcgccatctgcgtgagtgaagcaagctctggttcaactggaaatcttactttaagcgcttctgatggggatattgttttttctgggaatatgacgagtgatcgtcctggagagcgcagcgcagcaagaatcttaagtgatggaacgactgtttctttaaatgcttccggactatcgaagctgatcttttatgatcctgtagtacaaaataattcagcagcgggtgcatcgacaccatcaccatcttcttcttctatgcctggtgctgtcacgattaatcagtccggtaatggatctgtgatttttaccgccgagtcattgactccttcagaaaaacttcaagttcttaactctacttctaacttcccaggagctctgactgtgtcaggaggggagttggttgtgacggaaggagctaccttaactactgggaccattacagccacctctggacgagtgactttaggatccggagcttcgttgtctgccgttgcaggtgctgcaaataataattatacttgtacagtatctaagttggggattgatttagaatcctttttaactcctaactataagacggccatactgggtgcggatggaacagttactgttaacagcggctctactttagacctagtgatggagagtgaggcagaggtatatgataatccgctttttgtgggatcgctgacaattccttttgttactctatcttctagtagtgctagtaacggagttacaaaaaattctgtcactattaatgatgcagacgctgcgcactatgggtatcaaggctcttggtctgcagattggacgaaaccgcctctggctcctgatgctaaggggatggtacctcctaataccaataacactctgtatctgacatggagacctgcttcgaattacggtgaatatcgactggatcctcagagaaagggagaactagtacccaactctctttgggtagcgggatctgcattaagaacctttactaatggtttgaaagaacactatgtttcttaa

SEQ ID NO: 13 - protein, 702 aa, Artificial Sequence
  Synthetic Construct
MASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVS

SEQ ID NO: 14 - DNA, 1749 bp, Artificial Sequence
  Synthetic version of PmpI
atgctctttggccaggatcccttaggtgaaaccgccctcctcactaaaaatcctaatcatgtcgtctgtacattttttgaggactgtaccatggagagcctctttcctgctctttgtgctcatgcatcacaagatgatcctttgtatgtacttggaaattcctactgttggttcgtatctaaactccatatcacggaccccaaagaggctctttttaaagaaaaaggagatctttccattcaaaattttcgcttcctttccttcacagattgctcttccaaggaaagctctccttctattattcatcaaaagaatggtcagttatccttgcgcaataatggtagcatgagtttctgtcgaaatcatgctgaaggctctggaggagccatctctgcggatgccttttctctacaacacaactatcttttcacagcttttgaagagaattcttctaaaggaaatggcggagccattcaggctcaaaccttctctttatctagaaatgtgtcgcctatttctttcgcccgtaatcgtgcggatttaaatggcggcgctatttgctgtagtaatcttatttgttcagggaatgtaaaccctctctttttcactggaaactccgccacgaatggaggcgctatttgttgtatcagcgatctaaacacctcagaaaaaggctctctctctcttgcttgtaaccaagaaacgctatttgcaagcaattctgctaaagaaaaaggcggggctatttatgccaagcacatggtattgcgttataacggtcctgtttccttcattaacaacagcgctaaaataggtggagctatcgccatccagtccggagggagtctctctatccttgcaggtgaaggatctgttctgttccagaataactcccaacgcacctccgaccaaggtctagtaagaaacgccatctacttagagaaagatgcgattctttcttccttagaagctcgcaacggagatattcttttctttgatcctattgtacaagaaagtagcagcaaagaatcgcctcttccctcctctttgcaagccagcgtgacttctcccaccccagccaccgcatctcctttagttattcagacaagtgcaaaccgttcagtgattttctcgagcgaacgtctttctgaagaagaaaaaactcctgataacctcacttcccaactacagcagcctatcgaactgaaatccggacgcttagttttaaaagatcgcgctgtcctttccgcgccttctctctctcaggatcctcaagctctcctcattatggaagcgggaacttctttaaaaacttcctctgatttgaagttagctacgctaagtattccccttcattccttagatactgaaaaaagcgtaactatccacgcccctaacctttctatccaaaagatcttcctctctaattctggagatgagaatttttatgaaaatgtagagcttctcagtaaagagcaaaacaatattcctctccttactctctctaaagagcaatctcatttacatcttcctgatgggaacctctcttctcactttggatatcaaggagattggactttttcttggaaagattctgatgaagggcattctctgattgctaattggacgcctaaaaactatgtgcctcatccagaacgtcaatctacactcgttgcgaacactctttggaacacctattccgatatgcaagctgtgcagtcgatgattaatacaatagcgcacggatag

SEQ ID NO: 15 - protein, 582 aa, Artificial Sequence
  Synthetic Construct
MLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHG

SEQ ID NO: 16 - protein, 4 aa, Artificial Sequence
  Synthetic heterologous polypeptide
AWWP

SEQ ID NO: 17 - DNA, 37 bp, Artificial Sequence
  Synthetic primer used to amplify the CT40 gene fragment
cgactagtttattaggtaaatgctagaccaaacatcg

SEQ ID NO: 18 - DNA, 1554 bp, Artificial Sequence
  Synthetic version of OmcB-1
atggagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 19 - protein, 517 aa, Artificial Sequence
  Synthetic Construct
MESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 20 - DNA, 465 bp, Artificial Sequence
  Synthetic version of OmpH-1
atgaattctacaggcacaattggaatcgttaatttacgtcgctgcctagaagagtctgctcttgggaaaaaagaatctgctgaattcgaaaagatgaaaaaccaattctctaacagcatggggaagatggaggaagaactgtcttctatctattccaagctccaagacgacgattacatggaaggtctatccgagaccgcagctgccgaattaagaaaaaaattcgaagatctatctgcagaatacaacacagctcaagggcagtattaccaaatattaaaccaaagtaatctcaagcgcatgcaaaagattatggaagaagtgaaaaaagcttctgaaactgtgcgtattcaagaaggcttgtcagtccttcttaacgaagatattgtcttatctatcgatagttcggcagataaaaccgatgctgttattaaagttcttgatgattcttttcaaaataattaa

SEQ ID NO: 21 - protein, 154 aa, Artificial Sequence
  Synthetic Construct
MNSTGTIGIVNLRRCLEESALGKKESAEFEKMKNQFSNSMGKMEEELSSIYSKLQDDDYMEGLSETAAAELRKKFEDLSAEYNTAQGQYYQILNQSNLKRMQKIMEEVKKASETVRIQEGLSVLLNEDIVLSIDSSADKTDAVIKVLDDSFQNN

SEQ ID NO: 22 - DNA, 12 bp, Artificial Sequence
  Synthetic primer used to clone into the pET-43.1 Ek/LIC
gacgacgacaag

SEQ ID NO: 23 - DNA, 15 bp, Artificial Sequence
  Synthetic primer used to clone into the pET-43.1 Ek/LIC
gaggagaagcccggt

SEQ ID NO: 24 - DNA, 42 bp, Artificial Sequence
  Synthetic primer used to amplify CT84 gene fragment
gggaattcccatatggaaatcatggttcctcaaggaatttac

SEQ ID NO: 25 - DNA, 37 bp, Artificial Sequence; identical to SEQ ID NO: 17
  Synthetic primer used to amplify CT84 gene fragment

SEQ ID NO: 26 - DNA, 40 bp, Artificial Sequence
  Synthetic primer used to amplify CT57 gene fragment
gggaattcccatatggctcaagctgatgggggagcttgtc

SEQ ID NO: 27 - DNA, 40 bp, Artificial Sequence
  Synthetic primer used to amplify CT57 gene fragment
cgactagtttattaatgcgcagatcgtatatctaaaatgg

SEQ ID NO: 28 - DNA, 45 bp, Artificial Sequence
  Synthetic primer used to amplify the CT40 gene fragment
gggaattcccatatgattttcgatgggaatattaaaagaacagcc

SEQ ID NO: 29 - protein, 973 aa, Chlamydia pneumoniae
MKTSIRKFLISTTLAPCFASTAFTVEVIMPSENFDGSSGKIFPYTTLSDPRGTLCIFSGDLYIANLDNAISRTSSSCFSNRAGALQILGKGGVFSFLNIRSSADGAAISSVITQNPELCPLSFSGFSQMIFDNCESLTSDTSASNVIPHASAIYATTPMLFTNNDSILFQYNRSAGFGAAIRGTSITIENTKKSLLFNGNGSISNGGALTGSAAINLINNSAPVIFSTNATGIYGGAIYLTGGSMLTSGNLSGVLFVNNSSRSGGAIYANGNVTFSNNSDLTFQNNTASPQNSLPAPTPPPTPPAVTPLLGYGGAIFCTPPATPPPTGVSLTISGENSVTFLENIASEQGGALYGKKISIDSNKSTIFLGNTAGKGGAIAIPESGELSLSANQGDILFNKNLSITSGTPTRNSIHFGKDAKFATLGATQGYTLYFYDPITSDDLSAASAAATVVVNPKASADGAYSGTIVFSGETLTATEAATPANATSTLNQKLELEGGTLALRNGATLNVHNFTQDEKSVVIMDAGTTLATTNGANNTDGAITLNKLVINLDSLDGTKAAVVNVQSTNGALTISGTLGLVKNSQDCCDNHGMFNKDLQQVPILELKATSNTVTTTDFSLGTNGYQQSPYGYQGTWEFTIDTTTHTVTGNWKKTGYLPHPERLAPLIPNSLWANVIDLRAVSQASAADGEDVPGKQLSITGITNFFHANHTGDARSYRHMGGGYLINTYTRITPDAALSLGFGQLFTKSKDYLVGHGHSNVYFATVYSNITKSLFGSSRFFSGGTSRVTYSRSNEKVKTSYTKLPKGRCSWSNNCWLGELEGNLPITLSSRILNLKQIIPFVKAEVAYATHGGIQENTPEGRIFGHGHLLNVAVPVGVRFGKNSHNRPDFYTIIVAYAPDVYRHNPDCDTTLPINGATWTSIGNNLTRSTLLVQASSHTSVNDVLEIFGHCGCDIRRTSRQYTLDIGSKLRF

SEQ ID NO: 30 - DNA, 2769 bp, Chlamydia pneumoniae
atgcgattttcgctctgcggatttcctctagttttttcttttacattgctctcagtcttcgacacttctttgagtgctactacgatttctttaaccccagaagatagttttcatggagatagtcagaatgcagaacgttcttataatgttcaagctggggatgtctatagccttactggtgatgtctcaatatctaacgtcgataactctgcattaaataaagcctgcttcaatgtgacctcaggaagtgtgacgttcgcaggaaatcatcatgggttatattttaataatatttcctcaggaactacaaaggaaggggctgtactttgttgccaagatcctcaagcaacggcacgtttttctgggttctccacgctctcttttattcagagccccggagatattaaagaacagggatgtctctattcaaaaaatgcacttatgctcttaaacaattatgtagtgcgttttgaacaaaaccaaagtaagactaaaggcggagctattagtggggcgaatgttactatagtaggcaactacgattccgtctctttctatcagaatgcagccacttttggaggtgctatccattcttcaggtcccctacagattgcagtaaatcaggcagagataagatttgcacaaaatactgccaagaatggttctggaggggctttgtactccgatggtgatattgatattgatcagaatgcttatgttctatttcgagaaaatgaggcattgactactgctataggtaagggaggggctgtctgttgtcttcccacttcaggaagtagtactccagttcctattgtgactttctctgacaataaacagttagtctttgaaagaaaccattccataatgggtggcggagccatttatgctaggaaacttagcatctcttcaggaggtcctactctatttatcaataatatatcatatgcaaattcgcaaaatttaggtggagctattgccattgatactggaggggagatcagtttatcagcagagaaaggaacaattacattccaaggaaaccggacgagcttaccgtttttgaatggcatccatcttttacaaaatgctaaattcctgaaattacaggcgagaaatggatactctatagaattttatgatcctattacttctgaagcagatgggtctacccaattgaatatcaacggagatcctaaaaataaagagtacacagggaccatactcttttctggagaaaagagtctagcaaacgatcctagggattttaaatctacaatccctcagaacgtcaacctgtctgcaggatacttagttattaaagagggggccgaagtcacagtttcaaaattcacgcagtctccaggatcgcatttagttttagatttaggaaccaaactgatagcctctaaggaagacattgccatcacaggcctcgcgatagatatagatagcttaagctcatcctcaacagcagctgttattaaagcaaacaccgcaaataaacagatatccgtgacggactctatagaacttatctcgcctactggcaatgcctatgaagatctcagaatgagaaattcacagacgttccctctgctctctttagagcctggagccgggggtagtgtgactgtaactgctggagatttcctaccggtaagtccccattatggttttcaaggcaattggaaattagcttggacaggaactggaaacaaagttggagaattcttctgggataaaataaattataagcctagacctgaaaaagaaggaaatttagttcctaatatcttgtgggggaatgctgtagatgtcagatccttaatgcaggttcaagagacccatgcatcgagcttacagacagatcgagggctgtggatcgatggaattgggaatttcttccatgtatctgcctccgaagacaatataaggtaccgtcataacagcggtggatatgttctatctgtaaataatgagatcacacctaagcactatacttcgatggcattttcccaactctttagtagagacaaggactatgcggtttccaacaacgaatacagaatgtatttaggatcgtatctctatcaatatacaacctccctagggaatattttccgttatgcttcgcgtaaccctaatgtaaacgtcgggattctctcaagaaggtttcttcaaaatcctcttatgatttttcattttttgtgtgcttatggtcatgccaccaatgatatgaaaacagactacgcaaatttccctatggtgaaaaacagctggagaaacaattgttgggctatagagtgcggagggagcatgcctctattggtatttgagaacggaagacttttccaaggtgccatcccatttatgaaactacaattagtttatgcttatcagggagatttcaaagagacgactgcagatggccgtagatttagtaatgggagtttaacatcgatttctgtacctctaggcatacgctttgagaagctggcactttctcaggatgtactctatgactttagtttctcctatattcctgatattttccgtaaggatccctcatgtgaagctgctctggtgattagcggagactcttggcttgttccggcagcacacgtatcaagacatgcttttgtagggagtggaacgggtcggtatcactttaacgactatactgagctcttatgtcgaggaagtatagaatgccgcccccatgctaggaattataatataaactgtggaagcaaatttcgtttttag

SEQ ID NO: 31 - DNA, 2769 bp, Chlamydia pneumoniae
atgcgattttcgctctgcggatttcctctagttttttcttttacattgctctcagtcttcgacacttctttgagtgctactacgatttctttaaccccagaagatagttttcatggagatagtcagaatgcagaacgttcttataatgttcaagctggggatgtctatagccttactggtgatgtctcaatatctaacgtcgataactctgcattaaataaagcctgcttcaatgtgacctcaggaagtgtgacgttcgcaggaaatcatcatgggttatattttaataatatttcctcaggaactacaaaggaaggggctgtactttgttgccaagatcctcaagcaacggcacgtttttctgggttctccacgctctcttttattcagagccccggagatattaaagaacagggatgtctctattcaaaaaatgcacttatgctcttaaacaattatgtagtgcgttttgaacaaaaccaaagtaagactaaaggcggagctattagtggggcgaatgttactatagtaggcaactacgattccgtctctttctatcagaatgcagccacttttggaggtgctatccattcttcaggtcccctacagattgcagtaaatcaggcagagataagatttgcacaaaatactgccaagaatggttctggaggggctttgtactccgatggtgatattgatattgatcagaatgcttatgttctatttcgagaaaatgaggcattgactactgctataggtaagggaggggctgtctgttgtcttcccacttcaggaagtagtactccagttcctattgtgactttctctgacaataaacagttagtctttgaaagaaaccattccataatgggtggcggagccatttatgctaggaaacttagcatctcttcaggaggtcctactctatttatcaataatatatcatatgcaaattcgcaaaatttaggtggagctattgccattgatactggaggggagatcagtttatcagcagagaaaggaacaattacattccaaggaaaccggacgagcttaccgtttttgaatggcatccatcttttacaaaatgctaaattcctgaaattacaggcgagaaatggatactctatagaattttatgatcctattacttctgaagcagatgggtctacccaattgaatatcaacggagatcctaaaaataaagagtacacagggaccatactcttttctggagaaaagagtctagcaaacgatcctagggattttaaatctacaatccctcagaacgtcaacctgtctgcaggatacttagttattaaagagggggccgaagtcacagtttcaaaattcacgcagtctccaggatcgcatttagttttagatttaggaaccaaactgatagcctctaaggaagacattgccatcacaggcctcgcgatagatatagatagcttaagctcatcctcaacagcagctgttattaaagcaaacaccgcaaataaacagatatccgtgacggactctatagaacttatctcgcctactggcaatgcctatgaagatctcagaatgagaaattcacagacgttccctctgctctctttagagcctggagccgggggtagtgtgactgtaactgctggagatttcctaccggtaagtccccattatggttttcaaggcaattggaaattagcttggacaggaactggaaacaaagttggagaattcttctgggataaaataaattataagcctagacctgaaaaagaaggaaatttagttcctaatatcttgtgggggaatgctgtagatgtcagatccttaatgcaggttcaagagacccatgcatcgagcttacagacagatcgagggctgtggatcgatggaattgggaatttcttccatgtatctgcctccgaagacaatataaggtaccgtcataacagcggtggatatgttctatctgtaaataatgagatcacacctaagcactatacttcgatggcattttcccaactctttagtagagacaaggactatgcggtttccaacaacgaatacagaatgtatttaggatcgtatctctatcaatatacaacctccctagggaatattttccgttatgcttcgcgtaaccctaatgtaaacgtcgggattctctcaagaaggtttcttcaaaatcctcttatgatttttcattttttgtgtgcttatggtcatgccaccaatgatatgaaaacagactacgcaaatttccctatggtgaaaaacagctggagaaacaattgttgggctatagagtgcggagggagcatgcctctattggtatttgagaacggaagacttttccaaggtgccatcccatttatgaaactacaattagtttatgcttatcagggagatttcaaagagacgactgcagatggccgtagatttagtaatgggagtttaacatcgatttctgtacctctaggcatacgctttgagaagctggcactttctcaggatgtactctatgactttagtttctcctatattcctgatattttccgtaaggatccctcatgtgaagctgctctggtgattagcggagactcctggcttgttccggcagcacacgtatcaagacatgcttttgtagggagtggaacgggtcggtatcactttaacgactatactgagctcttatgtcgaggaagtatagaatgccgcccccatgctaggaattataatataaactgtggaagcaaatttcgtttttag

SEQ ID NO: 32 - protein, 995 aa, Chlamydia pneumoniae
MYLFFYSLSLICRIIWFHLYVQMKTSIRKFLISTTLAPCFASTAFTVEVIMPSENFDGSSGKIFPYTTLSDPRGTLCIFSGDLYIANLDNAISRTSSSCFSNRAGALQILGKGGVFSFLNIRSSADGAAISSVITQNPELCPLSFSGFSQMIFDNCESLTSDTSASNVIPHASAIYATTPMLFTNNDSILFQYNRSAGFGAAIRGTSITIENTKKSLLFNGNGSISNGGALTGSAAINLINNSAPVIFSTNATGIYGGAIYLTGGSMLTSGNLSGVLFVNNSSRSGGAIYANGNVTFSNNSDLTFQNNTASPQNSLPAPTPPPTPPAVTPLLGYGGAIFCTPPATPPPTGVSLTISGENSVTFLENIASEQGGALYGKKISIDSNKSTIFLGNTAGKGGAIAIPESGELSLSANQGDILFNKNLSITSGTPTRNSIHFGKDAKFATLGATQGYTLYFYDPITSDDLSAASAAATVVVNPKASADGAYSGTIVFSGETLTATEAATPANATSTLNQKLELEGGTLALRNGATLNVHNFTQDEKSVVIMDAGTTLATTNGANNTDGAITLNKLVINLDSLDGTKAAVVNVQSTNGALTISGTLGLVKNSQDCCDNHGMFNKDLQQVPILELKATSNTVTTTDFSLGTNGYQQSPYGYQGTWEFTIDTTTHTVTGNWKKTGYLPHPERLAPLIPNSLWANVIDLRAVSQASAADGEDVPGKQLSITGITNFFHANHTGDARSYRHMGGGYLINTYTRITPDAALSLGFGQLFTKSKDYLVGHGHSNVYFATVYSNITKSLFGSSRFFSGGTSRVTYSRSNEKVKTSYTKLPKGRCSWSNNCWLGELEGNLPITLSSRILNLKQIIPFVKAEVAYATHGGIQENTPEGRIFGHGHLLNVAVPVGVRFGKNSHNRPDFYTIIVAYAPDVYRHNPDCDTTLPINGATWTSIGNNLTRSTLLVQASSHTSVNDVLEIFGHCGCDIRRTSRQYTLDIGSKLRF

SEQ ID NO: 33 - DNA, 2988 bp, Chlamydia pneumoniae
ttaaaatcgtaatttgcttcctatatctagagtatattgacgggaggttctgcgaatatcacatccacagtgcccgaagatctctagaacatcatttactgaagtatggctggatgcttgtactagcaaagtacttctggttagattattccctatagaggtccacgtagctccattaataggtaatgtcgtatcgcaatcaggattgtgacgatagacatcaggagcataggctacgattatagtgtaaaaatctggtcgattatgagaatttttaccaaagcggacgcctacgggaactgcaacgttgagtagatgaccgtgtccaaaaatcctcccctcaggggtattttcttggatgcccccatgagtcgcgtaagcaacttcagcttttacaaagggaatgatctgcttgaggtttaagatgcgagaagagagagtgatgggaaggttcccttcgagttctcctaaccagcaattgttactccaagagcagcgccctttaggcaattttgtatatgaagtctttactttctcattgctacggctataggtaactcgagaagtgcctcctgagaagaatctcgatgatccaaacagagacttggtgatgttagagtatactgtagcgaaataaacgttagaatgaccgtgacctacgaggtaatccttagattttgtaaacagctgtccaaaacctagacttaacgcagcatctggagtgatgcgtgtgtaggtattgatgaggtagcctccacccatatggcggtagctgcgtgcatcaccggtatgattcgcatggaagaaatttgtaattcctgtgatgctcagttgcttcccagggacatcttcgccatcagctgctgacgcttgacttacagctcgtaaatctatgacgtttgcccataggctattaggaatgaggggagcaagacgctccggatgaggaagataaccggtttttttccaatttcctgtgaccgtatgggttgtcgtgtctatggtaaactcccaagttccttgatacccatagggagattgctgatagccgtttgtgccgagactgaagtccgtagtggttacagtatttgaagtcgctttgagttctaaaatcggaacttgctgtaaatctttattaaacatcccgtggttgtcacagcaatcttgagagtttttcacaagtcctaaagttccggatatagtgagagctccattggtactctgcacattaacgacagccgctttagtgccatccaaagaatccagattgattacaagcttgtttaaggtgatagcaccgtcagtattattagctccatttgtagttgctaatgtggtccctgcatccatgatgacgacggacttttcatcttgcgtgaagttatgaacatttaaggtagcaccgtttcttaaagcgagagtaccgccttcaagttctagcttttggtttaatgtagatgtagcatttgcaggggttgctgcttcggtagcagtgagggtttctcctgaaaagacaatagtccctgaatacgcaccatctgcactggctttgggattgacgaccacagtagcggctgcggatgcagcagataaatcatcagatgtaatcggatcatagaagtatagggtatagccttgcgtagctcctagagtggcaaacttggcatcttttccgaagtgaatactattgcgagtaggtgtcccactagtgatgctgaggttcttgttaaagaggatatcaccttgatttgcggatagagagagctccccagattcgggaatagcaatagcgcctccttttccagctgtatttccaagaaatattgtagatttattagaatctatagagatctttttgccatagagggctcctccttgttcggaggcaatgttttctaggaatgtaacgctgttttctccagatatagtcaggctaacacctgttggtgggggggtagctggaggagtacagaagatggcgcctccatatcctaacaaaggagtgactgctggtggtgtaggtggaggtgtaggtgcaggtaaggagttttgtggagatgctgtattgttttggaaagtcaggtcgctgttattagaaaatgtgacatttccgttagcatagatagcgcctcctgagcgcgagctattattaacgaacaagactcctgagaggttcccagaggtgagcatagatcctccggtaaggtaaatagccccaccatagatccctgtagcattcgttgagaaaatcacaggagcgctattgttgatgaggttgatcgctgcagatcccgtgagggcccctccattagagatggatccattaccattaaagagaaggctctttttcgtattttctattgtgatgcttgtgcctcgaatggcagctccaaatcctgcagaacggttgtattggaatagtatggagtcattgtttgtaaagagcatgggcgttgtagcgtaaatcgccgatgcgtgaggtatgacattactcgctgaggtatctgaagtcaaagattcacagttatcgaagatcatctgactaaatcctgaaaaactcaagggacatagttcaggattttgggtgattacactactaatcgcggctccgtcagctgaagaacggatatttaagaaggagaaaaccccaccttttcctaagatttgtagtgctcccgccctattgctaaagcaactggaagaggttctggatatggcattatcaagattcgcaatgtagagatcccctgaaaaaatacagagtgtccctctaggatcagaaagtgttgtgtaaggaaaaatcttcccactcgatccatcaaagttctcggaaggcatgataacttctacagtaaacgctgttgaagcaaaacatggcgccagtgtggtagaaattaagaacttacgaatagacgttttcatttgcacgtagagatgaaaccagattatcctacaaataagggaaaggctgtaaaaaaacaagtacaa

(19) United States
(12) Patent Application Publication
(10) Pub. No.: US 2010/0310593 A1
(43) Pub. Date: Dec. 9, 2010

(54) Vaccines Against Chlamydia Infection

(75) Inventors: So-ching Brazer, Leesburg, VA (US); James Adam Crawford, Dublin, OH (US); Chunghee Lee, Clarksburg, MD (US); Sukjoon Park, Germantown, MD (US); W. James Jackson, Marriottsville, MD (US); Michael Joseph Lacy, Damascus, MD (US); Hang Lu, Gaithersburg, MD (US)

Correspondence Address: Sterne, Kessler, Goldstein & Fox, P.L.L.C., 1100 New York Avenue, NW, Washington, DC, 20005, US

(73) Assignee: EMERGENT PRODUCT DEVELOPMENT GAITHERSBURG INC., Gaithersburg, MD (US)

(21) Appl. No.: 12/664,554

(22) PCT Filed: Jun. 16, 2008
(86) PCT No.: PCT/US2008/007490

§ 371 Date: Aug. 20, 2010

Related U.S. Application Data
(60) Provisional application No. 60/929,129, filed on Jun. 14, 2007.

(51) Int. Cl. A61K 39/02; C07H 21/04; C12N 15/63; C12N 1/21; C07K 14/295; A61K 48/00; A61K 38/16; A61P 31/04

(52) U.S. Cl. 424/190.1; 536/23.7; 435/320.1; 435/252.33; 530/350; 514/44 R; 514/21.2

(57) ABSTRACT

The present invention is directed to providing a vaccine to enhance the immune response of an animal in need of protection against a Chlamydia infection. The present invention is also directed toward an isolated nucleic acid encoding a polypeptide comprising at least 70% identity to any one of SEQ ID NOS: 2, 11, 13, 19, or 21, wherein the polypeptide is soluble in the absence of denaturing agents. In some aspects of the invention, the polynucleotide is codon-optimized. In some embodiments, the present invention is related to the polypeptide encoded by the polynucleotide of the invention. Administration of polypeptides of the present invention can be used as a method to treat or prevent a Chlamydia infection in an animal in need thereof.

### FIELD OF THE INVENTION

The present invention is directed to a pharmaceutical composition and a vaccine useful for the treatment and prevention of a Chlamydia infection and conditions related to a Chlamydia infection. The present invention includes soluble, recombinant PmpG, PmpD, PmpH, PmpI, OmcB and OmpH polypeptides that are immunogenic when administered to a subject. In one aspect of the invention, recombinant PmpG, PmpD, PmpH, and PmpI polypeptides lack an N-terminus signal sequence and a hydrophobic C-terminal transmembrane domain. In another aspect of the invention, recombinant OmcB and OmpH polypeptides lack an N-terminal signal sequence.

### BACKGROUND OF THE INVENTION

### Background Art

Chlamydia is a genus of gram-negative bacteria, obligate intracellular parasites of eukaryotic cells. Species of the Chlamydia genus include, but are not limited to Chlamydia psittaci, Chlamydia pecorum, Chlamydia pneumoniae, and Chlamydia trachomatis. The Chlamydia genus can cause a variety of diseases in humans, mammals, and birds; the most notable diseases in humans being trachoma, the leading cause of preventable blindness worldwide, urogenital infections and psittacosis. Other conditions caused by Chlamydia include a variety of sexually transmitted diseases such as lymphogranuloma venereum, urethritis, cervicitis, endometritis, and salpingitis (Thylefors et al., Bull W.H.O. 73:115-121 (1995)). Generally, Chlamydia trachomatis is considered the world's most common sexually transmitted bacterial pathogen. An estimated 400 million people have active infectious trachoma, while 90 million have a sexually transmitted disease caused by C. trachomatis. Diagnosis and detection of this organism is often on the basis of the pathologic or clinical findings and may be confirmed by isolation and staining techniques.

Generally, Chlamydia exhibit morphologic and structural similarities to gram-negative bacteria including a trilaminar outer membrane, which contains lipopolysaccharide and several membrane proteins that are structurally and functionally analogous to proteins found in Escherichia coli. However, Chlamydia contains a unique biphasic life cycle consisting of production of a metabolically inactive but infectious elemental bodies (EB), and production of a replicating but non-infectious reticular bodies (RB) during the intracellular stage. The replicative stage of the life-cycle takes place within a membrane-bound inclusion which sequesters the bacteria away from the cytoplasm of the infected host cell. The reticular bodies, after multiplication by binary fission, are transformed into elemental bodies which come out of the host cell and infect new cells. The outer membrane proteins of EB are highly cross-linked with disulfide bonds. The Chlamydial outer membrane complex (COMC), which includes the major outer membrane protein (MOMP or OmpA), is a major component of the Chlamydial outer membrane and is made up of a number of cysteine-rich proteins (Everett et al., J. Bacteriol. 177:877-882 (1995); Newhall et al., Infect. Immun. 55:162-168 (1986); Sardinia et al., J. Gen. Microbiol. 134:997-1004 (1988)). The COMC is present on the outer membrane proteins of EB, but not of RB. In contrast, MOMP is present throughout the developmental cycle in both EB and RB and is thought to have a structural role due to its predominance and extensive disulfide crosslinking in the EB membrane. Another function of MOMP is as a porin which allows for non-specific diffusion of small molecules into Chlamydia (Bavoil et al., Infect. Immun. 44:479-485 (1984); Wyllie et al. Infect. Immun. 66:5202-5207 (1998)).

Chlamydial infections often have no overt symptoms, so irreversible damage can be done before the patient is aware of the infection. Treatment with antimicrobial drugs can generally be used once an invention is diagnosed. However, treatment of Chlamydia with existing antimicrobial drugs may lead to development of drug resistant bacterial strains, particularly where the patient is concurrently infected with other common bacterial infections. Thus, prevention of the infection via a vaccine is considered the best way to protect from the damage caused by Chlamydia. Development and production of effective Chlamydial vaccines is an important public health priority.

As with many pathogens, the development of a vaccine to Chlamydia has proven difficult. However, studies with C. trachomatis have indicated that safe and effective vaccine against Chlamydia may be attainable. For example, mice which have recovered from a lung infection with C. trachomatis were protected from infertility induced by a subsequent vaginal challenge (Pal et al., Infect. Immun. 64:5341 (1996)). Protection from Chlamydial infections has been associated with Th1 immune responses, particularly the induction of INFγ-producing CD4+T-cells (Igietsemes et al., Immunology 5:317 (1993)). The adoptive transfer of CD4+ cell lines or clones to nude or SCID mice conferred protection from challenge or cleared chronic disease (Igietseme et al., Regional Immunology 5:317 (1993); Magee et al., Regional Immunology 5:305 (1993)), and in vivo depletion of CD4+T cells exacerbated disease post-challenge (Landers et al., Infect. Immun. 59:3774 (1991); Magee et al., Infect. Immun. 63:516 (1995)). It has also been shown that passive transfer of high-titer antichlamydial sera significantly reduced chlamydial shedding in guinea pigs, (Rank and Batteiger, Infect. Immun. 57:299-301 (1989) and the presence of sufficiently high titers of neutralizing antibody produced by implanted hybridoma tumors producing IgG and IgA MAbs specific for MOMP also exerted a protective effect (Cotter et al., Infect. Immun. 63:4704 (1995)).

Much of the focus for a vaccine candidate has been on the Chlamydial major outer membrane protein (MOMP) (see, e.g., U.S. Pat. Nos. 5,770,714 and 5,821,055; and PCT Pub. Appl. Nos. WO 98/10789; WO 99/10005; WO 97/41889; WO 98/02546; WO 94/06827; and WO 96/31236. It is estimated that MOMP makes up over 60% of the total outer membrane of Chlamydia (Caldwell et al., Infect. Immun. 31:1161-1176 (1981)). The exposed surface antigens on MOMP confer varying serotype, serogroup and species reactivities (Stephens et al., J. Exp. Med. 167:817-831 (1988)). The protein consists of five conserved segments and four variable segments with the variable segments corresponding to surface exposed regions and conferring serologic specificity (Stephens et al., J. Exp. Med. 167:817-831 (1988)). It has been suggested that these variable segments provide Chlamydia with antigenic variation, which in turn is important in evading the host immune response (Stephens, Antigenic Variation of Chlamydia trachomatis, p. 51-62. In J. W. Moulder (ed.), Intracellular Parasitism. CRC Press, Boca Raton (1989)). A potential problem in making a vaccine to an antigenically variant region is that a vaccine to one region of MOMP may only confer protection to that serovar. Also, making a subunit vaccine to an antigenic variable region may prove difficult since conformational antigenic determinants may be essential to elicit effective immunization (Fan et al., J. Infect. Dis. 176:713-721 (1997)). Also, data by Williams et al. (Infect. Immun. 45:674-678 (1984); Infect. Immun. 65:2876-2882 (1997)), both incorporated herein by reference, suggests that an antibody against one Chlamydia protein could play a partial protective role but not a complete role for immunity, even though the antibodies neutralize infectivity in vitro. These difficulties suggest that other vaccine targets should be explored.

Recently, another Chlamydial outer membrane protein, CT110 (also referred to as HMW or PmpG), has been identified as a potential vaccine candidate (see, e.g., U.S. Pat. Nos. 6,887,843 and 6,642,023). While highly immunogenic, CT110 was calculated to have a transmembrane region and localize to the outer membrane. Transmembrane proteins generally have poor solubility in an aqueous environment in the absence of detergents. Consequently, vaccines utilizing transmembrane proteins are often more difficult and more costly to manufacture than fully secreted or intracellular proteins.

In addition to MOMP and CT110, other outer membrane proteins are known and thus could act as potential immunogenic targets for vaccines. These outermembrane proteins include other members of the Pmp family, e.g., PmpD, PmpH, PmpI, as well as the outermembrane proteins OmcB and OmpH. As with CT110, these proteins are also transmembrane proteins and thus are expected to provide the same solubility and purification difficulties associated with CT110.

There is a need in the field for the development and efficient production of vaccines that provide protection against Chlamydia.

### BRIEF SUMMARY OF THE INVENTION

The present invention is directed to providing a vaccine to enhance the immune response of an animal in need of protection against a Chlamydia infection. The present invention is also directed toward an isolated nucleic acid encoding a polypeptide with at least about 70%, 75%, 80%, 85%, 90%, 95% or even 99% sequence identity to any one of SEQ ID NOS: 2, 11, 13, 19, or 21, wherein the polypeptide is soluble in the absence of denaturing agents. In some embodiments, the nucleic acid encodes a polypeptide comprising at least about 95% identity to any one of SEQ ID NOS: 2, 11, 13, 19, or 21 or amino acids 42-743 of SEQ ID NO: 29 or amino acids 64-765 of SEQ ID NO: 32, or encodes the polypeptide of SEQ ID NOS: 2, 11, 13, 19, or 21 or amino acids 42-743 of SEQ ID NO: 29 or amino acids 64-765 of SEQ ID NO: 32. In some aspects, the nucleic acid comprises one of SEQ ID NOS: 1, 10, 12, 18, or 20.

In some aspects, an isolated polynucleotide of the invention comprises a codon optimized coding region encoding any one of SEQ ID NOS: 2, 11, 13, 19, or 21 or amino acids 42-743 of SEQ ID NO: 29 or amino acids 64-765 of SEQ ID NO: 32, or a fragment, variant, analog or derivative thereof, optimized for codon usage in the host in which the polynucleotide is expressed. For example, in some embodiments the coding region is codon-optimized for expression in E. coli. In some embodiments, the E. coli-optimized coding region encoding SEQ ID NO: 2 comprises a nucleic acid sequence wherein one or more codons are optimized, e.g., about 65-69 of the 72 alanine codons in the coding region are GCG and about 3-7 of the alanine codons are GCC; about 5-7 of the 7 cysteine codons in the coding region are TGC and about 0-2 of the cysteine codons are TGT; about 31-34 of the 34 aspartic acid codons in the coding region are GAT and about 0-3 of the aspartic acid codons are GAC; about 21-23 of the 23 glutamic acid codons in the coding region are GAA and about 0-2 of the glutamic acid codons are GAG; about 25-29 of the 33 phenylalanine codons in the coding region are TTC and about 4-8 of the phenylalanine codons are TTT; about 60-64 of the 87 glycine codons in the coding region are GGT and about 23-27 of the glycine codons are GGC; about 4-6 of the 6 histidine codons in the coding region are CAT and about 0-2 of the histidine codons are CAC; about 20-24 of the 37 isoleucine codons in the coding region are ATT and about 13-17 of the isoleucine codons are ATC; about 26-28 of the 28 lysine codons in the coding region are AAA and about 0-2 of the lysine codons are AAG; about 62-64 of the 64 leucine codons in the coding region are CTG; about 47-51 of the 61 asparagine codons in the coding region are AAC and about 10-14 of the asparagine codons are AAT; about 32-34 of the 34 proline codons in the coding region are GAT; about 27-30 of the 30 glutamine codons in the coding region are CAG and about 0-3 of the glutamine codons are CAA; about 9-13 of the 17 arginine codons in the coding region are CGT and about 4-8 of the arginine codons are CGC; about 43-47 of the 83 serine codons in the coding region are AGC and about 36-40 of the serine codons are TCT; about 50-54 of the 54 threonine codons in the coding region are ACC and about 0-4 of the threonine codons are ACG; about 24-28 of the 47 valine codons in the coding region are GTT and about 19-23 of the valine codons are GTG; and/or about 12-16 of the 26 tyrosine codons in the coding region are TAT and about 10-14 of the tyrosine codons are TAC.

In some embodiments, the E. coli-optimized coding region encoding SEQ ID NO: 2 comprises a nucleic acid sequence wherein one or more codons are codon-optimized, e.g.,: about 67 of the 72 alanine codons in the coding region are GCG and about 5 of the alanine codons are GCC; about 7 of the 7 cysteine codons in the coding region are TGC; about 33 of the 34 aspartic acid codons in the coding region are GAT and about 1 of the aspartic acid codons are GAC; about 23 of the 23 glutamic acid codons in the coding region are GAA; about 27 of the 33 phenylalanine codons in the coding region are TTC and about 6 of the phenylalanine codons are TTT; about 62 of the 87 glycine codons in the coding region are GGT and about 25 of the glycine codons are GGC; about 6 of the 6 histidine codons in the coding region are CAT; about 22 of the 37 isoleucine codons in the coding region are ATT and about 15 of the isoleucine codons are ATC; about 28 of the 28 lysine codons in the coding region are AAA; about 64 of the 64 leucine codons in the coding region are CTG; about 49 of the 61 asparagine codons in the coding region are AAC and about 12 of the asparagine codons are AAT; about 34 of the 34 proline codons in the coding region are GAT; about 29 of the 30 glutamine codons in the coding region are CAG and about 1 of the glutamine codons are CAA; about 11 of the 17 arginine codons in the coding region are CGT and about 6 of the arginine codons are CGC; about 45 of the 83 serine codons in the coding region are AGC and about 38 of the serine codons are TCT; about 52 of the 54 threonine codons in the coding region are ACC and about 2 of the threonine codons are ACG; about 26 of the 47 valine codons in the coding region are GTT and about 21 of the valine codons are GTG; and/or about 14 of the 26 tyrosine codons in the coding region are TAT and about 12 of the tyrosine codons are TAC. In one aspect, the isolated nucleic acid of the invention comprises SEQ ID NO:3.

The present invention is also directed to an isolated nucleic acid which hybridizes, upon incubation in a solution comprising 50% formamide at about 37° C., to a DNA sequence which is complementary to any one of SEQ ID NO: 1 or SEQ ID NO: 3, wherein the nucleic acid encodes a polypeptide which is soluble in the absence of denaturing agents, and wherein the polypeptide is recognized by an antibody that specifically binds to a polypeptide consisting of SEQ ID NO: 2. In some embodiments, the present invention is directed to an isolated nucleic acid which hybridizes, upon incubation in a solution comprising 50% formamide at about 37° C., to a DNA sequence which is complementary to SEQ ID NO: 10, wherein said nucleic acid encodes a polypeptide which is soluble in the absence of denaturing agents, and wherein said polypeptide is recognized by an antibody that specifically binds to a polypeptide consisting of SEQ ID NO: 11. In some embodiments, the present invention is directed to an isolated nucleic acid which hybridizes, upon incubation in a solution comprising 50% formamide at about 37° C., to a DNA sequence which is complementary to SEQ ID NO: 12, wherein said nucleic acid encodes a polypeptide which is soluble in the absence of denaturing agents, and wherein said polypeptide is recognized by an antibody that specifically binds to a polypeptide consisting of SEQ ID NO: 13. In some embodiments, the present invention is directed to an isolated nucleic acid which hybridizes, upon incubation in a solution comprising 50% formamide at about 37° C., to a DNA sequence which is complementary to SEQ ID NO: 18, wherein said nucleic acid encodes a polypeptide which is soluble in the absence of denaturing agents, and wherein said polypeptide is recognized by an antibody that specifically binds to a polypeptide consisting of SEQ ID NO: 19. In some embodiments, the present invention is directed to an isolated nucleic acid which hybridizes, upon incubation in a solution comprising 50% formamide at about 37° C., to a DNA sequence which is complementary to SEQ ID NO: 20, wherein said nucleic acid encodes a polypeptide which is soluble in the absence of denaturing agents, and wherein said polypeptide is recognized by an antibody that specifically binds to a polypeptide consisting of SEQ ID NO: 21.

In some embodiments, the nucleic acid is fused (including, but not limited to, ligated) to a heterologous nucleic acid. For example, in some embodiments the heterologous nucleic acid encodes a heterologous polypeptide which is fused to the polypeptide encoded by the nucleic acid. In some embodiments, the heterologous polypeptide which is fused to the polypeptide encoded by the nucleic acid is selected from the group consisting of His-tag, a ubiquitin tag, a NusA tag, a chitin binding domain, ompT, ompA, pelB, DsbA, DsbC, c-myc, KSI, polyaspartic acid, (Ala-Trp-Trp-Pro)n (SEQ ID NO:16), polyphenyalanine, polycysteine, polyarginine, a B-tag, a HSB-tag, green fluorescent protein (GFP), an influenza virus hemagglutinin (HAI), a calmodulin binding protein (CBP), a galactose-binding protein, a maltose binding protein (MBP), cellulose binding domains (CBD's), dihydrofolate reductase (DHFR), glutathione-5-transferase (GST), streptococcal protein G, staphylococcal protein A, T7gene10, an avidin/streptavidin/Strep-tag, trpE, chloramphenicol acetyltransferase, lacZ (β-Galactosidase), a His-patch thioredoxin, thioredoxin, a FLAG™ peptide (Sigma Aldrich), an S-tag, and a T7-tag, and a combination of two or more of said heterologous polypeptides. The nucleic acid of the present invention can be fused to a heterologous nucleic acid, for instance, to increase the stability and/or to facilitate the isolation and/or purification of the nucleic acid or the expressed polypeptide.

In some embodiments, the heterologous nucleic acid comprises a promoter operably associated with the nucleic acid of the invention. For instance, the present invention includes a nucleic acid under the control of a Salmonella promoter (e.g., ssaG promoter). In another embodiment, the nucleic acid of the invention is under the control of a viral promoter (e.g., Modified Vaccinia Ankara Virus promoter and Moloney Murine Leukemia Virus promoter) or eukaryotic promoter. In other embodiments, the nucleic acid of the invention further comprises a Chlamydial promoter from the same Chlamydial species and/or serotype as the nucleic acid.

The present invention is also directed to a vector comprising the nucleic acid of the present invention. In some embodiments, the vector further comprises a promoter operably associated with the nucleic acid. In some embodiments, the vector is a plasmid. For example, in some embodiments the plasmid is a pLex plasmid.

In some embodiments of the present invention, the polypeptide of the invention induces a protective immune response when administered to an animal. The immune response can be a cellular and/or humoral immune response.

The invention is also directed to a host cell comprising the vector of the present invention. In some embodiments, the host cell is selected from the group consisting of bacterial cells, mammalian cells, yeast cells, insect cells, or plant cells. In some embodiments, the bacterial cell selected from the group consisting of Escherichia coli, Bacillus subtilis, Salmonella typhimurium, Pseudomonas aeruginosa or Pseudomonas fluorescens.

In yet another embodiment, the invention includes a host cell comprising the nucleic acid, wherein the host cell is capable of expressing the Chlamydial polypeptide encoded by the nucleic acid. In one embodiment, the nucleic acid of the invention is integrated into the host genome. For instance, the nucleic acid can be cloned into a gene expression cassette which is integrated into the host genome by homologous recombination. In one embodiment, the nucleic acid is integrated into a viral genome, for instance, a Modified Vaccinia Ankara Virus or Moloney Murine Leukemia Virus genome. In another embodiment, the nucleic acid is integrated into a non-Chlamydial bacterial genome, for instance, a Salmonella enterica genome. In one embodiment, the host cell is modified so that it is avirulent when administered to an animal.

The present invention is also directed to a method of producing a polypeptide with at least about 70%, 75%, 80%, 85%, 90%, 95% or even 99% sequence identity to any one of SEQ ID NOS:2, 11, 13, 19, or 21, wherein the polypeptide is soluble in the absence of denaturing agents, comprising culturing the host cell of the present invention, and recovering the polypeptide.

In some embodiments, the invention is directed to a polypeptide encoded by the polynucleotide of the present invention. For instance, the present invention includes a polypeptide with at least about 70%, 75%, 80%, 85%, 90%, 95% or even 99% sequence identity to any of SEQ ID NOS: 2, 11, 13, 19 and 21 or amino acids 42-743 of SEQ ID NO: 29 or amino acids 64-765 of SEQ ID NO: 32. In one embodiment, the invention includes an isolated, soluble, truncated Chlamydial Pmp polypeptide that lacks a N-terminal signal sequence and a C-terminal transmembrane domain. For instance, the invention includes a soluble, C. trachomatis and C. pneumoniae PmpG, PmpD and PmpH polypeptide that lacks a N-terminal signal sequence and a C-terminal transmembrane domain. In one embodiment, the PmpG polypeptide is a C. pneumoniae PmpG polypeptide which comprises a polypeptide with at least about 70%, 75%, 80%, 85%, 90%, 95% or even 99% amino acid sequence identity to amino acids 42-743 of SEQ ID NO: 29 or amino acids 64-765 of SEQ ID NO: 32.

The invention is also directed to a composition comprising a polypeptide of the present invention and a carrier. In some embodiments, the composition of the present invention comprises a nucleic acid of the present invention or a vector of the present invention, and a carrier. In some embodiments, the composition of the present invention comprises a polypeptide of the present invention and a carrier. In other embodiments, the composition of the present invention comprises a host cell capable of expressing the polypeptide of the present invention and a carrier.

In some embodiments, the composition of the present invention is an immunogenic composition. In other embodiments, the composition of the present invention is a pharmaceutical composition. In yet other embodiments, the composition of the present invention is a vaccine.

In some embodiments, the composition further comprises an adjuvant. In some embodiments, the adjuvant is selected from the group consisting of: alum, bentonite, latex and acrylic particles, pluronic block polymers, squalene, depot formers, surface active materials, lysolecithin, retinal, Quil A, liposomes, and pluronic polymer formulations; macrophage stimulators, alternate pathway complement activators, non-ionic surfactants bacterial components, aluminum-based salts; calcium-based salts; silica; polynucleotides; toxoids; serum proteins, viruses and virally-derived materials, poisons, venoms, imidazoquiniline compounds, poloxamers, mLT, cationic lipids, and Qs21. In some embodiments, the adjuvant is a toll-like receptor (TLR) stimulating adjuvant. TLR adjuvants include compounds that stimulate the TLRs (e.g., TLR1-TLR13), resulting in an increased immune system response to the vaccine composition of the present invention. TLR adjuvants include, but are not limited to CpG (Coley Pharmaceutical Group Inc.) and MPL (Corixa).

The present invention is also directed to a kit comprising the polypeptide of the present invention and a means for administering the polypeptide. In one embodiment, the invention includes a kit comprising an attenuated host cell transformed with the nucleic acid of the invention and a means for administering the attenuated host cell to an animal.

Some embodiments of the present invention are directed to a method of treating or preventing a Chlamydia infection in an animal comprising administering to the animal in need thereof a composition of the present invention. In some embodiments, the invention is directed to a method of treating or preventing a Chlamydia infection in an animal comprising administering to the animal in need thereof the polypeptide of the present invention. In some embodiments, the invention is directed to a method of treating or preventing a Chlamydia infection in an animal comprising administering to the animal in need thereof a nucleic acid of the present invention or a vector of the present invention. In some embodiments, the invention is directed to a method of treating or preventing a Chlamydia infection in an animal comprising administering to the animal in need thereof the attenuated host cell transformed with a Chlamydial nucleic acid of the invention.

In some embodiments, the invention is directed to a method of inducing an immune response against Chlamydia in an animal comprising administering an effective amount of a polypeptide of the invention, nucleic acid of the invention, vector of the invention, host cell of the invention, or composition of the invention. In some embodiments, the immune response is an antibody response. In some embodiments, the immune response is a T-cell response. In some embodiments, the immune response is a mucosal immune response. In some embodiments, the host animal is a human. In some embodiments, the administering is performed via mucosal delivery, transdermal delivery, subcutaneous injection, intravenous injection, oral administration, pulmonary administration, or via intradural injection.

The present invention is also directed to a method of producing a vaccine against Chlamydia comprising: (a) isolating the polypeptide of the present invention; and (b) adding an adjuvant to the isolated polypeptide of (a).

The present invention is also directed to an antibody specifically reactive with a Chlamydia organism, isolated from the serum of a host animal administered the polypeptide or polynucleotide of the present invention. In some embodiments, the invention is directed to a method of providing passive immunity comprising administering the antibody reactive with the Chlamydia organism to an animal in need thereof.

### BRIEF DESCRIPTION OF THE DRAWINGS/FIGURES

FIG. 1 shows the nucleic acid sequence (SEQ ID NO:1) and amino acid translation (SEQ ID NO:2) of CT84.

FIG. 2 is a plasmid map of pLEX-CT84. The CT84 gene fragment is inserted in the Nde I and Xba I restriction enzyme sites of the pLEX vector.

FIG. 3 shows the nucleic acid sequence (SEQ ID NO:3) of an E. coli-codon optimized sequence encoding SEQ ID NO:2.

FIG. 4A shows the nucleic acid sequence (SEQ ID NO:6) and amino acid translation (SEQ ID NO:7) of CT40, and FIG. 4B shows the nucleic acid sequence (SEQ ID NO:8) and amino acid translation (SEQ ID NO:9) of CT57.

FIG. 5 shows the nucleic acid sequence (SEQ ID NO:10) and the amino acid translation (SEQ ID NO:11) of PmpD-133.

FIG. 6 shows the nucleic acid sequence (SEQ ID NO:12) and the amino acid translation (SEQ ID NO:13) of PmpH-78.

FIG. 7 shows the nucleic acid sequence (SEQ ID NO:14) and the amino acid translation (SEQ ID NO:15) of PmpI-63.

FIG. 8 shows the nucleic acid sequence (SEQ ID NO:18) and the amino acid translation (SEQ ID NO:19) of OmcB-1.

FIG. 9 shows the nucleic acid sequence (SEQ ID NO:20) and the amino acid translation (SEQ ID NO:21) of OmpH-1.

FIG. 10 shows SDS-PAGE and Western blot analysis of CT84 expressed from pET15b-CT84.

FIG. 11 represents nickel affinity column purification of CT84, with Coomasie staining (bottom left) and Western blot analysis (bottom right).

FIG. 12A represents Superdex 200 gel filtration column purification of CT84 verified with Coomasie staining. FIG. 12B represents Superdex 200 gel filtration column purification of CT40 analyzed with Coomasie staining.

FIG. 13 represents Western blot analysis of purified CT40, CT57 and CT84 proteins.

FIG. 14A is an immune response graph indicating the IgG titer of CT110, CT84, CT57 and CT40. FIG. 14B shows the Chlamydia recovery following lung infection with CT110, CT84, CT57 and CT40.

FIG. 15 represents Western Blot analysis of the expression and purification of OmcB-1 and OmpH-1.

FIG. 16 represents Western Blot analysis of the expression and purification of PmpD-133, in BL21 cells and BL21(pLysS) cells.

FIG. 17 represents Western Blot analysis of theeluates of PmpI-63 using nickel speharose beads.

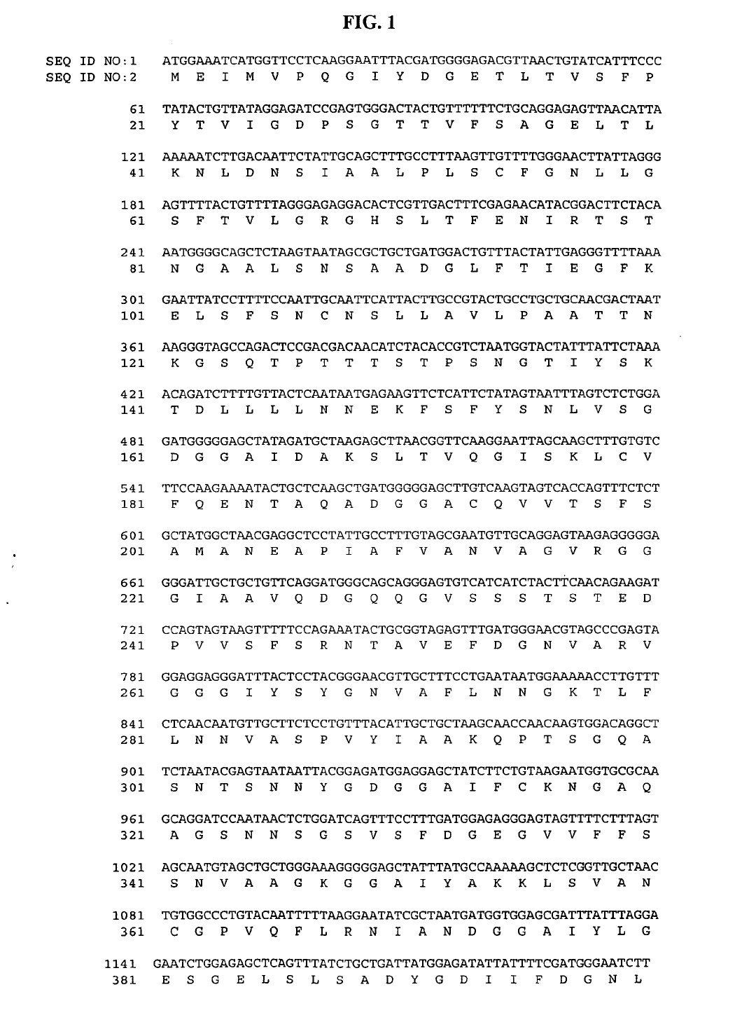

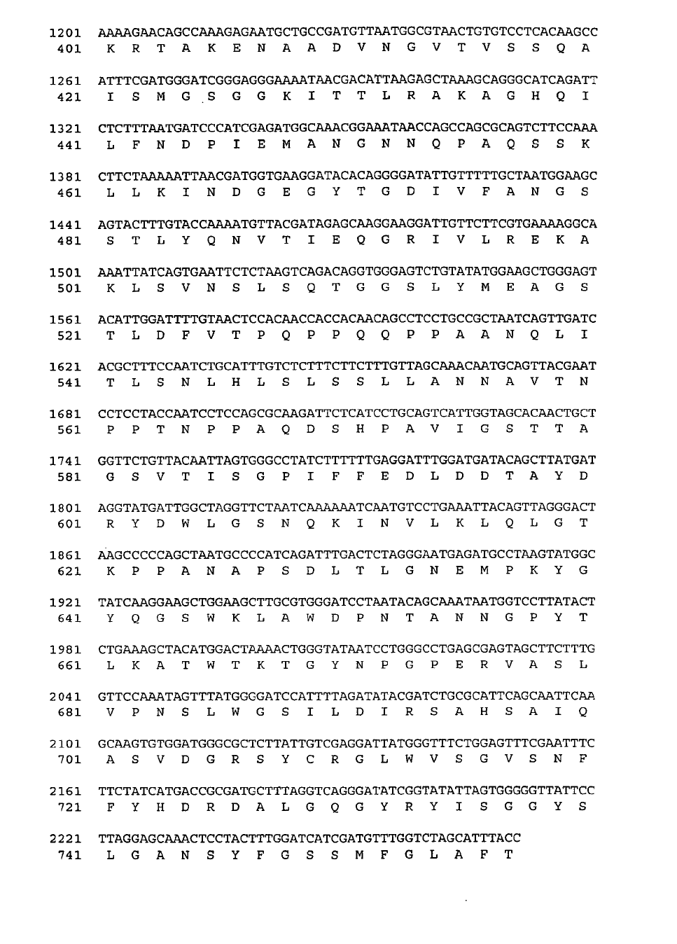

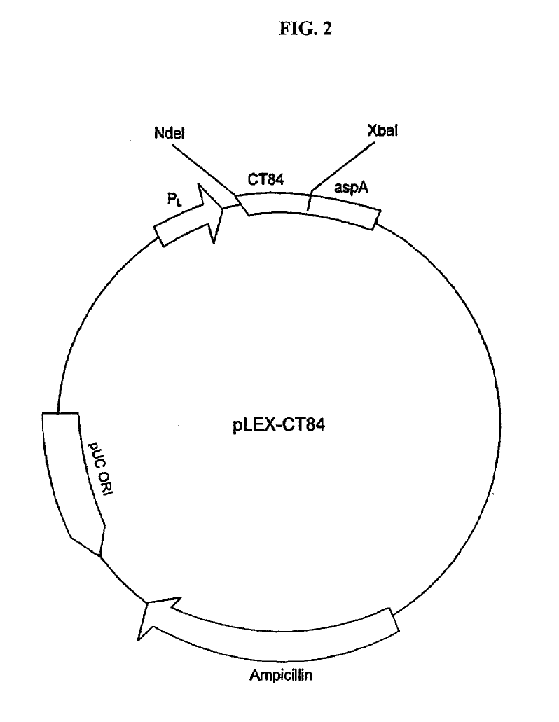

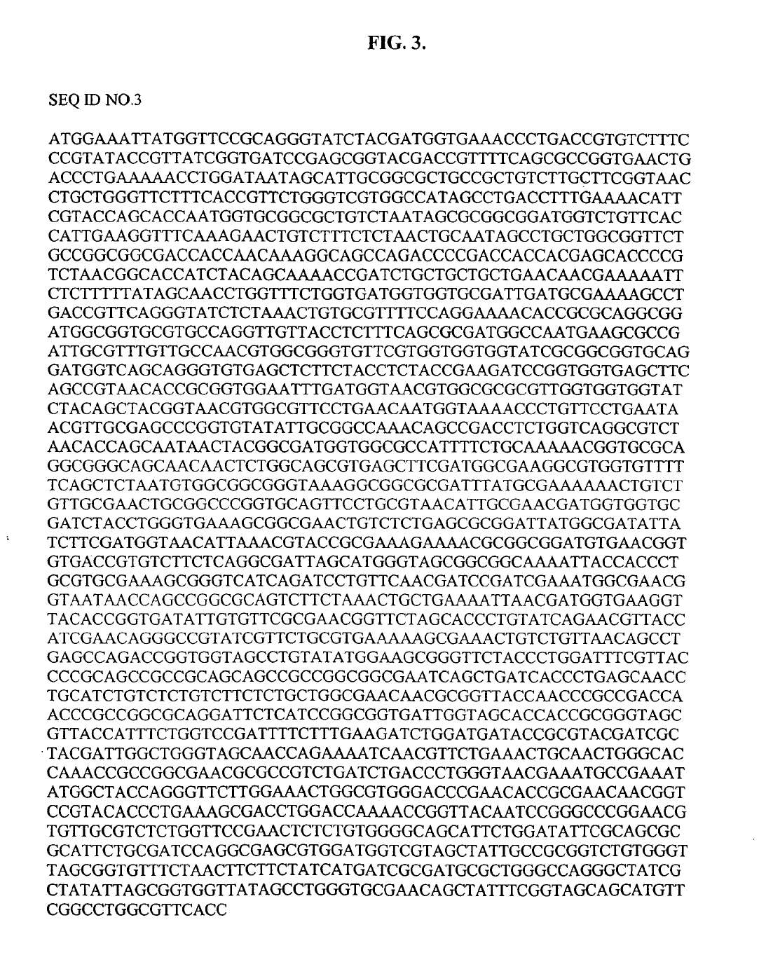

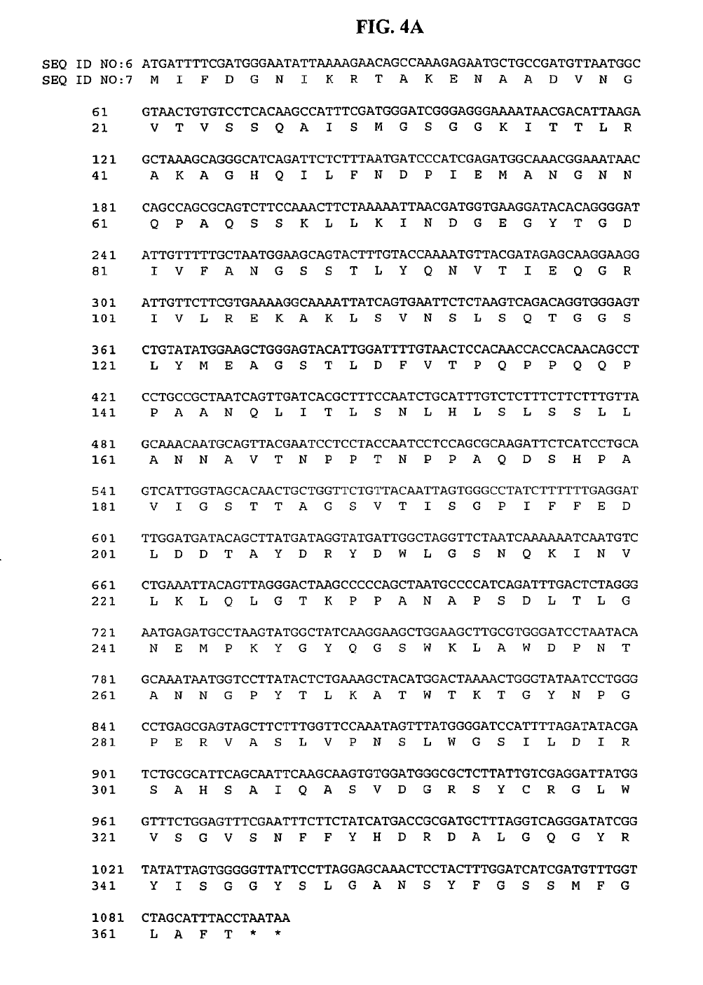

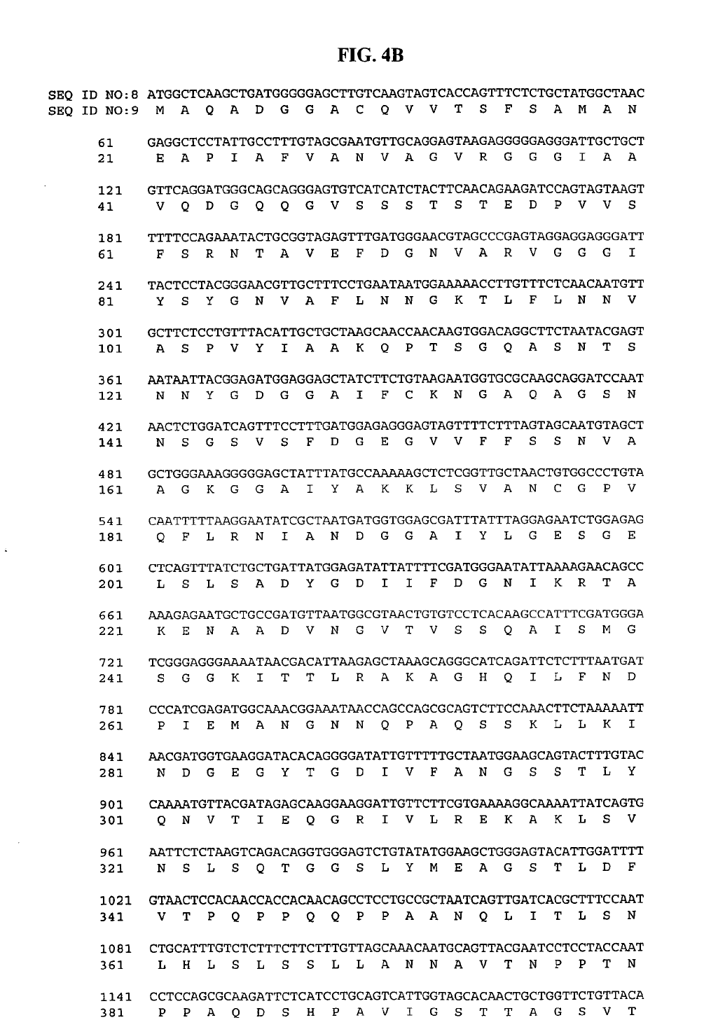

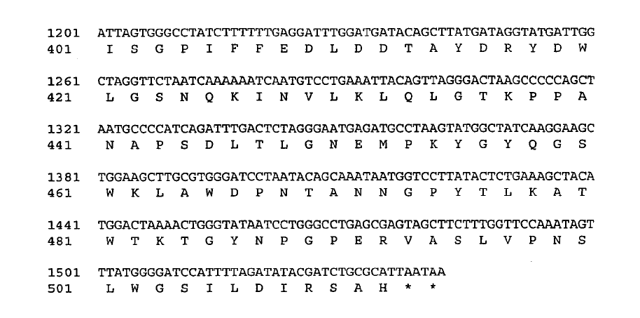

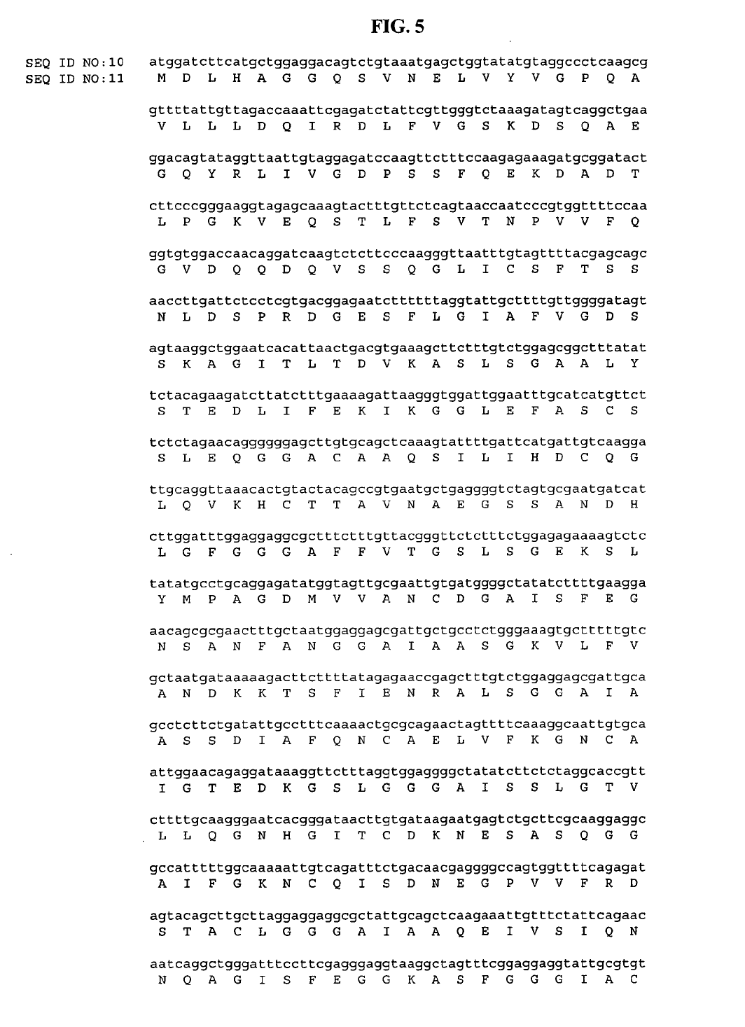

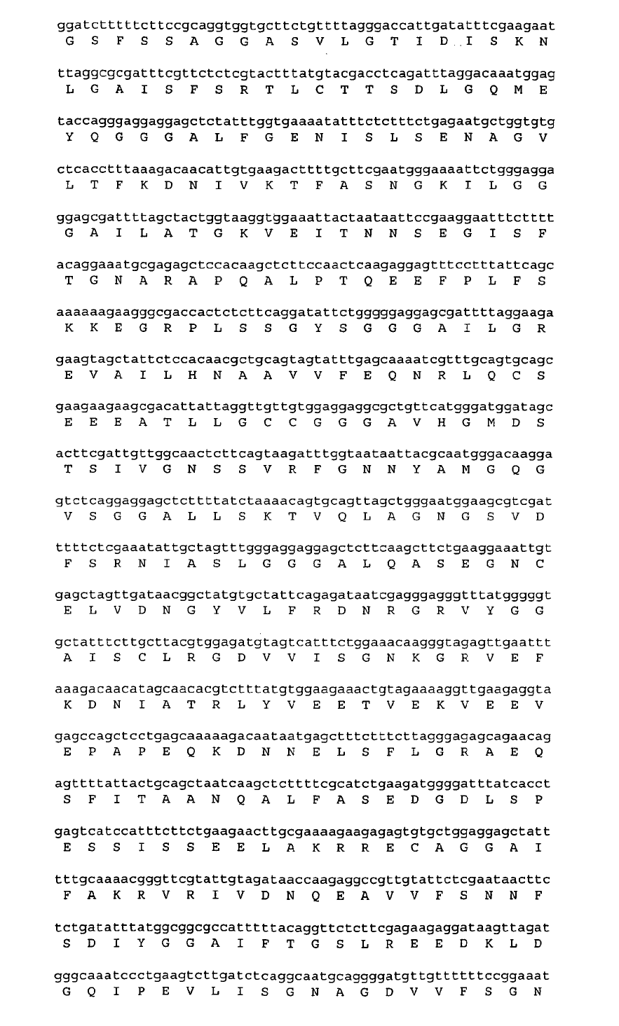

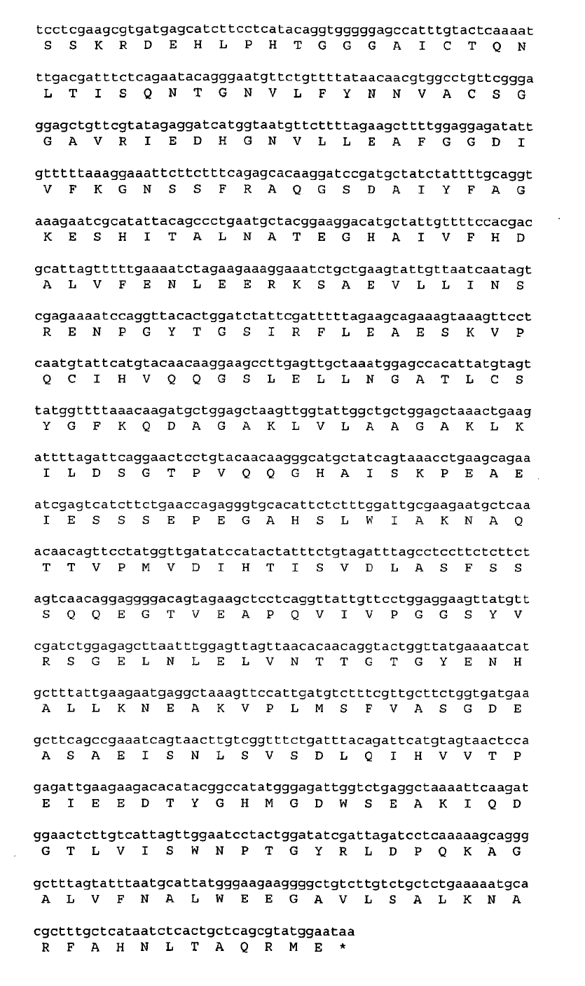

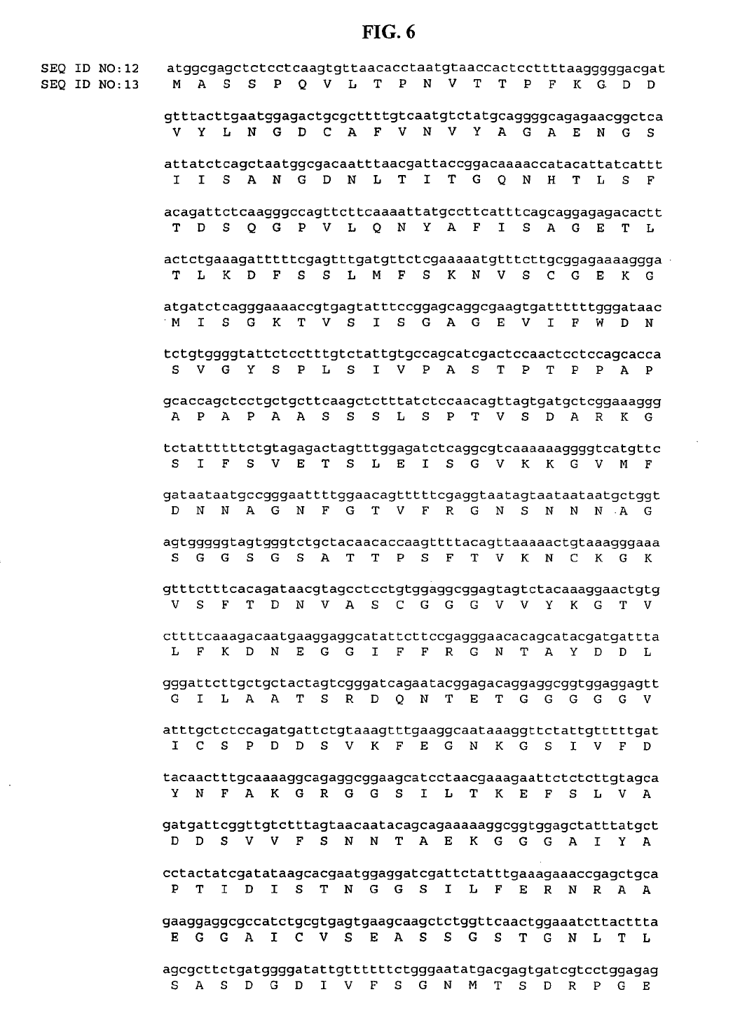

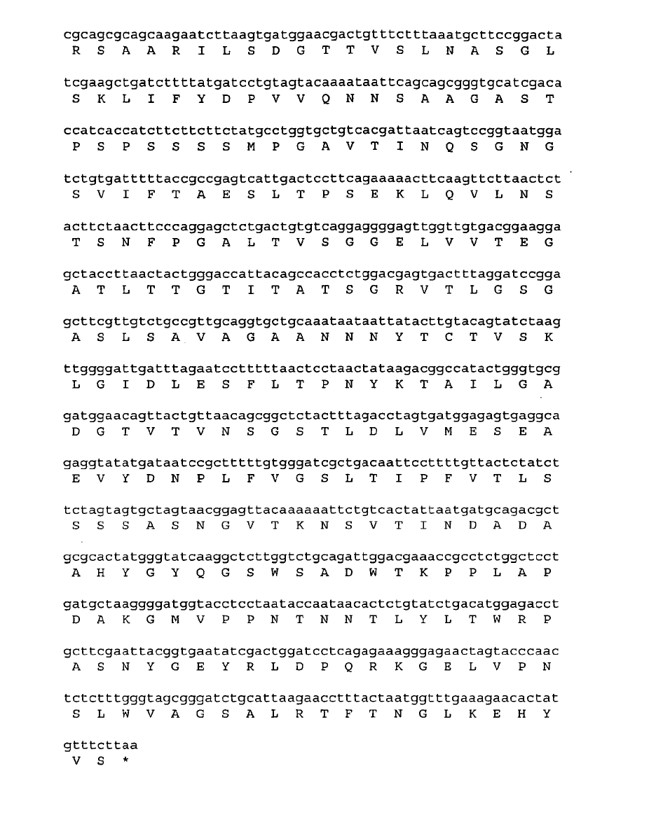

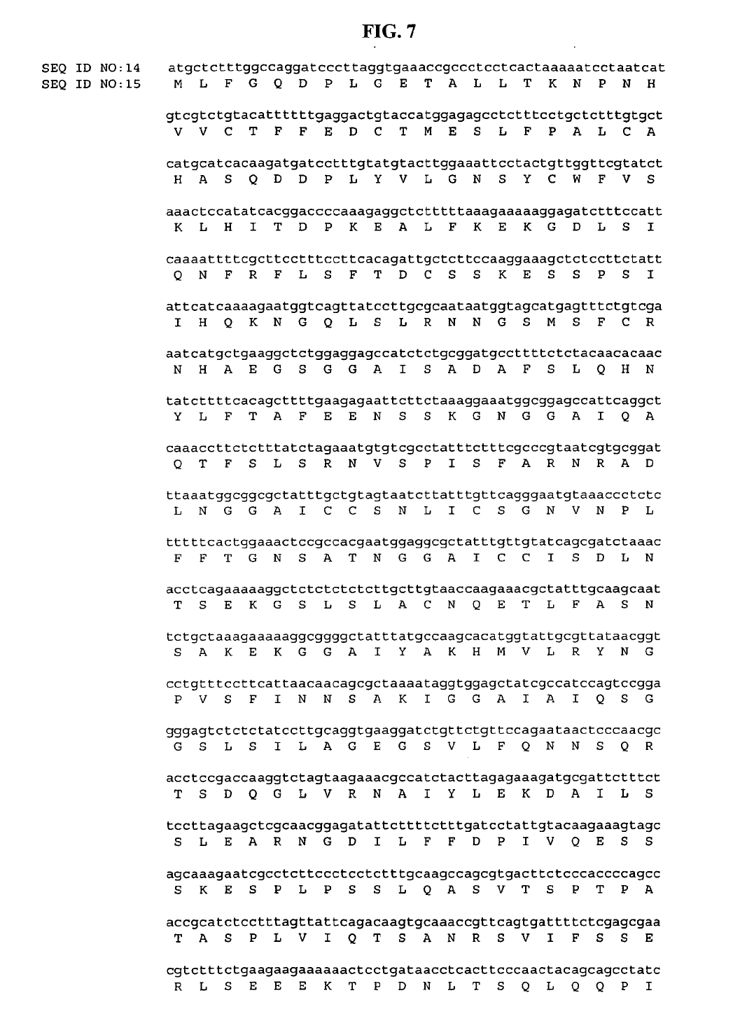

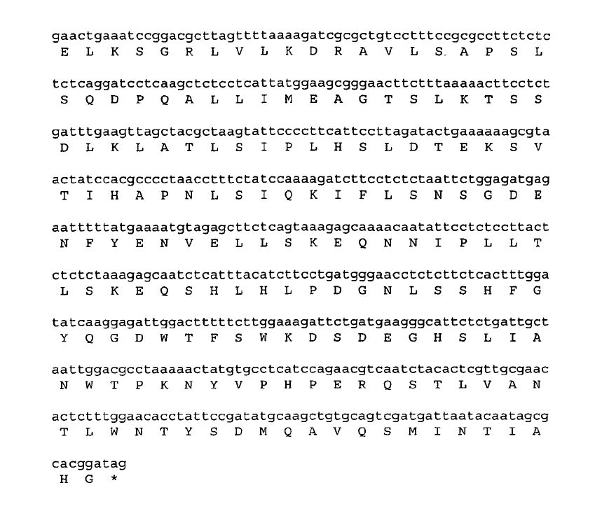

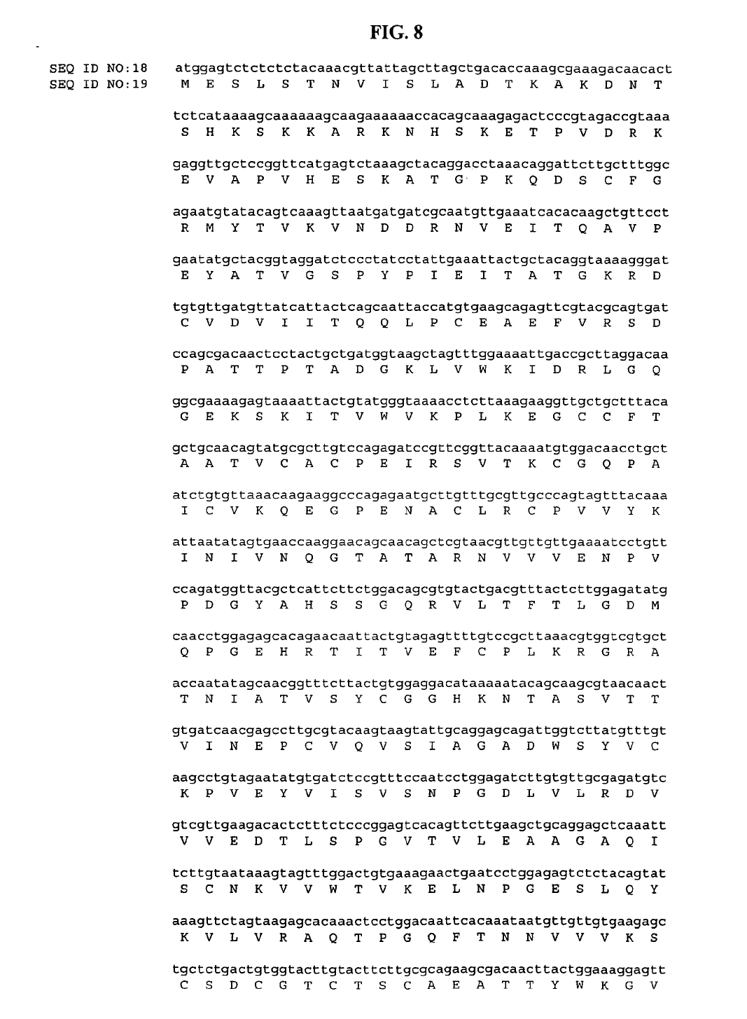

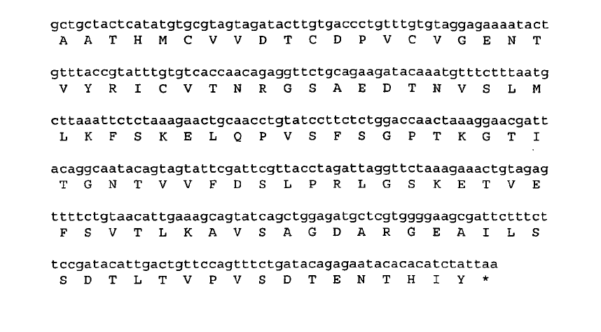

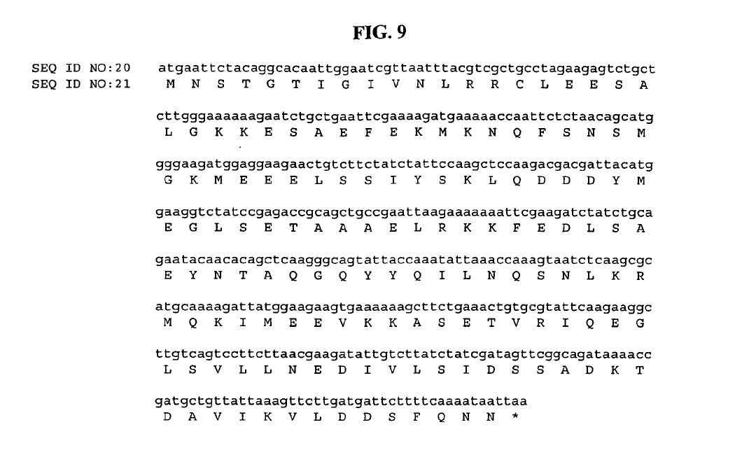

### DETAILED DESCRIPTION OF THE INVENTION

The present invention is directed to polypeptides and nucleic acids derived from the genus Chlamydia. Examples of suitable Chlamydia species include, but are not limited to, Chlamydia trachomatis, Chlamydia psittaci, Chlamydia percorum, Chlamydia muridarum, Chlamydia caviae, Chlamydia felis, Chlamydia abortus and Chlamydia pneumoniae.

Antigen candidates for a Chlamydia vaccine include CT110, PmpD, PmpH, PmpI, OmcB and OmpH. See e.g., Crane et al., Proc Natl Acad Sci 103:1894-9 (2006); Carlson et al., Infect Immun. 73:6407-18 (2005); Rocha et al., Nucleic Acids Res. 30:4351-60 (2002); Saren et al., Infect Immun. 70:3336-43 (2002); Christiansen et al., J Infect Dis. 181 Suppl 3:S528-37 (2000); and Westbay et al., Infect Immun. 62:5614-23 (1994).

CT110, also referred to as PmpG or HMW, is a mature 110 kDa membrane protein located in the outer membrane of Chlamydia trachomatis. See e.g., U.S. Pat. Nos. 6,887,843 and 6,642,023. However, this protein is not readily soluble in the absence of denaturing agents, thus making expression and purification more involved and expensive. Thus, it is an object of the present invention to provide polypeptides having the immunogenicity of CT110, but which remain soluble in the absence of denaturing agents.

One embodiment of the present invention is a genetically engineered truncated version of CT110 termed CT84, and fragments, variants, analogs, and derivatives thereof. CT84 is an 84 kDa fragment of CT110 that lacks the N-terminal signal peptide and the hydrophobic C-terminal membrane domain of CT110. Specifically, C. trachomatis CT84 (SEQ ID NO: 2) contains amino acids 29-784 of CT110, except that one additional amino acid (methionine) was added at the beginning of CT84 to initiate protein translation. The present invention further provides an isolated nucleic acid encoding a polypeptide comprising an amino acid with at least about 70%, 75%, 80%, 85%, 90%, 95%, 96%, 97%, 98%, or 99% sequence identity to SEQ ID NO: 2, wherein the polypeptide is soluble in the absence of denaturing agents.

Other variants of CT110 have been made, but have not been found to be soluble in the absence of denaturing agents. For example, CT40 (SEQ ID NO: 7), which comprises amino acid 422 to 784 of CT110, and CT57 (SEQ ID NO: 9), which comprises amino acid 213 to 724 of CT110, were not soluble in the absence of denaturing agents.

The invention includes C. pneumoniae CT84 homologs (amino acids 42-743 of SEQ ID NO: 29 and amino acids 64-765 of SEQ ID NO: 32). SEQ ID NOS: 30, 31 and 33 are reference nucleic acid sequences for C. pneumoniae J138, CWL029 and AR39 PmpG sequences, respectively. SEQ ID NO: 29 is a reference polypeptide sequence for C. pneumoniae J138 and CWL029 PmpG. SEQ ID NO: 32 is a reference polypeptide sequence for C. pneumoniae AR39PmpG.

PmpD, PmpH, and PmpI are 1531, 991 and 878 amino acid outer membrane proteins. Like CT110, these proteins are also not readily soluble in the absence of denaturing agents. Thus, it is an object of the present invention to provide polypeptides having the immunogenicity (or increased immunogenicity) of PmpD, PmpH, and/or PmpI, but which remain soluble in the absence of denaturing agents.

A genetically engineered truncated version of C. trachomatis PmpD was created, termed PmpD-133. PmpD-133 is a 126 kDa fragment of PmpD that lacks the N-terminal signal peptide and the hydrophobic C-terminal transmembrane domain of PmpD. Specifically, PmpD-133 (SEQ ID NO: 11) contains amino acids 33-1244 of PmpD, except that one additional amino acid (methionine) was added at the beginning of PmpD to initiate protein translation. The present invention further provides an isolated nucleic acid encoding a polypeptide with at least about 70%, 75%, 80%, 85%, 90%, 95%, 96%, 97%, 98%, or 99% sequence identity to SEQ ID NO: 11, wherein the polypeptide is soluble in the absence of denaturing agents.

A genetically engineered truncated version of C. trachomatis PmpH was also created, termed PmpH-78. PmpH-78 is a 71 kDa fragment of PmpH that lacks the N-terminal signal peptide and the hydrophobic C-terminal transmembrane domain of PmpH. Specifically, PmpH-78 (SEQ ID NO: 13) contains amino acids 24-724 of PmpH, except that one additional amino acid (methionine) was added at the beginning of PmpH to initiate protein translation. The present invention further provides an isolated nucleic acid encoding a polypeptide with at least about 70%, 75%, 80%, 85%, 90%, 95%, 96%, 97%, 98%, or 99% sequence identity to SEQ ID NO: 13, wherein the polypeptide is soluble in the absence of denaturing agents.

A genetically engineered truncated version of C. trachomatis PmpI was also created, termed PmpI-63. PmpI-63 is a 63 kDa fragment of PmpI that lacks the N-terminal signal peptide and the hydrophobic C-terminal transmembrane domain of PmpI. Specifically, PmpI-63 (SEQ ID NO: 15) contains amino acids 21-602 of PmpI, except that one additional amino acid (methionine) was added at the beginning of PmpI to initiate protein translation. The present invention further provides an isolated nucleic acid encoding a polypeptide with at least about 70%, 75%, 80%, 85%, 90%, 95%, 96%, 97%, 98%, or 99% sequence identity to SEQ ID NO: 15.

MOMP and OmcB are both members of the Chlamydial outer membrane complex. MOMP, which is an acronym for major outer membrane protein, is a 390 amino acid protein with an approximate molecular weight of 40 kDa. OmcB is a 60 kDa cysteine rich outer membrane protein containing 550 amino acids. A genetically engineered truncated version of C. trachomatis OmcB was created, termed OmcB-1 (SEQ ID NO: 19). OmcB-1 is a fragment of OmcB that lacks the N-terminal 36 amino acids, thus providing a OmcB protein without a signal sequence. The present invention further provides an isolated nucleic acid encoding a polypeptide with at least about 70%, 75%, 80%, 85%, 90%, 95%, 96%, 97%, 98%, or 99% sequence identity to SEQ ID NO:19, wherein the polypeptide is soluble in the absence of denaturing agents.

OmpH is a 19 KDa protein also found on the outer membrane. A genetically engineered truncated version of OmpH was created, termed OmpH-1 (SEQ ID NO: 21). OmpH-1 is a fragment of C. trachomatis OmpH that lacks the N-terminal 30 amino acids, thus providing an OmpH protein without a signal sequence. The present invention further provides an isolated nucleic acid encoding a polypeptide with at least about 70%, 75%, 80%, 85%, 90%, 95%, 96%, 97%, 98%, or 99% sequence identity to SEQ ID NO:21, wherein the polypeptide is soluble in the absence of denaturing agents.

### DEFINITIONS

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

As used herein, the terms “nucleic acid” or “polynucleotide” refer to deoxyribonucleotides or ribonucleotides. Unless specifically limited, the terms encompass nucleic acids containing analogues of natural nucleotides that have similar binding properties as the reference nucleic acid. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid encompasses polynucleotide, gene, cDNA, messenger RNA (mRNA), plasmid DNA (pDNA), or derivatives of pDNA (e.g., minicircles as described in (Darquet, A-M et al., Gene Therapy 4:1341-1349 (1997)). A nucleic acid may be provided in linear (e.g., mRNA), circular (e.g., plasmid), or branched form as well as double-stranded or single-stranded forms. A nucleic acid may comprise a conventional phosphodiester bond or a non-conventional bond (e.g., an amide bond, such as found in peptide nucleic acids (PNA)). The terms nucleic acid, polynucleotide, DNA and gene are used interchangeably herein.

“Codon optimization” is defined herein as modifying a nucleic acid sequence for enhanced expression in a specified host cell by replacing at least one, more than one, or a significant number, of codons of the native sequence with codons that are more frequently or most frequently used in the genes of that host.

As used herein, a “heterologous polynucleotide” or a “heterologous nucleic acid” or a “heterologous gene” or a “heterologous sequence” or an “exogenous DNA segment” refers to a polynucleotide, nucleic acid or DNA segment that originates from a source foreign to the particular host cell, or, if from the same source, is modified from its original form. A heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found.

As used herein, the term “isolated” means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the invention.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the invention. That is, e.g., a purified polypeptide of the present invention is a polypeptide that is at least about 70-100% pure, i.e., the polypeptide is present in a composition wherein the polypeptide constitutes about 70-100% by weight of the total composition. In some embodiments, the purified polypeptide of the present invention is about 75%-99% by weight pure, about 80%-99% by weight pure, about 90-99% by weight pure, or about 95% to 99% by weight pure.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example, promoters, ribosome binding sites, transcriptional terminators, and the like, are outside the coding region.

As used herein, the term “vector” refers to a nucleic acid molecule capable of transporting another nucleic acid to which it has been linked. Examples of vectors include, e.g., plasmids, viral vectors, cosmids and phagemids. Certain vectors are capable of autonomous replication in a host cell into which they are introduced. Other vectors are integrated into the genome of a host cell upon introduction into the host cell, and thereby are replicated along with the host genome.

As used herein, the term “plasmid” refers to a circular, double-stranded construct made up of genetic material (i.e., nucleic acids), wherein the genetic material is extrachromosomal and replicates autonomously.

The term “expression vector” refers to a vector that is capable of expressing the polypeptide of the present invention, i.e., the vector sequence contains the regulatory sequences required for polypeptide expression such as promoters, ribosome binding sites, etc. Expression vector and gene expression cassette are used interchangeably herein.

The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

The terms “fragment,” “variant,” “derivative” and “analog” when referring to Chlamydia polypeptides of the present invention include any polypeptides which retain at least some of the immunogenicity or antigenicity of the reference polypeptide (e.g., CT84 or CT110). Fragments of Chlamydia polypeptides of the present invention include proteolytic fragments, deletion fragments and in particular, fragments of Chlamydia polypeptides which exhibit increased solubility during expression, purification, and or administration to an animal. Polypeptide fragments further include any portion of the polypeptide which comprises an antigenic or immunogenic epitope of the native polypeptide, including linear as well as three-dimensional epitopes. Variants of Chlamydia polypeptides of the present invention include fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Variant polypeptides may comprise conservative or non-conservative amino acid substitutions, deletions or additions. Derivatives of Chlamydia polypeptides of the present invention are polypeptides which have been altered so as to exhibit additional features not found on the native polypeptide. Examples include fusion proteins. An analog is another form of a Chlamydia polypeptide of the present invention. An example is a proprotein which can be activated by cleavage of the proprotein to produce an active mature polypeptide.

The term “soluble in the absence of denaturing agents” refers to the propensity of a polypeptide to be soluble in an aqueous-based environment when denaturing agents are not present. Solubility of the protein can occur to varying degrees. Thus, in the present invention the term “soluble in the absence of denaturing agents” includes polypeptides that are greater than about 60%, 65%, 70%, 75%, 80%, 85%, 90%, 91%, 92%, 93%, 94%, 95%, 96%, 97%, 98%, 99%, 99.5% or 99.9% solubilized in an aqueous based solvent devoid of denaturing agents. Methods for determining solubility are known to those in the art, and can include fluorescence, spectroscopy, precipitation (e.g., centrifugation) assays, filtration assays, and degradation assays. In some embodiments, the solubility of a protein can be achieved by visual inspection for identification of proteins precipitating in a solution, indicating they are insoluble. While not being bound by a particular theory, denaturing agents unfold (either partially or entirely) a polypeptide from its native teritary conformation, resulting in decreased solubility of the polypeptide. In some embodiments the term “soluble in the absence of a denaturing agent” can refer to a propensity of a polypeptide to remain in its native tertiary structure, or to renature to its native tertiary structure, in the absence of a denaturing agent. Thus, in some embodiments, the polypeptide of the present invention can be denatured and purified using a denaturing agent, but upon removal of the denaturing agent the polypeptide would renature and be soluble.

The term “absence of denaturing agents” refers to an environment in which a denaturing agent such as a chaotropic agent and/or detergent is substantially not present. The term “denaturing agents” refers to compounds or compositions which denature, or destroy the tertiary structure, of polypeptides or proteins. Examples of denaturing agents include, but are not limited to, chaotropic agents, detergents, and high salt concentrations. A chaotropic agent is an agent which causes molecular structure to be disrupted; in particular, those structures formed by nonbonding forces such as hydrogen bonding, Van der Waals interactions, and hydrophobic effects. Examples of chaotropic agents include, but are not limited to, urea, guanidine HCl, and high salt concentrations (e.g., >2M), such as salts containing, e.g., SCN⁻, H₂PO₄⁻, HSO₄⁻, HCO₃⁻, I⁻, Cl⁻, NO₃⁻, NH₄⁺, Cs⁺, K⁺, and (CH₃)₄N⁺ (tetramethylammonium) ions. In some embodiments, the term “absence of denaturing agents” refers to an environment substantially free of a chaotropic agent, e.g., urea or guanidine hydrochloride, i.e., an environment comprising <0.5M, <0.1M, <50 mM, <10 mM, <1 mM, <100 μM, <10 μM, or <1 μM of urea or guanidine hydrochloride.

The term detergent refers to amphipathic molecule having a nonpolar “tail” having aliphatic or aromatic character and a polar “head”. Detergents used in purification of proteins and polypeptides are known to those in the art and include, but are not limited to, nonionic detergents, e.g., NP40, Triton X-100, Triton X-114, Brij®-35 (Pierce Chemical, Rockford, Ill.), Brij®-58 (Pierce Chemical), Tween-20, Tween-80, octylglucoside, octylthioglucoside, Octaethylene glycol, decathylene glycol monododecyl ether, N-decanoyl-N-methylglucamine, polyoxyethylene based detergents, Span 20 (Sigma Aldrich, St. Louis, Mo.), Span 40 (Sigma Aldrich), Span 60 (Sigma Aldrich), Span 65 (Sigma Aldrich), Span 85 (Sigma Aldrich), tergitol, tetradecyl-(3-D-maltoside, and triethylene glycol; anionic detergents, e.g., sodium dodecylsulfate (SDS), deoxycholate, cholic acid, dehydrocholic acid, N,N-dimethyldodecylamine N-oxide, docusate sodium salt, glycocholic acid, N-laurylsarcosine, Niaproof 4, Triton QS-15, Triton QS-44, 1-octanesulfonic acid, sodium deoxycholate; cationic detergents, e.g., alkyltrimethylammonium bromide, benzalkonium chloride, benzyldimethylhexadecylammonoim chloride, dodecylethyldimethylammonium bromide, Girard's reagent, N,N′,N′-polyoxyethylene(10)-N-tallow-1,3-diaminopropane, thonzonium bromide, and trimethyl(tetradecyl)ammonium bromide; and zwitterionic detergents, e.g., CHAPS, CHAPSO, 3-(decyldimethylammonio)propanesulfonate, or #-(N,N-dimethylmyristylammonio)propanesulfonate. In some embodiments, the term “absence of denaturing agents” refers to an environment substantially free of a detergent, i.e., an environment comprising <0.01%, <0.005%, <0.001%, <0.0005%, <0.0001%, <0.00005%, or <0.00001% of detergent.

The term “soluble when expressed in E. coli” refers to the propensity of a polypeptide to substantially localize to the hydrophilic or aqueous-based environments of the gram-negative host, e.g., the cytoplasm, periplasm or extracellular medium. Thus, during cellular fractionation, a polypeptide “soluble when expressed in E. coli” would generally be substantially isolated with the cytoplasmic, periplasmic, or extracellular components of a host cell. One of skill in the art will recognize that neither the cellular localization of a polypeptide, nor the cellular fractionation of a polypeptide, is absolute. Thus, the phrase “substantially localize” refers to a polypeptide in which about 70%, 75%, 80%, 85%, 90%, 95%, 96%, 97%, 98% or 99% of the polypeptide is in the designated cellular location, e.g., cytoplasm, periplasm, or extracellular medium. One of skill in the art will recognize that in some embodiments, the solubility of a protein can vary according to what type of expression system is used. In some embodiments, the present invention is directed to a polypeptide comprising at least about 70%, 75%, 80%, 85%, 90%, 95%, 96%, 97%, 98%, or 99% sequence identity to any one of SEQ ID NO: 2, 11, 13, 19 or 21 or amino acids 42-743 of SEQ ID NO: 29 or amino acids 64-765 of SEQ ID NO: 32, wherein said polypeptide is soluble when expressed in any one of the expression systems identified herein. For example, in some embodiments, the present invention is directed to a polypeptide comprising at least about 70%, 75%, 80%, 85%, 90%, 95%, 96%, 97%, 98%, or 99% sequence identity to any one of SEQ ID NO: 2, 11, 13, 19 or 21 or amino acids 42-743 of SEQ ID NO: 29 or amino acids 64-765 of SEQ ID NO: 32, wherein said polypeptide is soluble when expressed in a prokaryote (e.g., Bacillus subtilis; Salmonella enterica, e.g., Salmonella typhimurium or Salmonella typhi; E. coli; Pseudomonas spp., e.g., P. aeruginosa or P. fluorescens (e.g. PFēnex™ (Dowpharma)); Streptomyces spp.; or Staphylococcus spp. In some embodiments, the present invention is directed to a polypeptide comprising at least about 70%, 75%, 80%, 85%, 90%, 95%, 96%, 97%, 98%, or 99% sequence identity to any one of SEQ ID NO: 2, 11, 13, 19 or 21 or amino acids 42-743 of SEQ ID NO: 29 or amino acids 64-765 of SEQ ID NO: 32, wherein said polypeptide is soluble when expressed in P. fluorescens.

The term “epitope” is intended to encompass a single epitope or multiple epitopes, and refers to portions of a polypeptide having antigenic or immunogenic activity in an animal, for example a mammal, including, but not limited to, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind as determined by any method well known in the art. Immunospecific binding excludes non-specific binding but does not necessarily exclude cross-reactivity with other antigens. Whereas all immunogenic epitopes are antigenic, antigenic epitopes need not be immunogenic due to, for instance, size or conformation.

The term “antigen” is intended to encompass a single antigen or multiple antigens (and its related term “antigenic”) and as used herein refers to a substance that binds specifically to an antibody or T-cell receptor. In some embodiments an antigen is immunogenic.

As used herein, an “immune response” refers to the ability of an animal to mount an immune reaction to a composition delivered to the animal. Examples of immune responses include an antibody response or a cellular, e.g., T-cell, response. Immune responses can also include a mucosal response, e.g., a mucosal antibody response, e.g., 5-IgA production or a mucosal cell-mediated response, e.g., T-cell response. Immune responses can also be humoral.

The term “peptide vaccine” or “subunit vaccine” refers to a composition comprising one or more polypeptides of the present invention, which when administered to an animal are useful in stimulating an immune response against Chlamydia infection.

The term “DNA vaccine,” “nucleic acid vaccine” or “polynucleotide vaccine” refers to composition comprising one or more nucleic acids encoding polypeptides of the present invention, which when administered to an animal, e.g., as naked DNA or in a viral vector, express one or more polypeptides of the present invention in the cells of the animal, thereby stimulating an immune response against a Chlamydia infection.

A multivalent vaccine refers to any vaccine prepared from two or more microorganisms or viruses, or alternatively, to a vaccine prepared from two or more polypeptides. When a multivalent vaccine comprises two or more polypeptides, the polypeptide can be from the same organism or from different organisms (e.g., C. pneumoniae and C. trachomatis).

The term “immunogenic carrier” as used herein refers to a first polypeptide or fragment, variant, or derivative thereof which enhances the immunogenicity of a second polypeptide, e.g., an antigenic epitope, or fragment, variant, or derivative thereof.

The term “adjuvant” refers to any material having the ability to (1) alter or increase the immune response to a particular antigen or (2) increase or aid an effect of a pharmacological agent. As used herein, any compound which may increase the expression, antigenicity or immunogenicity of an immunogen of the invention is a potential adjuvant. In some embodiments, the term adjuvant refers to a TLR stimulating adjuvant, wherein the TLR adjuvant includes compounds that stimulate the TLR receptors (e.g., TLR1-TLR13), resulting in an increased immune system response to the vaccine composition of the present invention. TLR adjuvants include, but are not limited to, CpG and MPL.

“Pharmaceutical compositions” comprise compositions containing nucleic acids, polypeptides, host cells or antibodies of the invention which are administered to an individual already suffering from a Chlamydia infection or at risk for a Chlamydia infection (i.e., anyone who has not been previously vaccinated or exposed to the specified Chlamydia species). As such, administration of a pharmaceutical composition can be used to treat or prevent a Chlamydia infection or condition associated with a Chlamydia infection. For instance, the pharmaceutical compositions of the invention can be useful for treating or preventing a Chlamydia trachomatis or Chlamydia pneumoniae infection.

“Pharmaceutically acceptable” refers to compositions and components of compositions (e.g., carriers, excipients, and adjuvants) that are, within the scope of sound medical judgment, suitable for contact with the tissues of human beings and animals without excessive toxicity or other complications commensurate with a reasonable benefit/risk ratio. In some embodiments, the polypeptide, polynucleotides, compositions, and vaccines of the present invention are pharmaceutically acceptable.

The terms “priming” or “primary” and “boost” or “boosting” as used herein may refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously.

The term “animal” is intended to encompass a singular “animal” as well as plural “animals” and comprises mammals and birds, as well as fish, reptiles, and amphibians. The term “mammal” is intended to encompass a singular “mammal” and plural “mammals,” and includes, but is not limited to, humans; primates such as apes, monkeys (e.g., owl, squirrel, cebus, rhesus, African green, patas, cynomolgus, and cercopithecus), orangutans, baboons, gibbons, and chimpanzees; canids such as dogs and wolves; felids such as cats, lions, and tigers; equids such as horses, donkeys, and zebras, food animals such as cows, pigs, and sheep; ungalates such as deer and giraffes; ursids such as bears; and rabbits, mice, ferrets, and whales. The term animal also encompasses model animals, e.g., disease model animals. In some embodiments, the term animal includes valuable animals, either economically or otherwise, e.g., economically important breeding stock, racing animals, show animals, heirloom animals, rare or endangered animals, or companion animals. In particular, the mammal can be a human subject, a food animal or a companion animal.

As used herein, an “animal in need thereof” or a “subject in need thereof” refers to an individual for whom it is desirable to treat, i.e., to prevent, cure, retard, or reduce the severity of Chlamydia disease symptoms, and/or result in no worsening of Chlamydia disease over a specified period of time.

The term “passive immunity” refers to the immunity to an antigen developed by a host animal, the host animal being given antibodies produced by another animal, rather than producing its own antibodies to the antigen. The term “active immunity” refers to the production of an antibody by a host animal as a result of the presence of the target antigen.

The term “sequence identity” as used herein refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window (e.g., SEQ ID NO: 2 and a homologous polypeptide from another C. trachomatis isolate). In order to optimally align sequences for comparison, the portion of a polynucleotide or polypeptide sequence in the comparison window may comprise additions or deletions termed gaps while the reference sequence (e.g. SEQ ID NO: 2) is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

### Polynucleotides

In some embodiments, the present invention is directed to a nucleic acid encoding a polypeptide comprising at least about 70%, 75%, 80%, 85%, 90%, 95%, 96%, 97%, 98%, or 99% identity to any one of SEQ ID NOS:2, 11, 13, 19, or 21 or amino acids 42-743 of SEQ ID NO: 29 or amino acids 64-765 of SEQ ID NO: 32, wherein the polypeptide is soluble in the absence of denaturing agents. As used herein, a polypeptide is “substantially homologous” if it comprises at least about 70%, 75%, 80%, 85%, 90%, 95%, 96%, 97%, 98%, or 99% identity to a reference sequence (e.g., SEQ ID NOS: 2, 11, 13, 19 or 21 or amino acids 42-743 of SEQ ID NO: 29 or amino acids 64-765 of SEQ ID NO: 32).

In certain embodiments, a nucleic acid of the present invention is DNA. In the case of DNA, a nucleic acid which encodes a polypeptide of the present invention can also comprise a promoter and/or other transcription or translation control elements operably associated with the nucleic acid. An operable association is when a nucleic acid encoding a gene product, e.g., a polypeptide, is associated with one or more regulatory sequences in such a way as to place expression of the gene product under the influence or control of the regulatory sequence(s). Two DNA fragments (such as a polypeptide-encoding nucleic acid and a promoter associated with the 5′ end of the nucleic acid) are “operably associated” if induction of promoter function results in the transcription of mRNA encoding the desired polypeptide and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example, enhancers, operators, repressors, and transcription termination signals, can be operably associated with the polynucleotide to direct cell-specific transcription. Suitable promoters and other transcription control regions are disclosed herein.

Certain polynucleotides of the present invention comprise a coding region which encodes a Chlamydia polypeptide described herein. Such coding regions can be isolated from their native source by PCR amplification and standard genetic manipulation techniques known by those in the art. For example, upon PCR amplification, the coding region can then be provided with appropriate linkers and ligated into expression vectors commonly available in the art, and the vectors may then be used to transform suitable hosts to produce a desired polypeptide of the present invention. A number of such vectors and suitable host systems are available. For expression of polypeptides of the present invention, the coding region will be provided with operably linked start and stop codons, promoter and terminator regions and usually a replication system to provide an expression vector for expression in the desired host. For example, for bacterial plasmids, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

Polynucleotides or nucleic acid sequences defined herein are represented by one-letter symbols for the bases as follows: A (adenine) C (cytosine) G (guanine) T (thymine) U (uracil) M (A or C)R (A or G) W (A or T/U); S(C or G); Y (C or T/U); K (G or T/U); V (A or C or G; not T/U); H (A or C or T/U; not G); D (A or G or T/U; not C); B (C or G or T/U; not A); N (A or C or G or T/U) or (unknown).

In some embodiments of the present invention the nucleic acid is isolated. For example, a recombinant nucleic acid contained in a vector is considered isolated for the purposes of the present invention. Further examples of an isolated nucleic acid include recombinant nucleic acids maintained in heterologous host cells or purified (partially or substantially) nucleic acids in solution. Isolated RNA molecules include in vivo or in vitro RNA transcripts of the nucleic acids of the present invention. Isolated nucleic acids according to the present invention further include such molecules produced synthetically.

### Codon Optimization

In some embodiments, the present invention is directed to an isolated nucleic acid which encodes one of the soluble CT84, PmpD-133, PmpH-78, OmcB-1, or OmpH-1 polypeptides from Chlamydia trachomatis, e.g., a polypeptide substantially homologous to SEQ ID NOS: 2, 11, 13, 19, or 21 respectively. In other embodiments, the present invention is directed to an isolated nucleic acid which encodes a Chlamydia pneumoniae polypeptide homologous to C. trachomatis CT-84, for instance, a polypeptide comprising at least about 70%, 75%, 80%, 85%, 90%, 95%, 96%, 97%, 98% or 99% sequence identity to amino acids 42-743 of SEQ ID NO: 29 or amino acids 64-765 of SEQ ID NO: 32.

As appreciated by one of ordinary skill in the art, various nucleic acid coding regions will encode the same polypeptide due to the redundancy of the genetic code. Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the polypeptides encoded by the DNA.

TABLE 1
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met (M) | ACG ” | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

It is to be appreciated that any polynucleotide that encodes a polypeptide in accordance with the invention falls within the scope of this invention, irregardless of the codons used.

Many organisms display a bias for use of particular codons to code for insertion of a particular amino acid in a growing polypeptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, the present invention is directed towards a polynucleotide wherein the coding region encoding the polypeptide of the present invention is codon-optimized.

The present invention relates to nucleic acids comprising codon-optimized coding regions which encode soluble Chlamydia trachomatis polypeptides with at least about 70%, 75%, 80%, 85%, 90%, 95%, 96%, 97%, 98%, or 99% sequence identity to any one of SEQ ID NOS: 2, 11, 13, 19, or 21 or amino acids 42-743 of SEQ ID NO: 29 or amino acids 64-765 of SEQ ID NO: 32, with the codon usage adapted for optimized expression in the cells of a given prokaryote or eukaryote. These polynucleotides are prepared by incorporating codons preferred for use in the genes of a given species into the DNA sequence. Also provided are polynucleotide expression constructs, vectors, gene expression cassettes, host cells comprising nucleic acids of codon-optimized coding regions which encode Chlamydia trachomatis polypeptides, and various methods of using the polynucleotide expression constructs, vectors, host cells to treat or prevent Chlamydia infections in an animal.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited May 30, 2006), and these tables can be adapted in a number of ways. See Nakamura, Y., et al. “Codon usage tabulated from the international DNA sequence databases: status for the year 2000” Nucl. Acids Res. 28:292 (2000). Codon usage tables for humans and Escherichia coli, calculated from GenBank Release 151.0, are reproduced below as Tables 2-4 (from http://www.kazusa.or.jp/codon/ supra). These tables use mRNA nomenclature, and so instead of thymine (T) which is found in DNA, the tables use uracil (U) which is found in RNA. The tables have been adapted so that frequencies are calculated for each amino acid, rather than for all 64 codons.

TABLE 2
Codon Usage Table for Human Genes (Homo sapiens)

| Amino Acid | Codon | Frequency |
|---|---|---|
| Phe | UUU | 0.4525 |
| | UUC | 0.5475 |
| Leu | UUA | 0.0728 |
| | UUG | 0.1266 |
| | CUU | 0.1287 |
| | CUC | 0.1956 |
| | CUA | 0.0700 |
| | CUG | 0.4062 |
| Ile | AUU | 0.3554 |
| | AUC | 0.4850 |
| | AUA | 0.1596 |
| Met | AUG | 1.0000 |
| Val | GUU | 0.1773 |
| | GUC | 0.2380 |
| | GUA | 0.1137 |
| | GUG | 0.4710 |
| Ser | UCU | 0.1840 |
| | UCC | 0.2191 |
| | UCA | 0.1472 |
| | UCG | 0.0565 |
| | AGU | 0.1499 |
| | AGC | 0.2433 |
| Pro | CCU | 0.2834 |
| | CCC | 0.3281 |
| | CCA | 0.2736 |
| | CCG | 0.1149 |
| Thr | ACU | 0.2419 |
| | ACC | 0.3624 |
| | ACA | 0.2787 |
| | ACG | 0.1171 |
| Ala | GCU | 0.2637 |
| | GCC | 0.4037 |
| | GCA | 0.2255 |
| | GCG | 0.1071 |
| Tyr | UAU | 0.4347 |
| | UAC | 0.5653 |
| His | CAU | 0.4113 |
| | CAC | 0.5887 |
| Gln | CAA | 0.2541 |
| | CAG | 0.7459 |
| Asn | AAU | 0.4614 |
| | AAC | 0.5386 |
| Lys | AAA | 0.4212 |
| | AAG | 0.5788 |
| Asp | GAU | 0.4613 |
| | GAC | 0.5387 |
| Glu | GAA | 0.4161 |
| | GAG | 0.5839 |
| Cys | UGU | 0.4468 |
| | UGC | 0.5532 |
| Trp | UGG | 1.0000 |
| Arg | CGU | 0.0830 |
| | CGC | 0.1927 |
| | CGA | 0.1120 |
| | CGG | 0.2092 |
| | AGA | 0.2021 |
| | AGG | 0.2011 |
| Gly | GGU | 0.1632 |
| | GGC | 0.3438 |
| | GGA | 0.2459 |
| | GGG | 0.2471 |

TABLE 3
Codon Usage Table for Escherichia Coli.

| Amino Acid | Codon | Frequency of usage |
|---|---|---|
| Phe | UUU | 0.51 |
| | UUC | 0.49 |
| Leu | UUA | 0.11 |
| | UUG | 0.11 |
| | CUU | 0.10 |
| | CUC | 0.10 |
| | CUA | 0.03 |
| | CUG | 0.55 |
| Ile | AUU | 0.47 |
| | AUC | 0.46 |
| | AUA | 0.07 |
| Met | AUG | 1.00 |
| Val | GUU | 0.29 |
| | GUC | 0.20 |
| | GUA | 0.17 |
| | GUG | 0.34 |
| Ser | UCU | 0.19 |
| | UCC | 0.17 |
| | UCA | 0.12 |
| | UCG | 0.13 |
| | AGU | 0.13 |
| | AGC | 0.27 |
| Pro | CCU | 0.16 |
| | CCC | 0.10 |
| | CCA | 0.20 |
| | CCG | 0.55 |
| Thr | ACU | 0.21 |
| | ACC | 0.43 |
| | ACA | 0.30 |
| | ACG | 0.23 |
| Ala | GCU | 0.19 |
| | GCC | 0.25 |
| | GCA | 0.22 |
| | GCG | 0.34 |
| Tyr | UAU | 0.53 |
| | UAC | 0.47 |
| His | CAU | 0.52 |
| | CAC | 0.48 |
| Gln | CAA | 0.31 |
| | CAG | 0.69 |
| Asn | AAU | 0.39 |
| | AAC | 0.61 |
| Lys | AAA | 0.76 |
| | AAG | 0.24 |
| Asp | GAU | 0.59 |
| | GAC | 0.41 |
| Glu | GAA | 0.70 |
| | GAG | 0.30 |
| Cys | UGU | 0.40 |
| | UGC | 0.60 |
| Trp | UGG | 1.00 |
| Arg | CGU | 0.42 |
| | CGC | 0.37 |
| | CGA | 0.05 |
| | CGG | 0.08 |
| | AGA | 0.04 |
| | AGG | 0.03 |
| Gly | GGU | 0.38 |
| | GGC | 0.40 |
| | GGA | 0.09 |
| | GGG | 0.13 |

TABLE 4
Codon Usage Table for P. Fluorescens.

| Amino Acid | Codon | Frequency of usage |
|---|---|---|
| Phe | UUU | 0.27 |
| | UUC | 0.73 |
| Leu | UUA | 0.02 |
| | UUG | 0.19 |
| | CUU | 0.06 |
| | CUC | 0.15 |
| | CUA | 0.02 |
| | CUG | 0.56 |
| Ile | AUU | 0.23 |
| | AUC | 0.72 |
| | AUA | 0.04 |
| Met | AUG | 1.00 |
| Val | GUU | 0.11 |
| | GUC | 0.31 |
| | GUA | 0.09 |
| | GUG | 0.49 |
| Ser | UCU | 0.05 |
| | UCC | 0.19 |
| | UCA | 0.06 |
| | UCG | 0.23 |
| | AGU | 0.10 |
| | AGC | 0.37 |
| Pro | CCU | 0.13 |
| | CCC | 0.26 |
| | CCA | 0.12 |
| | CCG | 0.49 |
| Thr | ACU | 0.10 |
| | ACC | 0.61 |
| | ACA | 0.07 |
| | ACG | 0.21 |
| Ala | GCU | 0.10 |
| | GCC | 0.49 |
| | GCA | 0.11 |
| | GCG | 0.30 |
| Tyr | UAU | 0.33 |
| | UAC | 0.67 |
| His | CAU | 0.38 |
| | CAC | 0.62 |
| Gln | CAA | 0.31 |
| | CAG | 0.69 |
| Asn | AAU | 0.26 |
| | AAC | 0.74 |
| Lys | AAA | 0.36 |
| | AAG | 0.64 |
| Asp | GAU | 0.35 |
| | GAC | 0.65 |
| Glu | GAA | 0.52 |
| | GAG | 0.48 |
| Cys | UGU | 0.21 |
| | UGC | 0.79 |
| Trp | UGG | 1.00 |
| Arg | CGU | 0.19 |
| | CGC | 0.51 |
| | CGA | 0.07 |
| | CGG | 0.18 |
| | AGA | 0.02 |
| | AGG | 0.03 |
| Gly | GGU | 0.20 |
| | GGC | 0.59 |
| | GGA | 0.05 |
| | GGG | 0.06 |

By utilizing these or similar tables, one of ordinary skill in the art can apply the frequencies to any given polypeptide sequence, and produce a nucleic acid of a codon-optimized coding region which encodes the polypeptide, but which uses codons optimal for a given species. For example, in some embodiments of the present invention, the coding region is codon-optimized for expression in E. coli.

Codon-optimized coding regions can be designed by various different methods. In one method, a codon usage table is used to find the single most frequent codon used for any given amino acid for a given organism, and that codon is used each time that particular amino acid appears in the polypeptide sequence. For example, referring to Table 2 above for E. coli, for leucine, the most frequent codon is CUG, which is used 55% of the time. Thus all the leucine residues in a given amino acid sequence would be assigned the codon CUG.

Using this method, an E. coli codon-optimized coding region which encodes SEQ ID NO: 2 can be designed. Specifically, the codons are assigned to the coding region encoding SEQ ID NO: 2 as follows: the 33 phenylalanine codons are TTT, the 64 leucine codons are CTG, the 37 isoleucine codons are ATT, the 8 methionine codons are ATG, the 47 valine codons are GTG, the 83 serine codons are AGC, the 34 proline codons are CCG, the 54 threonine codons are ACC, the 72 alanine codons are GCG, the 26 tyrosine codons are TAT, the 6 histidine codons are CAT, the 30 glutamine codons are CAG, the 61 asparagine codons are AAC, the 28 lysine codons are AAA, the 34 aspartic acid codons are GAT, the 23 glutamic acid codons are GAA, the 6 tryptophan codons are TGG, the 17 arginine codons are CGT, the 7 cysteine codons are TGC, and the 87 glycine codons are GGC.

An E. coli codon-optimized coding region which encodes SEQ ID NO:11 can be designed. Specifically, the codons are assigned to the coding region encoding SEQ ID NO:11 as follows: the 56 phenylalanine codons are TTT, the 101 leucine codons are CTG, the 70 isoleucine codons are ATT, the 10 methionine codons are ATG, the 84 valine codons are GTG, the 124 serine codons are AGC, the 28 proline codons are CCG, the 53 threonine codons are ACC, the 121 alanine codons are GCG, the 19 tyrosine codons are TAT, the 21 histidine codons are CAT, the 54 glutamine codons are CAG, the 69 asparagine codons are AAC, the 48 lysine codons are AAA, the 56 aspartic acid codons are GAT, the 86 glutamic acid codons are GAA, the 4 tryptophan codons are TGG, the 34 arginine codons are CGT, the 23 cysteine codons are TGC, and the 151 glycine codons are GGC.

An E. coli codon-optimized coding region which encodes SEQ ID NO:13 can be designed. Specifically, the codons are assigned to the coding region encoding SEQ ID NO:13 as follows: the 30 phenylalanine codons are TTT, the 48 leucine codons are CTG, the 30 isoleucine codons are ATT, the 8 methionine codons are ATG, the 54 valine codons are GTG, the 93 serine codons are AGC, the 34 proline codons are CCG, the 64 threonine codons are ACC, the 61 alanine codons are GCG, the 18 tyrosine codons are TAT, the 3 histidine codons are CAT, the 10 glutamine codons are CAG, the 52 asparagine codons are AAC, the 27 lysine codons are AAA, the 34 aspartic acid codons are GAT, the 26 glutamic acid codons are GAA, the 5 tryptophan codons are TGG, the 15 arginine codons are CGT, the 7 cysteine codons are TGC, and the 83 glycine codons are GGC.

An E. coli codon-optimized coding region which encodes SEQ ID NO:15 can be designed. Specifically, the codons are assigned to the coding region encoding SEQ ID NO:15 as follows: the 27 phenylalanine codons are TTT, the 65 leucine codons are CTG, the 33 isoleucine codons are ATT, the 7 methionine codons are ATG, the 21 valine codons are GTG, the 81 serine codons are AGC, the 26 proline codons are CCG, the 31 threonine codons are ACC, the 43 alanine codons are GCG, the 10 tyrosine codons are TAT, the 15 histidine codons are CAT, the 28 glutamine codons are CAG, the 43 asparagine codons are AAC, the 26 lysine codons are AAA, the 25 aspartic acid codons are GAT, the 31 glutamic acid codons are GAA, the 5 tryptophan codons are TGG, the 15 arginine codons are CGT, the 12 cysteine codons are TGC, and the 38 glycine codons are GGC.

An E. coli codon-optimized coding region which encodes SEQ ID NO:19 can be designed. Specifically, the codons are assigned to the coding region encoding SEQ ID NO:19 as follows: the 10 phenylalanine codons are TTT, the 25 leucine codons are CTG, the 22 isoleucine codons are ATT, the 5 methionine codons are ATG, the 66 valine codons are GTG, the 38 serine codons are AGC, the 29 proline codons are CCG, the 53 threonine codons are ACC, the 37 alanine codons are GCG, the 12 tyrosine codons are TAT, the 8 histidine codons are CAT, the 16 glutamine codons are CAG, the 23 asparagine codons are AAC, the 34 lysine codons are AAA, the 25 aspartic acid codons are GAT, the 31 glutamic acid codons are GAA, the 5 tryptophan codons are TGG, the 20 arginine codons are CGT, the 24 cysteine codons are TGC, and the 34 glycine codons are GGC.

An E. coli codon-optimized coding region which encodes SEQ ID NO:21 can be designed. Specifically, the codons are assigned to the coding region encoding SEQ ID NO:21 as follows: the 4 phenylalanine codons are TTT, the 15 leucine codons are CTG, the 9 isoleucine codons are ATT, the 7 methionine codons are ATG, the 7 valine codons are GTG, the 17 serine codons are AGC, the 6 threonine codons are ACC, the 10 alanine codons are GCG, the 5 tyrosine codons are TAT, the 9 glutamine codons are CAG, the 10 asparagine codons are AAC, the 14 lysine codons are AAA, the 10 aspartic acid codons are GAT, the 18 glutamic acid codons are GAA, the 5 arginine codons are CGT, the 1 cysteine codons is TGC, and the 7 glycine codons are GGC.

In another method, the actual frequencies of the codons are distributed randomly throughout the coding sequence. Thus using this method for optimization, if a hypothetical polypeptide sequence had 100 leucine residues, referring to Table 2 for frequency of usage in the humans, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence will can vary significantly using this method, however, the sequence always encodes the same polypeptide.

When using the previous method, the term “about” is used precisely to account for fractional percentages of codon frequencies for a given amino acid. As used herein, “about” is defined as one amino acid more or one amino acid less than the value given. The whole number value of amino acids is rounded up if the fractional frequency of usage is 0.50 or greater, and is rounded down if the fractional frequency of use is 0.49 or less. Using the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 UUG codons, etc. 12.87 percent of 62 equals 7.98 CUU codons, or “about 8,” i.e., 7, 8, or 9 CUU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

In yet another method, variations of the first two methods listed above can be used. For example, to codon-optimize a polynucleotide sequence for a given host, the two codons used most frequently for a particular amino acid in the given host are identified, and then those two codons are used to encode at least 95% of that amino acid in the sequence of interest. However, the two codons selected for use for that amino acid can then be used at any frequency, independent of the frequency used in the organism. For example, to codon-optimize for E. coli a sequence encoding a hypothetical polypeptide having 62 serine residues, the fractional frequency of codon usage would be calculated by noting that in E. coli, the two most common codons for serine are AGC (27%) and UCU (19%). Thus, either AGC and UCU would be used to encode at least 95% of the serine codons.

Using the above method, another E. coli codon-optimized coding region which encodes SEQ ID NO:2 can be designed. Specifically, the two codons used most frequently for a particular amino acid in E. coli are used at a frequency greater than 95% in the sequence of interest (Table 5, Column A). However, the two codons selected for use for that amino acid can then be used at any frequency, independent of the frequency used in E. coli (Table 5, Columns B, C, D).

TABLE 5

| | | | Frequency of codon usage for codon-optimized | | | |
|---|---|---|---|---|---|---|
| | | Frequency of | coding regions of the invention | | | |
| | Codon | codon usage in E. coli | A | B | C | D |
| Ala | GCG | 34% | 0-100% | 50%-100% | 90%-100% | 93% |
| | GCA | 22% | 0%-5% | 0% | 0% | 0% |
| | GCT | 19% | 0%-5% | 0% | 0% | 0% |
| | GCC | 25% | 0-100% | 0%-50% | 0%-10% | 7% |
| Cys | TGT | 40% | 0%-100% | 50%-100% | 90%-100% | 100% |
| | TGC | 60% | 0%-100% | 0%-50% | 0%-10% | 0% |
| Asp | GAT | 59% | 0%-100% | 50%-100% | 90%-100% | 97% |
| | GAC | 41% | 0%-100% | 0%-50% | 0%-10% | 3% |
| Glu | GAG | 30% | 0%-100% | 0%-50% | 0%-10% | 0% |
| | GAA | 70% | 0%-100% | 50%-100% | 90%-100% | 100% |
| Phe | TTT | 51% | 0%-100% | 0%-50% | 0%-20% | 18% |
| | TTC | 49% | 0%-100% | 50%-100% | 80%-100% | 82% |
| Gly | GGG | 13% | 0%-5% | 0% | 0% | 0% |
| | GGA | 9% | 0%-5% | 0% | 0% | 0% |
| | GGT | 38% | 0%-100% | 50%-100% | 70%-100% | 71% |
| | GGC | 40% | 0%-100% | 0%-50% | 0%-30% | 29% |
| His | CAT | 52% | 0%-100% | 50%-100% | 90%-100% | 100% |
| | CAC | 48% | 0%-100% | 0%-50% | 0% | 0% |
| Ile | ATA | 7% | 0%-5% | 0% | 0% | 0% |
| | ATT | 47% | 0%-100% | 50%-100% | 55%-100% | 59% |
| | ATC | 46% | 0%-100% | 0%-50% | 0%-45% | 41% |
| Lys | AAG | 24% | 0%-100% | 0%-50% | 0%-10% | 0% |
| | AAA | 76% | 0%-100% | 50%-100% | 90%-100% | 100% |
| Leu | TTG | 11% | 0%-5% | 0% | 0% | 0% |
| | TTA | 11% | 0%-100% | 0%-50% | 0%-10% | 0% |
| | CTG | 55% | 0%-100% | 50%-100% | 90%-100% | 100% |
| | CTA | 3% | 0%-5% | 0% | 0% | 0% |
| | CTT | 10% | 0%-5% | 0% | 0% | 0% |
| | CTC | 10% | 0%-5% | 0% | 0% | 0% |
| Met | ATG | 100% | 100% | 100% | 100% | 100% |
| Asn | AAT | 39% | 0%-100% | 0%-50% | 0%-30% | 20% |
| | AAC | 61% | 0%-100% | 50%-100% | 70%-100% | 80% |
| Pro | CCG | 55% | 0%-100% | 50%-100% | 90%-100% | 100% |
| | CCA | 20% | 0%-5% | 0% | 0% | 0% |
| | CCT | 16% | 0%-100% | 0%-50% | 0%-10% | 0% |
| | CCC | 10% | 0%-5% | 0% | 0% | 0% |
| Gln | CAG | 69% | 0%-100% | 50%-100% | 90%-100% | 97% |
| | CAA | 31% | 0%-100% | 0%-50% | 0%-10% | 3% |
| Arg | AGG | 3% | 0%-5% | 0% | 0% | 0% |
| | AGA | 4% | 0%-5% | 0% | 0% | 0% |
| | CGG | 8% | 0%-5% | 0% | 0% | 0% |
| | CGA | 5% | 0%-5% | 0% | 0% | 0% |
| | CGT | 42% | 0%-100% | 50%-100% | 60%-100% | 65% |
| | CGC | 37% | 0%-100% | 0%-50% | 0%-40% | 35% |
| Ser | AGT | 13% | 0%-5% | 0% | 0% | 0% |
| | AGC | 27% | 0%-100% | 40%-100% | 50%-100% | 54% |
| | TCG | 13% | 0%-5% | 0% | 0% | 0% |
| | TCA | 12% | 0%-5% | 0% | 0% | 0% |
| | TCT | 19% | 0%-100% | 0%-60% | 0%-50% | 46% |
| | TCC | 17% | 0%-5% | 0% | 0% | 0% |
| Thr | ACG | 23% | 0%-5% | 0%-5% | 0%-5% | 4% |
| | ACA | 30% | 0%-100% | 0%-50% | 0%-10% | 0% |
| | ACT | 21% | 0%-5% | 0% | 0% | 0% |
| | ACC | 43% | 0%-100% | 50%-100% | 90%-100% | 96% |
| Val | GTG | 34% | 0%-100% | 0%-60% | 0%-50% | 45% |
| | GTA | 17% | 0%-5% | 0% | 0% | 0% |
| | GTT | 29% | 0%-100% | 40%-100% | 50%-100% | 55% |
| | GTC | 20% | 0%-5% | 0% | 0% | 0% |
| Trp | TGG | 100% | 100% | 100% | 100% | 100% |
| Tyr | TAT | 53% | 0%-100% | 40%-100% | 50%-100% | 54% |
| | TAC | 47% | 0%-100% | 50%-100% | 50%-100% | 46% |

Using the above method, in one embodiment, one or more of the codons assigned to the coding region encoding SEQ ID NO:2 are codon optimized as follows: about 65-69 of the 72 alanine codons in the coding region are GCG and about 3-7 of the alanine codons are GCC; about 5-7 of the 7 cysteine codons in the coding region are TGC and about 0-2 of the cysteine codons are TGT; about 31-34 of the 34 aspartic acid codons in the coding region are GAT and about 0-3 of the aspartic acid codons are GAC; about 21-23 of the 23 glutamic acid codons in the coding region are GAA and about 0-2 of the glutamic acid codons are GAG; about 25-29 of the 33 phenylalanine codons in the coding region are TTC and about 4-8 of the phenylalanine codons are TTT; about 60-64 of the 87 glycine codons in the coding region are GGT and about 23-27 of the glycine codons are GGC; about 4-6 of the 6 histidine codons in the coding region are CAT and about 0-2 of the histidine codons are CAC; about 20-24 of the 37 isoleucine codons in the coding region are ATT and about 13-17 of the isoleucine codons are ATC; about 26-28 of the 28 lysine codons in the coding region are AAA and about 0-2 of the lysine codons are AAG; about 62-64 of the 64 leucine codons in the coding region are CTG; about 47-51 of the 61 asparagine codons in the coding region are AAC and about 10-14 of the asparagine codons are AAT; about 32-34 of the 34 proline codons in the coding region are CCG; about 27-30 of the 30 glutamine codons in the coding region are CAG and about 0-3 of the glutamine codons are CAA; about 9-13 of the 17 arginine codons in the coding region are CGT and about 4-8 of the arginine codons are CGC; about 43-47 of the 83 serine codons in the coding region are AGC and about 36-40 of the serine codons are TCT; about 50-54 of the 54 threonine codons in the coding region are ACC and about 0-4 of the threonine codons are ACG; about 24-28 of the 47 valine codons in the coding region are GTT and about 19-23 of the valine codons are GTG; and/or about 12-16 of the 26 tyrosine codons in the coding region are TAT and about 10-14 of the tyrosine codons are TAC.

One or more of the codons assigned to the coding region encoding SEQ ID NO:11 can be codon optimized using the above method as follows: about 109-121 of the 121 alanine codons in the coding region are GCG and about 0-12 of the alanine codons are GCC; about 21-23 of the 23 cysteine codons in the coding region are TGT and about 0-2 of the cysteine codons are TGC; about 50-56 of the 56 aspartic acid codons in the coding region are GAT and about 0-6 of the aspartic acid codons are GAC; about 77-86 of the 86 glutamic acid codons in the coding region are GAG and about 0-9 of the glutamic acid codons are GAA; about 50-56 of the 56 phenylalanine codons in the coding region are TTT and about 0-6 of the phenylalanine codons are TTC; about 136-151 of the 151 glycine codons in the coding region are GGT and about 0-15 of the glycine codons are GGC; about 19-21 of the 21 histidine codons in the coding region are CAT and about 0-2 of the histidine codons are CAC; about 63-70 of the 70 isoleucine codons in the coding region are ATT and about 0-7 of the isoleucine codons are ATC; about 43-48 of the 48 lysine codons in the coding region are AAG and about 0-5 of the lysine codons are AAA; about 91-101 of the 101 leucine codons in the coding region are TTA and about 0-10 leucine codons are CTG; about 62-69 of the 69 asparagine codons in the coding region are AAT and about 0-7 of the asparagine codons are AAC; about 25-28 of the 28 proline codons in the coding region are CCG and about 0-3 of the proline codons are CCA; about 49-54 of the 54 glutamine codons in the coding region are CAG and about 0-5 of the glutamine codons are CAA; about 31-34 of the 34 arginine codons in the coding region are CGT and about 0-3 of the arginine codons are CGC; about 112-124 of the 124 serine codons in the coding region are AGC and about 0-12 of the serine codons are TCT; about 48-53 of the 53 threonine codons in the coding region are ACA and about 0-5 of the threonine codons are ACC; about 76-84 of the 84 valine codons in the coding region are GTG and about 0-8 of the valine codons are GTT; and/or about 17-19 of the 19 tyrosine codons in the coding region are TAT and about 0-2 of the tyrosine codons are TAC.

One or more of the codons assigned to the coding region encoding SEQ ID NO:13 can be codon optimized using the above method as follows: about 55-61 of the 161 alanine codons in the coding region are GCG and about 0-6 of the alanine codons are GCC; about 6-7 of the 7 cysteine codons in the coding region are TGT and about 0-1 of the cysteine codons are TGC; about 31-34 of the 34 aspartic acid codons in the coding region are GAT and about 0-3 of the aspartic acid codons are GAC; about 23-26 of the 26 glutamic acid codons in the coding region are GAG and about 0-3 of the glutamic acid codons are GAA; about 27-30 of the 30 phenylalanine codons in the coding region are TTT and about 0-3 of the phenylalanine codons are TTC; about 75-83 of the 83 glycine codons in the coding region are GGT and about 0-8 of the glycine codons are GGC; about 2-3 of the 3 histidine codons in the coding region are CAT and about 0-1 of the histidine codons are CAC; about 27-30 of the 30 isoleucine codons in the coding region are ATT and about 0-3 of the isoleucine codons are ATC; about 24-27 of the 27 lysine codons in the coding region are AAG and about 0-3 of the lysine codons are AAA; about 43-48 of the 48 leucine codons in the coding region are TTA and about 0-5 leucine codons are CTG; about 47-52 of the 52 asparagine codons in the coding region are AAT and about 0-5 of the asparagine codons are AAC; about 31-34 of the 34 proline codons in the coding region are CCG and about 0-3 of the proline codons are CCA; about 9-10 of the 10 glutamine codons in the coding region are CAG and about 0-1 of the glutamine codons are CAA; about 14-15 of the 15 arginine codons in the coding region are CGT and about 0-1 of the arginine codons are CGC; about 84-93 of the 93 serine codons in the coding region are AGC and about 0-9 of the serine codons are TCT; about 58-64 of the 64 threonine codons in the coding region are ACA and about 0-6 of the threonine codons are ACC; 49-54 of the 54 valine codons in the coding region are GTG and about 0-5 of the valine codons are GTT; and/or about 16-18 of the 18 tyrosine codons in the coding region are TAT and about 0-2 of the tyrosine codons are TAC.

One or more of the codons assigned to the coding region encoding SEQ ID NO:15 can be codon optimized using the above method as follows: about 39-43 of the 43 alanine codons in the coding region are GCG and about 0-4 of the alanine codons are GCC; about 11-12 of the 12 cysteine codons in the coding region are TGT and about 0-1 of the cysteine codons are TGC; about 23-25 of the 25 aspartic acid codons in the coding region are GAT and about 0-2 of the aspartic acid codons are GAC; about 28-31 of the 31 glutamic acid codons in the coding region are GAG and about 0-3 of the glutamic acid codons are GAA; about 24-27 of the 27 phenylalanine codons in the coding region are TTT and about 0-3 of the phenylalanine codons are TTC; about 34-38 of the 38 glycine codons in the coding region are GGT and about 0-4 of the glycine codons are GGC; about 14-15 of the 15 histidine codons in the coding region are CAT and about 0-1 of the histidine codons are CAC; about 30-33 of the 33 isoleucine codons in the coding region are ATT and about 0-3 of the isoleucine codons are ATC; about 23-26 of the 26 lysine codons in the coding region are AAG and about 0-3 of the lysine codons are AAA; about 57-65 of the 65 leucine codons in the coding region are TTA and about 0-7 leucine codons are CTG; about 39-43 of the 43 asparagine codons in the coding region are AAT and about 0-4 of the asparagine codons are AAC; about 23-26 of the 26 proline codons in the coding region are CCG and about 0-3 of the proline codons are CCA; about 25-28 of the 28 glutamine codons in the coding region are CAG and about 0-3 of the glutamine codons are CAA; about 14-15 of the 15 arginine codons in the coding region are CGT and about 0-1 of the arginine codons are CGC; about 73-81 of the 81 serine codons in the coding region are AGC and about 0-8 of the serine codons are TCT; about 28-31 of the 31 threonine codons in the coding region are ACA and about 0-3 of the threonine codons are ACC; 19-21 of the 21 valine codons in the coding region are GTG and about 0-2 of the valine codons are GTT; and/or about 9-10 of the 10 tyrosine codons in the coding region are TAT and about 0-1 of the tyrosine codons are TAC.

One or more of the codons assigned to the coding region encoding SEQ ID NO:19 can be codon optimized using the above method as follows: about 33-37 of the 37 alanine codons in the coding region are GCG and about 0-4 of the alanine codons are GCC; about 22-24 of the 24 cysteine codons in the coding region are TGT and about 0-2 of the cysteine codons are TGC; about 23-25 of the 25 aspartic acid codons in the coding region are GAT and about 0-3 of the aspartic acid codons are GAC; about 28-31 of the 31 glutamic acid codons in the coding region are GAG and about 0-3 of the glutamic acid codons are GAA; about 9-10 of the 10 phenylalanine codons in the coding region are TTT and about 0-1 of the phenylalanine codons are TTC; about 31-34 of the 33 glycine codons in the coding region are GGT and about 0-3 of the glycine codons are GGC; about 7-8 of the 8 histidine codons in the coding region are CAT and about 0-1 of the histidine codons are CAC; about 20-22 of the 22 isoleucine codons in the coding region are ATT and about 0-2 of the isoleucine codons are ATC; about 31-34 of the 34 lysine codons in the coding region are AAG and about 0-3 of the lysine codons are AAA; about 22-25 of the 25 leucine codons in the coding region are TTA and about 0-3 leucine codons are CTG; about 21-23 of the 23 asparagine codons in the coding region are AAT and about 0-2 of the asparagine codons are AAC; about 26-29 of the 29 proline codons in the coding region are CCG and about 0-3 of the proline codons are CCA; about 14-16 of the 16 glutamine codons in the coding region are CAG and about 0-2 of the glutamine codons are CAA; about 18-20 of the 20 arginine codons in the coding region are CGT and about 0-2 of the arginine codons are CGC; about 34-38 of the 38 serine codons in the coding region are AGC and about 0-4 of the serine codons are TCT; about 48-53 of the 53 threonine codons in the coding region are ACA and about 0-5 of the threonine codons are ACC; 59-66 of the 66 valine codons in the coding region are GTG and about 0-7 of the valine codons are GTT; and/or about 11-12 of the 12 tyrosine codons in the coding region are TAT and about 0-1 of the tyrosine codons are TAC.

One or more of the codons assigned to the coding region encoding SEQ ID NO:21 can be codon optimized using the above method as follows: about 9-10 of the 10 alanine codons in the coding region are GCG and about 0-1 of the alanine codons are GCC; the cysteine codon in the coding region is TGT; about 9-10 of the 10 aspartic acid codons in the coding region are GAT and about 0-1 of the aspartic acid codons are GAC; about 16-18 of the 18 glutamic acid codons in the coding region are GAG and about 0-2 of the glutamic acid codons are GAA; about 3-4 of the 4 phenylalanine codons in the coding region are TTT and about 0-1 of the phenylalanine codons are TTC; about 6-7 of the 7 glycine codons in the coding region are GGT and about 0-1 of the glycine codons are GGC; about 8-9 of the 9 isoleucine codons in the coding region are ATT and about 0-1 of the isoleucine codons are ATC; about 13-14 of the 14 lysine codons in the coding region are AAG and about 0-1 of the lysine codons are AAA; about 14-15 of the 15 leucine codons in the coding region are TTA and about 0-1 leucine codons are CTG; about 9-10 of the 10 asparagine codons in the coding region are AAT and about 0-1 of the asparagine codons are AAC; about 8-9 of the 9 glutamine codons in the coding region are CAG and about 0-1 of the glutamine codons are CAA; about 4-5 of the 5 arginine codons in the coding region are CGT and about 0-1 of the arginine codons are CGC; about 15-17 of the 17 serine codons in the coding region are AGC and about 0-2 of the serine codons are TCT; about 5-6 of the 6 threonine codons in the coding region are ACA and about 0-1 of the threonine codons are ACC; 6-7 of the 7 valine codons in the coding region are GTG and about 0-1 of the valine codons are GTT; and/or about 4-5 of the 5 tyrosine codons in the coding region are TAT and about 0-1 of the tyrosine codons are TAC.

In some embodiments, the E. coli-optimized coding region encoding SEQ ID NO:2 comprises a nucleotide sequence wherein: about 67 of the 72 alanine codons in the coding region are GCG and about 5 of the alanine codons are GCC; about 7 of the 7 cysteine codons in the coding region are TGC; about 33 of the 34 aspartic acid codons in the coding region are GAT and about 1 of the aspartic acid codons are GAC; about 23 of the 23 glutamic acid codons in the coding region are GAA; about 27 of the 33 phenylalanine codons in the coding region are TTC and about 6 of the phenylalanine codons are TTT; about 62 of the 87 glycine codons in the coding region are GGT and about 25 of the glycine codons are GGC; about 6 of the 6 histidine codons in the coding region are CAT; about 22 of the 37 isoleucine codons in the coding region are ATT and about 15 of the isoleucine codons are ATC; about 28 of the 28 lysine codons in the coding region are AAA; about 64 of the 64 leucine codons in the coding region are CTG; about 49 of the 61 asparagine codons in the coding region are AAC and about 12 of the asparagine codons are AAT; about 34 of the 34 proline codons in the coding region are GAT; about 29 of the 30 glutamine codons in the coding region are CAG and about 1 of the glutamine codons are CAA; about 11 of the 17 arginine codons in the coding region are CGT and about 6 of the arginine codons are CGC; about 45 of the 83 serine codons in the coding region are AGC and about 38 of the serine codons are TCT; about 52 of the 54 threonine codons in the coding region are ACC and about 2 of the threonine codons are ACG; about 26 of the 47 valine codons in the coding region are GTT and about 21 of the valine codons are GTG; and about 14 of the 26 tyrosine codons in the coding region are TAT and about 12 of the tyrosine codons are TAC.

A representative E. coli codon-optimized coding region as described above encoding SEQ ID NO:2, is exemplified in a nucleic acid comprising SEQ ID NO:3. One of skill in the art could apply the same methodology used for E. coli in Table 5 and apply it to optimize codon usage for any other organism (e.g., Salmonella enterica serovars such as S. typhi and S. typhimurium) in which frequency is known or can be determined by methods known in the art.

For instance, using a combination of codon-optimization techniques as described above, a P. fluorescens codon-optimized coding region can also be designed. Specifically, the two codons used most frequently for a particular amino acid in P. fluorescens can be used at a frequency greater than 95% in the sequence of interest (Table 6, Column A). However, the two codons selected for use for that amino acid can be used at any frequency, independent of the frequency used in P. fluorescens (Table 6, Columns B, C, D).

TABLE 6

| | | Frequency | Frequency of codon usage for codon-optimized | | | |
|---|---|---|---|---|---|---|
| | | of codon | coding regions of the invention | | | |
| | Codon | usage in P. fluorescens | A | B | C | D |
| Ala | GCG | 30% | 0-100% | 50%-100% | 90%-100% | 95% |
| | GCA | 11% | 0%-5% | 0% | 0% | 0% |
| | GCT | 10% | 0%-5% | 0% | 0% | 0% |
| | GCC | 49% | 0-100% | 0%-50% | 0%-10% | 5% |
| Cys | TGT | 21% | 0%-100% | 50%-100% | 90%-100% | 100% |
| | TGC | 79% | 0%-100% | 0%-50% | 0%-10% | 0% |
| Asp | GAT | 35% | 0%-100% | 50%-100% | 90%-100% | 95% |
| | GAC | 65% | 0%-100% | 0%-50% | 0%-10% | 5% |
| Glu | GAG | 52% | 0%-100% | 0%-50% | 0%-10% | 0% |
| | GAA | 48% | 0%-100% | 50%-100% | 90%-100% | 100% |
| Phe | TTT | 27% | 0%-100% | 0%-50% | 0%-20% | 20% |
| | TTC | 73% | 0%-100% | 50%-100% | 80%-100% | 80% |
| Gly | GGG | 6% | 0%-5% | 0% | 0% | 0% |
| | GGA | 5% | 0%-5% | 0% | 0% | 0% |
| | GGT | 20% | 0%-100% | 50%-100% | 70%-100% | 70% |
| | GGC | 59% | 0%-100% | 0%-50% | 0%-30% | 30% |
| His | CAT | 38% | 0%-100% | 50%-100% | 90%-100% | 100% |
| | CAC | 62% | 0%-100% | 0%-50% | 0% | 0% |
| Ile | ATA | 4% | 0%-5% | 0% | 0% | 0% |
| | ATT | 23% | 0%-100% | 50%-100% | 55%-100% | 60% |
| | ATC | 72% | 0%-100% | 0%-50% | 0%-45% | 40% |
| Lys | AAG | 64% | 0%-100% | 0%-50% | 0%-10% | 0% |
| | AAA | 36% | 0%-100% | 50%-100% | 90%-100% | 100% |
| Leu | TTG | 19% | 0%-100% | 0%-50% | 0%-10% | 0% |
| | TTA | 2% | 0%-5% | 0% | 0% | 0% |
| | CTG | 56% | 0%-100% | 50%-100% | 90%-100% | 100% |
| | CTA | 2% | 0%-5% | 0% | 0% | 0% |
| | CTT | 6% | 0%-5% | 0% | 0% | 0% |
| | CTC | 15% | 0%-5% | 0% | 0% | 0% |
| Met | ATG | 100% | 100% | 100% | 100% | 100% |
| Asn | AAT | 26% | 0%-100% | 0%-50% | 0%-30% | 20% |
| | AAC | 74% | 0%-100% | 50%-100% | 70%-100% | 80% |
| Pro | CCG | 49% | 0%-100% | 50%-100% | 90%-100% | 100% |
| | CCA | 12% | 0%-5% | 0% | 0% | 0% |
| | CCT | 13% | 0%-5% | 0% | 0% | 0% |
| | CCC | 26% | 0%-100% | 0%-50% | 0%-10% | 0% |
| Gln | CAG | 69% | 0%-100% | 50%-100% | 90%-100% | 95% |
| | CAA | 31% | 0%-100% | 0%-50% | 0%-10% | 5% |
| Arg | AGG | 3% | 0%-5% | 0% | 0% | 0% |
| | AGA | 2% | 0%-5% | 0% | 0% | 0% |
| | CGG | 18% | 0%-5% | 0% | 0% | 0% |
| | CGA | 7% | 0%-5% | 0% | 0% | 0% |
| | CGT | 19% | 0%-100% | 50%-100% | 60%-100% | 65% |
| | CGC | 51% | 0%-100% | 0%-50% | 0%-40% | 35% |
| Ser | AGT | 10% | 0%-5% | 0% | 0% | 0% |
| | AGC | 37% | 0%-100% | 40%-100% | 50%-100% | 60% |
| | TCG | 23% | 0%-100% | 0%-60% | 0%-50% | 40% |
| | TCA | 6% | 0%-5% | 0% | 0% | 0% |
| | TCT | 5% | 0%-5% | 0% | 0% | 0% |
| | TCC | 19% | 0%-5% | 0% | 0% | 0% |
| Thr | ACG | 21% | 0%-5% | 0%-5% | 0%-5% | 5% |
| | ACA | 7% | 0%-100% | 0%-50% | 0%-10% | 0% |
| | ACT | 10% | 0%-5% | 0% | 0% | 0% |
| | ACC | 61% | 0%-100% | 50%-100% | 90%-100% | 95% |
| Val | GTG | 49% | 0%-100% | 0%-60% | 0%-50% | 45% |
| | GTA | 9% | 0%-5% | 0% | 0% | 0% |
| | GTT | 11% | 0%-5% | 0% | 0% | 0% |
| | GTC | 31% | 0%-100% | 40%-100% | 50%-100% | 55% |
| Trp | TGG | 100% | 100% | 100% | 100% | 100% |
| Tyr | TAT | 33% | 0%-100% | 40%-100% | 50%-100% | 55% |
| | TAC | 67% | 0%-100% | 50%-100% | 50%-100% | 45% |

As described above, the term “about” means that the number of amino acids encoded by a certain codon may be one more or one less than the number given. It would be understood by those of ordinary skill in the art that the total number of any amino acid in the polypeptide sequence must remain constant, therefore, if there is one “more” of one codon encoding a give amino acid, there would have to be one “less” of another codon encoding that same amino acid.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. Constructing a rudimentary algorithm to assign codons based on a given frequency can also easily be accomplished with basic mathematical functions by one of ordinary skill.

Codon placement in a polynucleotide at an optimized frequency to encode a given polypeptide sequence by any of the methods described herein may be varied to account for cloning or expression issues. For example, a codon may be assigned to a particular amino acid so as to create or destroy a restriction enzyme cleavage site. Creation or destruction of restriction enzyme sites may facilitate DNA manipulation by assisting with cloning or forming identifying markers. Alternatively, a codon may be assigned to a particular amino acid so as to achieve a desired secondary structure of the polynucleotide or remove an unwanted secondary structure.

In certain embodiments, an entire polypeptide sequence, or fragment, variant, or derivative thereof is codon optimized by any of the methods described herein or by other methods. Various desired fragments, variants or derivatives are designed, and each is then codon-optimized individually. In addition, partially codon-optimized coding regions of the present invention can be designed and constructed. For example, the invention includes a nucleic acid of a codon-optimized coding region encoding a polypeptide in which at least about 1%, 2%, 3,% 4%, 5%, 10%, 15%, 20%, 25%, 30%, 35%, 40%, 45%, 50%, 55%, 60%, 65%, 70%, 75%, 80%, 85%, 90%, 95%, or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence.

In additional embodiments, a full-length polypeptide sequence is codon-optimized for a given species resulting in a codon-optimized coding region encoding the entire polypeptide, and then nucleic acids of the codon-optimized coding region, which encode fragments, variants, and derivatives of the polypeptide are made from the original codon-optimized coding region. As would be well understood by those of ordinary skill in the art, if codons have been randomly assigned to the full-length coding region based on their frequency of use in a given species, nucleic acids encoding fragments, variants, and derivatives would not necessarily be fully codon optimized for the given species. However, such sequences are still much closer to the codon usage of the desired species than the native codon usage. The advantage of this approach is that synthesizing codon-optimized nucleic acids encoding each fragment, variant, and derivative of a given polypeptide, although routine, would be time consuming and would result in significant expense.

The present invention provides isolated polynucleotides comprising codon-optimized coding regions of a polypeptide comprising at least about 70%, 75%, 80%, 85%, 90%, 95%, 96%, 97%, 98%, or 99% identity to any one of SEQ ID NOS: 2, 11, 13, 19, or 21 or amino acids 42-743 of SEQ ID NO: 29 or amino acids 64-765 of SEQ ID NO: 32, or fragments, variants, or derivatives thereof, where the polypeptide is soluble in the absence of denaturing agents. In certain embodiments described herein, a codon-optimized coding region encoding any one of SEQ ID NOS: 2, 11, 13, 19, or 21, is optimized according to codon usage in E. coli. Alternatively, a codon-optimized coding region encoding any one of SEQ ID NOS: 2, 11, 13, 19, or 21 or amino acids 42-743 of SEQ ID NO: 29 or amino acids 64-765 of SEQ ID NO: 32, may be optimized according to codon usage in any plant, animal, or microbial species, e.g., bacteria such as E. coli, S. enterica, Pseudomonas aeruginosa or Pseudomonas fluorescens; fungi such as yeast; and mammals such as humans, rats, mouse, primates, or rabbits.

In certain embodiments, the present invention provides an isolated nucleic acid which encodes at least 10, at least 20, at least 30, at least 40, at least 50, at least 60, at least 70, at least 80, at least 90, at least 95, or at least 100 or more contiguous amino acids of any one of SEQ ID NOS: 2, 11, 13, 19, or 21 or amino acids 42-743 of SEQ ID NO: 29 or amino acids 64-765 of SEQ ID NO: 32, wherein the continguous amino acids are soluble in the absence of denaturing agents. For instance, the invention includes an isolated nucleic acid which encodes at least 10, at least 20, at least 30, at least 40, at least 50, at least 60, at least 70, at least 80, at least 90, at least 95, or at least 100 or more contiguous amino acids of any one of SEQ ID NOS: 2, 11, 13, 19, or 21 or amino acids 42-743 of SEQ ID NO: 29 or amino acids 64-765 of SEQ ID NO: 32, where the nucleic acid is a fragment of a codon-optimized coding region encoding any one of SEQ ID NOS: 2, 11, 13, 19, or 21, and wherein the contiguous amino acids are soluble in the absence of denaturing agents.

In certain embodiments, the present invention provides an isolated nucleic acid which encodes a polypeptide comprising at least about 70%, 75%, 80%, 85%, 90%, 91%, 92%, 93%, 94%, 95%, 96%, 97%, 98%, or 99% identity to CT84 (SEQ ID NO: 2), PmpD-133 (SEQ ID NO: 11), PmpH-78 (SEQ ID NO:13), OmcB-1 (SEQ ID NO: 19), or OmpH-1 (SEQ ID NO: 21), and where the nucleic acid is a variant of a codon-optimized coding region encoding one of SEQ ID NOS: 2, 11, 13, 19 or 21 respectively, and wherein the polypeptide is soluble in the absence of denaturing agents.

### DNA Synthesis

A number of options are available for synthesizing codon optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides is designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

### DNA Hybridization

A nucleotide sequence encoding a polypeptide comprising at least about 70%, 75%, 80%, 85%, 90%, 95%, 96%, 97%, 98%, or 99% identity to one of SEQ ID NOS: 2, 11, 13, 19, or 21 is useful for its ability to hybridize selectively, i.e., form duplex molecules with complementary stretches of other polypeptide genes. Depending on the application, a variety of hybridization conditions may be employed to achieve varying sequence identities. In specific aspects, nucleic acids are provided which comprise a sequence complementary to at least 10, 15, 25, 50, 100, 200 or 250 nucleotides of the CT84 polypeptide gene. In specific embodiments, nucleic acids which hybridize to a CT84 protein nucleic acid (e.g. having sequence SEQ ID NO: 1 or 3) under annealing conditions of low, moderate or high stringency conditions are within the scope of the invention. In some embodiments, nucleic acids which hybridize to any one of SEQ ID NOS: 10, 12, 18, or 20 under annealing conditions of low, moderate or high stringency conditions are within the scope of the invention.

For a high degree of selectivity, relatively stringent conditions are used to form the duplexes, such as, by way of example and not limitation, low salt and/or high temperature conditions, such as provided by hybridization in a solution of salt, e.g., 0.02 M to 0.15 M NaCl at temperatures of between about 50° C. to 70° C. For some applications, less stringent hybridization conditions are required, by way of example and not limitation, such as provided by hybridization in a solution of 0.15 M to 0.9 M salt, e.g., NaCl, at temperatures ranging from between about 20° C. to 55° C. Hybridization conditions can also be rendered more stringent by the addition of increasing amounts of formamide, to destabilize the hybrid duplex. Thus, particular hybridization conditions can be readily manipulated. By way of example and not limitation, in general, convenient hybridization temperatures in the presence of 50% formamide are: 42° C. for a probe which is 95% to 100% homologous to the target fragment, 37° C. for 90% to 95% homology and 32° C. for 70% to 90% homology. One aspect of the invention is directed to an isolated nucleic acid which hybridizes, upon incubation in a solution comprising 50% formamide at about 37° C., to a DNA sequence which is complementary to any one of SEQ ID NO: 1, SEQ ID NO: 3, or a polynucleotide that is a codon-optimized coding region encoding a polypeptide of SEQ ID NO: 2, wherein the nucleic acid encodes a polypeptide which is soluble in the absence of denaturing agents. In some embodiments, the polypeptide is recognized by an antibody that specifically binds to a polypeptide consisting of SEQ ID NO: 2.

In some embodiments, the present invention is directed to an isolated nucleic acid which hybridizes, upon incubation in a solution comprising 50% formamide at about 37° C., to a DNA sequence which is complementary to SEQ ID NO: 10, wherein said nucleic acid encodes a polypeptide which is soluble in the absence of denaturing agents, and wherein said polypeptide is recognized by an antibody that specifically binds to a polypeptide consisting of SEQ ID NO: 11. In some embodiments, the present invention is directed to an isolated nucleic acid which hybridizes, upon incubation in a solution comprising 50% formamide at about 37° C., to a DNA sequence which is complementary to SEQ ID NO: 12, wherein said nucleic acid encodes a polypeptide which is soluble in the absence of denaturing agents, and wherein said polypeptide is recognized by an antibody that specifically binds to a polypeptide consisting of SEQ ID NO: 13. In some embodiments, the present invention is directed to an isolated nucleic acid which hybridizes, upon incubation in a solution comprising 50% formamide at about 37° C., to a DNA sequence which is complementary to SEQ ID NO: 18, wherein said nucleic acid encodes a polypeptide which is soluble in the absence of denaturing agents, and wherein said polypeptide is recognized by an antibody that specifically binds to a polypeptide consisting of SEQ ID NO: 19. In some embodiments, the present invention is directed to an isolated nucleic acid which hybridizes, upon incubation in a solution comprising 50% formamide at about 37° C., to a DNA sequence which is complementary to SEQ ID NO: 20, wherein said nucleic acid encodes a polypeptide which is soluble in the absence of denaturing agents, and wherein said polypeptide is recognized by an antibody that specifically binds to a polypeptide consisting of SEQ ID NO:21.

Other low, moderate and high stringency conditions are well known to those of skill in the art, and will vary predictably depending on the base composition and length of the particular nucleic acid sequence and on the specific organism from which the nucleic acid sequence is derived. For guidance regarding such conditions see, for example, Sambrook et al., Molecular Cloning, A Laboratory Manual, Second Edition, Cold Spring Harbor Press, N.Y., pp. 9.47-9.57 (1989); and Ausubel et al., Current Protocols in Molecular Biology, Green Publishing Associates and Wiley Interscience, N.Y. (1989) both of which are incorporate herein, by reference.

### Vectors and Expression Systems

The present invention further provides a vector comprising a polynucleotide of the present invention. The vector can be, for example, in the form of a plasmid, a viral particle, a phage, cosmid, etc. A polynucleotide of the present invention may be in a circular or linearized plasmid or vector, or other linear DNA which may also be non-infectious and nonintegrating (i.e., does not integrate into the genome of host cells). Procedures for inserting a nucleotide sequence into an expression vector, and transforming or transfecting into an appropriate host cell and cultivating under conditions suitable for expression are generally known in the art, as described generally in Sambrook et al., Molecular Cloning, a Laboratory Manual, Cold Spring Harbor Press, Cold Spring Harbor, N.Y. (1989).

In accordance with one aspect of the present invention, there is provided a vector comprising a nucleic acid, where the nucleic acid is a fragment of a codon-optimized coding region operably encoding a polypeptide comprising at least about 70%, 75%, 80%, 85%, 90%, 95%, 96%, 97%, 98%, or 99% identity to SEQ ID NO: 2. Additional Chlamydia-derived coding or non-coding regions may also be included on a vector, e.g., a plasmid, gene expression cassette or on a separate vector, and expressed, either using native Chlamydia codons or codons optimized for expression in the host in which the polypeptide is being expressed. When such a vector is delivered to a host, e.g., to a bacterial, plant or eukaryotic cell, or alternatively, in vivo to a tissue of the animal to be treated or immunized, the transcriptional unit will thus express the encoded gene product. The level of expression of the gene product will depend to a significant extent on the strength of the associated promoter and the presence and activation of an associated enhancer element, as well as the optimization of the coding region.

A variety of host-expression vector systems may be utilized to express the polypeptides of the present invention. Vector-host systems include, but are not limited to, systems such as bacterial, mammalian, yeast, insect or plant cell systems, either in vivo, e.g., in an animal or in vitro, e.g., in mammalian cell cultures. The selection of an appropriate host is deemed to be within the scope of those skilled in the art from the teachings herein.

Host cells are genetically engineered (transduced or transformed or transfected) with the vectors of this invention as described above. Thus, one aspect of the invention is directed to a host cell comprising a vector which contains a polynucleotide of the present invention. The engineered host cell can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

Bacterial host-expression vector systems include, but are not limited to, a prokaryote (e.g., E. coli (e.g., BL21, BL21(DE3), BL21(DE3)pLysS strains), Bacillus subtilis, Salmonella typhimurium and various species within the genera Pseudomonas, e.g., P. aeruginosa or P. fluorescens (e.g. PFēnex™ (Dowpharma)), Streptomyces spp., or Staphylococcus spp.) transformed with recombinant bacteriophage DNA, plasmid DNA or cosmid DNA expression vectors containing polypeptide coding regions of the present invention. In some embodiments, the PFēnex™ system is used. The PFēnex™ expression system utilizes P. fluorescens biovar I, designated MB101, and compatible plasmids. In some embodiments, the plasmids used with P. fluorescens use the tac promoter system regulated by the LacI protein via IPTG induction. In some embodiments, the bacterial host can have a auxotrophic chromosomal deletion, e.g., pyrF, in which the deletion is complemented by the vector, to alleviate the need for antibiotic-resistance genes. A large number of suitable vectors are known to those of skill in the art, and are commercially available. The following bacterial vectors are provided by way of example: pET, pET-43.1 (Novagen), pET15b (Novagen), pQE70, pQE60, pQE-9 (Qiagen), phagescript, psiX174, pBluescript SK, pbsks, pNH8A, pNH16a, pNH18A, pNH46A (Stratagene), ptrc99a, pKK223-3, pKK233-3, pDR540, pBR322, pPS10, RSF1010, pRSF2 (Novagen), pRIT5 (Pharmacia); pCR (Invitrogen); and pLex (Invitrogen). However, any other plasmid or vector can be used as long as it is replicable and viable in the host. In some embodiments, the expression vector comprises the plasmid pLex. The pLex plasmid comprises a multiple cloning site that is tightly regulated by a tryptophan-inducible expression system utilizing the strong P_{L} promoter from bacteriophage lambda, and the cI repressor protein. This pLex expression vector is especially useful for the expression of potentially toxic proteins in E. coli. In addition, the lambda promoter provides high-level expression of recombinant proteins.

In one embodiment of the invention, the nucleic acid is cloned into a gene expression cassette such as is described in WO 00/14240 which is herein incorporated by reference in its entirety. In one embodiment of the invention, the gene expression cassette is integrated into the host cell genome, for instance, by homologous recombination.

Generally, bacterial vectors will include origins of replication and selectable markers, e.g., the ampicillin, tetracycline, kanamycin, resistance genes of E. coli, permitting transformation of the host cell and a promoter derived from a highly-expressed gene to direct transcription of a downstream structural sequence. Such promoters include, but are not limited to, the T7 promoter, lambda (λ) promoter, T5 promoter, T4 promoter, and lac promoter, or promoters derived from operons encoding glycolytic enzymes such as 3-phosphoglycerate kinase (PGK), acid phosphatase, or heat shock proteins, among others.

Once an expression vector is selected, the polynucleotide of the invention is cloned downstream of the promoter, often in a polylinker region. This plasmid is transformed into an appropriate bacterial strain, and DNA is prepared using standard techniques. The orientation and DNA sequence of the polynucleotide as well as all other elements included in the vector, are confirmed using restriction mapping, DNA sequence analysis, and/or PCR analysis. Bacterial cells harboring the correct plasmid can be stored as cell banks.

Examples of mammalian host-expression systems include cell lines capable of expressing a compatible vector, for example, the COS, C127, 3T3, CHO, HeLa and BHK cell lines. Examples of suitable expression vectors include pWLNEO, pSV2CAT, pOG44, pXT1, pSG (Stratagene) pSVK3, pBPV, pMSG, pSVL (Pharmacia), p75.6 (Valentis), pCEP (Invitrogen), and pCEI (Epimmune), as well as viral genomes from which to construct viral vectors such as Simian virus 40 (SV40), bovine papilloma virus, pox virus such as vaccinia virus, and parvovirus, including adeno-associated virus, retrovirus, herpesvirus, adenovirus, retroviral, e.g., murine leukemia virus and lentiviruses (e.g., human immunodeficiency virus), alphavirus, and picornavirus. References citing methods for the in vivo introduction of non-infectious virus genomes to animal tissues are well known to those of ordinary skill in the art.

Generally, mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters may also be derived from viral sources, such as, e.g., human cytomegalovirus (CMV-IE promoter), herpes simplex virus type-1 (HSV TK promoter), the adenovirus late promoter; and the vaccinia virus 7.5K promoter, or can be derived from the genome of mammalian cells (e.g., metallothionein promoter). Nucleic acid sequences derived from the SV40 splice and polyadenylation sites can be used to provide the required nontranscribed genetic elements. A variety of transcription control regions are known to those skilled in the art. These include, without limitation, transcription control regions which function in animal cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from animal genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins). Similarly, a variety of translation control elements are known to those of ordinary skill in the art. These include, but are not limited to ribosome binding sites, translation initiation and termination codons, elements from picornaviruses (particularly an internal ribosome entry site, or IRES, also referred to as a CITE sequence).

Yeast host-expression systems include a yeast host (e.g., Saccharomyces, Pichia, Hansenula, Kluyveromyces, Schizosaccharomyces, Schwanniomyces and Yarrowia) transformed with recombinant yeast expression vectors containing polypeptide coding sequences, employing suitable vectors and control sequences. Suitable yeast expression vectors are known to those in the art and include, but are not limited to, e.g., pAL19, paR3, pBG1, pDBlet, pDB248X, pEA500, pFL20, pIRT2, pJK148, pON163, pSP1, pSP3, pUR19, pART1, pCHY21, REP41, pYZ1N, pSLF104, pSLF172, pDS472, pSGP572, pSLF1072, REP41 MH-N, pFA6a-kanMX6, pARTCM, and pALL.

Insect host systems (e.g., Trichoplusia, Lipidotera, Spodoptera, Drosophila and Sf9) infected with recombinant expression vectors (e.g., baculovirus, pDEST™10 Vector (Invitrogen), pMT-DEST48 Vector (Invitrogen), pFastBac Dual (Invitrogen), pIE1-neo DNA (Novagen), pIEx™-1 DNA (Novagen),) containing polypeptide coding sequences of the present invention are also within the scope of the invention. See e.g., O'Reilly et al., Baculovirus Expression Vectors: A Laboratory Manual. Oxford Univ Press (1994).

Plant cell systems (e.g., Arabidopsis) infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences of the present invention, containing polypeptide coding sequences are also within the scope of the invention. A list of vectors for a wide variety of plants can be found at http://www.arabidopsis.org/servlets/Order?state=catalog (viewed Jun. 20, 2006).

One of skill in the art will recognize that some of the above listed vectors are capable of replicating and expressing polypeptides in more than one type of host, e.g., the pOG44 plasmid can replicate and express polypeptides in both prokaryotic and eukaryotic cells.

### Polypeptides

The present invention is also directed to polypeptides comprising at least 90% identity to any one of SEQ ID NOS: 2, 11, 13, 19, or 21 or amino acids 42-743 of SEQ ID NO: 29 or amino acids 64-765 of SEQ ID NO: 32, wherein the polypeptide is soluble in the absence of denaturing agents. CT84, PmpD-133, PmpH-78, OmcB-1, and OmpH variants are also included in the present invention. For example, polypeptides comprising at least about 70%, 75%, 80%, 85%, 90%, 91%, 92%, 93%, 94%, 95%, 96%, 97%, 98%, or 99% identity to any one of SEQ ID NOS: 2, 11, 13, 19, or 21 or amino acids 42-743 of SEQ ID NO: 29 or amino acids 64-765 of SEQ ID NO: 32, are also within the scope of the invention. In some embodiments, the present invention is directed to a polypeptide comprising at least about 95% identity to any one of SEQ ID NOS: 2, 11, 13, 19, or 21 or amino acids 42-743 of SEQ ID NO: 29 or amino acids 64-765 of SEQ ID NO: 32, wherein the polypeptide is soluble in the absence of denaturing agents. In some embodiments, the present invention is directed to fragments, variants, derivative and analogs of a polypeptide comprising at least about 70%, 75%, 80%, 85%, 90%, 95%, 96%, 97%, 98%, or 99% identity to any one of SEQ ID NOS: 2, 11, 13, 19, or 21 or amino acids 42-743 of SEQ ID NO: 29 or amino acids 64-765 of SEQ ID NO: 32, wherein the polypeptide is soluble in the absence of denaturing agents.

Peptide and polypeptide sequences defined herein are represented by one-letter symbols for amino acid residues as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine).

In some embodiments, the polypeptides of the present invention are isolated. No particular level of purification is required. Recombinantly produced Chlamydia polypeptides and proteins expressed in host cells are considered isolated for purposes of the invention, as are native or recombinant Chlamydia polypeptides which have been separated, fractionated, or partially or substantially purified by any suitable technique, including, but not limited to, by electrophoresis, filtration, chromatography, centrifugation, and the like.

Polypeptides, and fragments, derivatives, analogs, or variants thereof of the present invention can be antigenic and immunogenic Chlamydia polypeptides, which are used to prevent or treat, i.e., cure, ameliorate, lessen the severity of, or prevent or reduce contagion of infectious disease caused by C. trachomatis, C. pneumoniae or other species as disclosed herein.

In certain aspects of the present invention, antigenic epitopes can contain a sequence of at least 4, at least 5, at least 6, at least 7, at least 8, at least 9, at least 10, at least 15, at least 20, at least 25, or between about 15 to about 30 amino acids contained within the amino acid sequence of a polypeptide of the invention. Certain polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Antigenic as well as immunogenic epitopes may be linear, i.e., be comprised of contiguous amino acids in a polypeptide, or may be three dimensional, i.e., where an epitope is comprised of non-contiguous amino acids which come together due to the secondary or tertiary structure of the polypeptide, thereby forming an epitope.

Peptides or polypeptides, e.g., immunogenic epitopes, capable of eliciting an immunogenic response are frequently represented in the primary sequence of a protein, can be characterized by a set of simple chemical rules, and are confined neither to immunodominant regions of intact proteins nor to the amino or carboxyl terminals. Polypeptides that are extremely hydrophobic and those of six or fewer residues generally are ineffective at inducing antibodies, but may still bind antibodies raised against larger portions of the polypeptide; longer peptides, especially those containing proline residues, usually are effective (Sutcliffe, J. G., et al., Science 219:660-666 (1983)).

In some embodiments of the present invention a polypeptide comprising at least about 70%, 75%, 80%, 85%, 90%, 95%, 96%, 97%, 98%, or 99% identity to any one of SEQ ID NOS:2, 11, 13, 19, or 21 or amino acids 42-743 of SEQ ID NO: 29 or amino acids 64-765 of SEQ ID NO: 32 is fused to a heterologous polypeptide. Various heterologous polypeptides can be used which encode their respective heterologous polypeptides. In some embodiments, the heterologous polypeptide is fused to the polypeptide of the present invention. Various heterologous polypeptides can be used, and can be selected from the group consisting of an N- or C-terminal peptide imparting stabilization, secretion, or simplified purification, i.e., His-tag, ubiquitin tag, NusA tag, chitin binding domain, ompT, ompA, pelB, DsbA, DsbC, c-myc, KSI, polyaspartic acid, (Ala-Trp-Trp-Pro)n (SEQ ID NO:10), polyphenyalanine, polycysteine, polyarginine, B-tag, HSB-tag, green fluorescent protein (GFP), hemagglutinin influenza virus (HAI), calmodulin binding protein (CBP), galactose-binding protein, maltose binding protein (MBP), cellulose binding domains (CBD's), dihydrofolate reductase (DHFR), glutathione-S-transferase (GST), streptococcal protein G, staphylococcal protein A, T7gene10, avidinistreptavidin/Strep-tag, trpE, chloramphenicol acetyltransferase, lacZ ((3-Galactosidase), His-patch thioredoxin, thioredoxin, FLAG™ peptide (Sigma-Aldrich), S-tag, and T7-tag. See e.g., Stevens, R. C., Structure, 8:R177-R185 (2000). Other suitable heterologous polypeptides may include other Chlamydia proteins (either native proteins or variants, fragments, or derivatives thereof, e.g., MOMP, PorB, Pmp6, Pmp8, Pmp 11, Pmp20, Pmp21, PmpD, PmpE, PmpG, PmpH, PmpI, OmpH, Omp4, Omp5, Omp85, MIP, OmcA, and OmcB), and in some embodiments, a leader sequence capable of directing secretion of translated protein into the periplasmic space or extracellular medium. In some embodiments, the polypeptide of the present invention can exist as a homopolymer, comprising multiple copies of the same polypeptide.

Production of the polypeptides of the present invention can be achieved by culturing the host cells, expressing the polynucleotides of the present invention, and recovering the polypeptides. Determining conditions for culturing the host cells and expressing the polynucleotide are generally specific to the host cell and the expression system and are within the knowledge of one of skill in the art. Likewise, appropriate methods for recovering the polypeptide of interest are known to those in the art, and include, but are not limited to, electrophoresis (e.g., SDS-PAGE), chromatography, filtration, precipitation, and centrifugation.

### Polypeptide Vaccine Compositions

Vaccines that contain an immunologically effective amount of one or more polypeptides or polynucleotides of the invention are a further embodiment of the invention. Such vaccine compositions may include, for example, lipopeptides (e.g., Vitiello, A. et al., J. Clin. Invest. 95:341, 1995), polypeptides encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995); polypeptide compositions contained in immune stimulating complexes (ISCOMS) (see, e.g., Takahashi et al., Nature 344:873-875, 1990; Hu et al., Clin Exp Immunol. 113:235-243, 1998); multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996); polypeptides expressed from avirulent host cell carriers (e.g., WO 00/068261 and WO 02/072845); particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995); adjuvants (e.g., incomplete freund's advjuvant) (Warren, H. S., Vogel, F. R., and Chedid, L. A. Annu. Rev. Immunol. 4:369, 1986; Gupta, R. K. et al., Vaccine 11:293, 1993); or liposomes (Reddy, R. et al., J. Immunol. 148:1585, 1992; Rock, K. L., Immunol. Today 17:131, 1996).

Compositions of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995), both of which are incorporated herein by reference in their entireties. Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

The concentration of polypeptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

Furthermore, vaccines in accordance with the invention can comprise more than one polypeptide of the invention. For example, in some embodiments a vaccine can comprise two or more of the polypeptides of SEQ ID NOS: 2, 11, 13, 19, and 21 or amino acids 42-743 of SEQ ID NO: 29 or amino acids 64-765 of SEQ ID NO: 32. In some embodiments, the polypeptide vaccine of the present invention can further include a mature polypeptide, or a fragment of a polypeptide, selected from the group consisting of, but not limited to, MOMP, PorB, Pmp6, Pmp8, Pmp11, Pmp20, Pmp21, PmpD, PmpE, PmpG, PmpH, OmpH, Omp4, Omp5, Omp85, MIP, OmcA, and OmcB.

The present invention is also directed to a method of producing a polypeptide vaccine against Chlamydia. In some embodiments, the method of producing the vaccine comprises (a) isolating the polypeptide of the present invention; and (b) adding an adjuvant, carrier and/or excipient to the isolated polypeptide. As the person of ordinary skill in the art would appreciate, the terms “adjuvant,” “carrier,” and “excipient” overlap to a significant extent. For example, a compound which acts as an “adjuvant” may also be a “carrier,” as well as an “excipient,” and certain other compounds normally thought of, e.g., as carriers, may also function as an adjuvant.

In some embodiments, the present invention provides a composition comprising a Chlamydia polypeptide comprising at least about 70%, 75%, 80%, 85%, 90%, 95%, 96%, 97%, 98%, or 99% identity to any one of SEQ ID NOS: 2, 11, 13, 19, or 21 or amino acids 42-743 of SEQ ID NO: 29 or amino acids 64-765 of SEQ ID NO: 32, and a carrier. Carriers that can be used with compositions of the invention are well known in the art, and include, without limitation, e.g., thyroglobulin, albumins such as human serum albumin, tetanus toxoid, polyamino acids such as poly L-lysine, poly L-glutamic acid, influenza, hepatitis B virus core protein, and the like. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the carrier is an immunogenic carrier. Suitably, an immunogenic carrier may be fused to or conjugated to a desired polypeptide or fragment thereof. See, e.g., European Patent No. EP 0385610 B1, which is incorporated herein by reference in its entirety.

Certain compositions can further include one or more adjuvants before, after, or concurrently with the polypeptide. A great variety of materials have been shown to have adjuvant activity through a variety of mechanisms. Potential adjuvants which may be screened for their ability to enhance the immune response according to the present invention include, but are not limited to: inert carriers, such as alum, bentonite, latex, and acrylic particles; pluronic block polymers, such as TiterMax® (block copolymer CRL-8941, squalene (a metabolizable oil) and a microparticulate silica stabilizer), depot formers, such as Freund's adjuvant, surface active materials, such as saponin, lysolecithin, retinal, Quil A, liposomes, and pluronic polymer formulations; macrophage stimulators, such as bacterial lipopolysaccharide; alternate pathway complement activators, such as insulin, zymosan, endotoxin, and levamisole; and non-ionic surfactants, such as poloxamers, poly(oxyethylene)-poly(oxypropylene) tri-block copolymers, cytokines and growth factors; bacterial components (e.g., endotoxins, in particular superantigens, exotoxins and cell wall components); aluminum-based salts such as aluminum hydroxide; calcium-based salts; silica; polynucleotides; toxoids; serum proteins, viruses and virally-derived materials, poisons, venoms, imidazoquiniline compounds, poloxamers, mLT, and cationic lipids. International Patent Application, PCT/US95/09005 incorporated herein by reference describes use of a mutated forms of heat labile toxin of enterotoxigenic E. coli (“mLT”) as an adjuvant. U.S. Pat. No. 5,057,540, incorporated herein by reference, describes the adjuvant, Qs21. In some embodiments, the adjuvant is a toll-like receptor (TLR) stimulating adjuvant. See e.g., Science 312:184-187 (2006). TLR adjuvants include compounds that stimulate the TLRs (e.g., TLR1-TLR13), preferably human TLRs, resulting in an increased immune system response to the vaccine composition of the present invention. TLR adjuvants include, but are not limited to CpG (Coley Pharmaceutical Group Inc.) and MPL (Corixa). In some embodiments, adjuvants include, but are not limited to mLT, CpG, MPL, and aluminum hydroxide. Dosages of the adjuvants can vary according to the specific adjuvants. For example, in some aspects, dosage ranges can include: 10 μg/dose to 500 μg/dose, or 50 μg/dose to 200 μg/dose for CpG. Dosage ranges can include: 2 μg/dose to 100 μg/dose, or 10 μg/dose to 30 μg/dose for MPL. Dosage ranges can include: 10 μg/dose to 500 μg/dose, or 50 μg/dose to 100 μg/dose for aluminum hydroxide. In a prime-boost regimen, as described elsewhere herein, an adjuvant may be used with either the priming immunization, the booster immunization, or both.

In certain adjuvant compositions, the adjuvant is a cytokine. Certain compositions of the present invention comprise one or more cytokines, chemokines, or compounds that induce the production of cytokines and chemokines, or a polynucleotide encoding one or more cytokines, chemokines, or compounds that induce the production of cytokines and chemokines. Examples of cytokines include, but are not limited to granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNω), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th₂ response into a primarily cellular, or Th₁ response. Immune responses to a given antigen may be tested by various immunoassays well known to those of ordinary skill in the art, and/or described elsewhere herein.

The polyeptides of the invention can also be administered via liposome carriers, which serve to target the polypeptides to a particular tissue, such as lymphoid tissue, or to target selectively to infected cells, as well as to increase the half-life of the polypeptide composition. Liposomes include emulsions, foams, micelles, insoluble monolayers, liquid crystals, phospholipid dispersions, lamellar layers and the like. In these preparations, the polypeptide to be delivered is incorporated as part of a liposome, alone or in conjunction with a molecule which binds to a receptor prevalent among lymphoid cells (such as monoclonal antibodies which bind to the CD45 antigen or other costimulatory factor) or with other therapeutic or immunogenic compositions. Thus, liposomes either filled or decorated with a desired polypeptide of the invention can be directed to the site of lymphoid cells, where the liposomes then deliver the polypeptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369. A liposome suspension containing a polypeptide of the invention may be administered intravenously, locally, topically, etc. in a dose which varies according to, inter alia, the manner of administration, the polypeptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more polypeptides of the invention, often at a concentration of 25%-75%.

For aerosol or mucosal administration, the immunogenic polypeptides can be supplied in finely divided form, optionally along with a surfactant, propellant and/or a mucoadhesive, e.g., chitosan. Typical percentages of polypeptides are 0.01%-20% by weight, often 1%-10%. The surfactant must, of course, be pharmaceutically acceptable, and in some embodiments soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, in some embodiments 0.25-5% by weight. The balance of the composition is ordinarily propellant, although an atomizer may be used in which no propellant is necessary and other percentages are adjusted accordingly. In some embodiments, the immunogenic polypeptides can be incorporated within an aerodynamically light particle, such as those particles described in U.S. Pat. No. 6,942,868 or U.S. Pat. Pub. No. 2005/0008633. A carrier can also be included, e.g., lecithin for intranasal delivery.

In some embodiments, the present invention is directed to a multivalent vaccine. For example, a multivalent vaccine of the present invention can comprise a polypeptide comprising at least about 70%, 75%, 80%, 85%, 90%, 95%, 96%, 97%, 98%, or 99% identity to any one of SEQ ID NOS: 2, 11, 13, 19, or 21, wherein the polypeptide is soluble in the absence of denaturing agents, and a polypeptide that elicits an immune reaction to one or more additional organisms and/or viruses, e.g., Haemophilus influenzae type b, Hepatitis B virus, Hepatitis A virus, Hepatitis C virus, Streptococcus pneumoniae, Corynebacterium diphtheriae, Clostridium tetani, Polio virus, Influenza virus, Rubeola virus, Rubella virus, myxovirus, Neisseria, e.g., N. meningitidis, human papilloma virus (HPV), Epstein-Barr virus (EBV), herpes simplex virus, varicella-zoster virus, or other Chlamydia species. In some embodiments, the multivalent vaccine of the present invention can comprise a polypeptide of the present invention and a compatible vaccine, wherein both the vaccine of the present invention and the compatible vaccine are targeted for a similar patient population, e.g., an adolescent population. Examples of multivalent vaccines targeted for a specific patient population include, but are not limited to a vaccine for administration to an adolescent comprising a polypeptide of the present invention and a polypeptide that elicits an immune response to one or more of Hepatitis B virus, Hepatitis C virus, Neisseria, e.g., N. meningitidis, Epstein-Barr virus (EBV), varicella-zoster virus, herpes simplex virus, Streptococcus pneumoniae, human papilloma virus, or other Chlamydia species.

### Polynucleotide Vaccines

In some embodiments, the present invention is also directed to a polynucleotide vaccine. Such polynucleotide vaccine compositions can include those adjuvants, carriers, excipients, or modes of administration listed herein for polypeptide vaccines. In some embodiments, if the adjuvant, carrier, or excipient is a polypeptide, the polynucleotide vaccine composition can further comprise a nucleic acid which encodes the adjuvant, carrier or excipient polypeptide. Polynucleotide vaccine compositions can also include, for example, naked DNA, DNA formulated with PVP, DNA in cationic lipid formulations; DNA encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), viral, bacterial, or, fungal delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990); or particle-absorbed DNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993), etc.

The present invention provides compositions, or polynucleotide vaccines, comprising a polynucleotide encoding a Chlamydia polypeptide comprising at least about 80%, 85%, 90%, 95%, 96%, 97%, 98%, or 99% identity to any one of SEQ ID NOS: 2, 11, 13, 19, or 21. A polynucleotide-based vaccine, or polynucleotide vaccine, of the present invention is capable of eliciting, without more, an immune response in an animal against a Chlamydia species, e.g., C. trachomatis or C. pneumoniae, when administered to that animal.

Polynucleotide-based vaccines compositions of the invention include nucleic acid-mediated modalities. DNA or RNA encoding one or more of the polypeptides of the invention can also be administered to a patient. This approach is described, for instance, in Wolff et. al., Science 247: 1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

In some embodiments, the polynucleotide-based vaccines are prepared and administered in such a manner that the encoded gene products are optimally expressed in the particular animal to which the composition is administered. As a result, these compositions and methods are useful in stimulating an immune response against Chlamydia infection as the coding sequence encodes a polypeptide which stimulates the immune system to respond to Chlamydia infection. Also included in the invention are expression systems, delivery systems, and codon-optimized Chlamydia coding sequences, e.g., viral vectors. Vaccinia vectors (e.g., Modified Vaccinia Ankara (MVA)) and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the polypeptides of the invention, e.g., adeno and adeno-associated virus vectors, retroviral vectors, Salmonella typhi vectors (see, for instance, WO 00/014240, WO 00/068261, and WO 02/072845, each of which is herein incorporated by reference in its entirety), detoxified anthrax toxin vectors, and the like, will be apparent to those skilled in the art from the description herein.

In certain embodiments, the polynucleotides are complexed in a liposome preparation (Felgner et al., Proc. Natl. Acad. Sci. USA 84:74137416 (1987); Malone et al., Proc. Natl. Acad. Sci. USA 86:60776081 (1989)). Furthermore, polynucleotide-vaccine compositions of the present invention may include one or more transfection facilitating compounds that facilitate delivery of polynucleotides to the interior of a cell, and/or to a desired location within a cell.

In other embodiments, the polynucleotide itself may function as an adjuvant as is the case when the polynucleotides of the invention are derived, in whole or in part, from bacterial DNA. Bacterial DNA containing motifs of unmethylated CpG-dinucleotides (CpG-DNA) triggers innate immune cells in animals through a pattern recognition receptor (including toll receptors such as TLR 9) and thus possesses potent immunostimulatory effects on macrophages, dendritic cells and B-lymphocytes. See, e.g., Wagner, H., Curr. Opin. Microbiol. 5:62-69 (2002); Jung, J. et al., J. Immunol. 169: 2368-73 (2002); see also Klinman, D. M. et al., Proc. Natl. Acad. Sci. U.S.A. 93:2879-83 (1996). Methods of using unmethylated CpG-dinucleotides as adjuvants are described in, for example, U.S. Pat. Nos. 6,207,646, 6,406,705, and 6,429,199, the disclosures of which are herein incorporated by reference.

Compositions comprising polynucleotides of the present invention may include various salts, excipients, delivery vehicles and/or auxiliary agents as are disclosed, e.g., in U.S. Pat. No. 6,875,748, which is incorporated herein by reference in its entirety.

### Live Carrier Vaccines

In some embodiments, the present invention includes live carrier vaccines. For instance, an avirulent host cell can be used as a carrier to deliver a nucleic acid and/or polypeptide of the invention to a subject. The host cell carrier can be prokaryotic, eukaryotic or viral. In one embodiment, the host cell carrier has been modified to make it attenuated or avirulent.

The invention includes, for instance, a vaccine comprising an attenuated gram-negative pathogen as a carrier for a nucleic acid or polypeptide of the invention. In one embodiment, the gram negative pathogen is a Salmonella enterica serovar, for instance, S. typhi or S. typhimurium. The attenuated Salmonella vaccine carrier can have at least one attenuating mutation in the Salmonella Pathogenicity Island 2 (SPI2) region as described in U.S. Pat. Nos. 6,342,215 and 6,936,425, both of which are herein incorporated by reference in their entireties. In another embodiment, the attenuated Salmonella vaccine carrier comprises attenuating mutations in a second gene associated with virulence (e.g., aro or sod). For instance, the invention includes an attenuated Salmonella enterica host cell with attenuating mutations or inactivating mutations in an aro gene (e.g., aroC gene) and a SPI2 gene (e.g., ssaV gene) as described in U.S. Pat. No. 6,756,042, which is herein incorporated by reference in its entirety.

A live, attenuated S. enterica vaccine capable of expressing a polypeptide of the invention can be prepared using cloning methods known in the art. For instance, a nucleic acid encoding a soluble polypeptide with at least about 70%, 75%, 80%, 85%, 90%, 95%, 96%, 97%, 98% or 99% identity to any one of SEQ ID NOS: 2, 11, 13, 15, 19 and 21 or amino acids 42-743 of SEQ ID NO: 29 or amino acids 64-765 of SEQ ID NO: 32 can be expressed from a plasmid or can be incorporated into the host cell's genome. In one embodiment, the gene expression cassette is inserted in a mutated gene of the Salmonella sp., for instance, in the aroC or ssaV genes. In one embodiment, the construct is a deletion/insertion construct (i.e., at least one Salmonella gene contains a deletion mutation and the gene expression cassette comprising the nucleic acid sequence encoding the Chlamydia polypeptide is inserted in the deletion sites). In another embodiment, the nucleic acid encoding the Chlamydia polypeptide is under the control of a Salmonella enterica promoter, for instance a ssaG promoter. In one embodiment, the organism is an attenuated Salmonella typhi or typhimurium with deletion mutations in the ssaV and aroC genes, and a gene cassette comprising a Chlamydia nucleic acid sequence under the control of a ssaG promoter is inserted in the aroC and/or ssaV deletion sites. See, for instance, WO 00/14240 and WO 02/072845, each of which is herein incorporated by reference in its entirety.

In another embodiment, a live, avirulent viral vaccine carrier can be used to deliver a nucleic acid and/or polypeptide of the invention in a subject. Such viral vaccine carriers include, but are not limited to Modified Vaccinia Virus (e.g., MVA) and Moloney Murine Leukemia Virus. For instance, using cloning methods generally available in the art, MVA can be used as a carrier for a nucleic acid encoding a soluble polypeptide with at least about 70%, 75%, 80%, 85%, 90%, 95%, 96%, 97%, 98% or 99% identity to any one of SEQ ID NOS: 2, 11, 13, 15, 19 and 21 or amino acids 42-743 of SEQ ID NO: 29 or amino acids 64-765 of SEQ ID NO: 32.

The invention includes a method of eliciting an immunogenic response in a subject by administering to the subject a live vaccine carrier comprising a nucleic acid of the invention, for instance, a nucleic acid encoding a soluble polypeptide with at least about 70%, 75%, 80%, 85%, 90%, 95%, 96%, 97%, 98% or 99% identity to any one of SEQ ID NOS: 2, 11, 13, 15, 19 and 21 or amino acids 42-743 of SEQ ID NO: 29 or amino acids 64-765 of SEQ ID NO: 32. In one embodiment, the immunogenic response is a protective immune response. The immune response can be a humoral or cellular immune response.

In one embodiment, a live vaccine carrier (e.g., Salmonella enterica or MVA) comprising a nucleic acid of the invention for instance, a nucleic acid encoding a soluble polypeptide with at least about 70%, 75%, 80%, 85%, 90%, 95%, 96%, 97%, 98% or 99% identity to any one of SEQ ID NOS: 2, 11, 13, 15, 19 and 21, can be administered to a subject to prevent or treat a C. trachomatis infection or a condition associated with a Chlamydia trachomatis infection (e.g., prostatitis, urethritis, epididymitis, cervicitis, pelvic inflammatory disease, pelvic pain, newborn eye infection, newborn lung infection, infertility, proctitis, reactive arthritis and trachoma). In another embodiment, the invention includes treating or preventing a C. pneumoniae infection or condition associated with C. pneumoniae infection (e.g., pneumonia, acute respiratory disease, atherosclerosis, coronary artery disease, myocardial infarction, carotid artery disease, cerebrovascular disease, coronary heart disease, carotid artery stenosis, aortic aneurysm, claudication and stroke) by administering to a subject a live vaccine carrier (e.g., Salmonella enterica or MVA) comprising a nucleic acid of the invention for instance, a nucleic acid encoding a soluble polypeptide with at least about 70%, 75%, 80%, 85%, 90%, 95%, 96%, 97%, 98% or 99% identity to amino acids 42-743 of SEQ ID NO: 29 or amino acids 64-765 of SEQ ID NO: 32. The methods of the invention include administering the live vaccine carrier composition to a subject at an effective dose (e.g., the dose necessary to elicit an immune response and/or to ameliorate a condition associated with a Chlamydial infection).

The invention includes a composition comprising a live vaccine (e.g., Salmonella enterica or MVA) carrier comprising a nucleic acid of the invention (e.g., a nucleic acid encoding a soluble polypeptide with at least about 70%, 75%, 80%, 85%, 90%, 95%, 96%, 97%, 98% or 99% identity to any one of SEQ ID NOS: 2, 11, 13, 15, 19 and 21 or amino acids 42-743 of SEQ ID NO: 29 or amino acids 64-765 of SEQ ID NO: 32). In one embodiment, the composition is a vaccine composition. In another embodiment, the composition is a pharmaceutical composition. In another embodiment, the composition is an immunogenic composition.

The live vaccine carrier composition of the invention can further comprise, for instance, a pharmaceutically acceptable carrier, diluent, excipient and/or adjuvant as provided herein. The live vaccine carrier composition of the invention can be administered by methods known in the art (e.g., injection, for instance, by i.v. or i.m., oral, transmucosal).

### Methods of Treatment/Prevention and Regimens

Also provided is a method to treat or prevent a Chlamydia infection in an animal comprising: administering to the animal in need thereof a composition containing any one of the polypeptides or polynucleotides of the present invention. In some embodiments, the invention is directed to a method of inducing an immune response against Chlamydia in a host animal comprising administering an effective amount a composition containing any one of the polypeptides or polynucleotides of the present invention.

In some embodiments, an animal can be treated with the compositions, polypeptides or polynucleotides prophylactically, e.g., as a prophylactic vaccine, to establish or enhance immunity to one or more Chlamydia species, e.g., Chlamydia trachomatis, in a healthy animal prior to exposure to Chlamydia or contraction of a Chlamydia symptom, thus preventing the disease or reducing the severity of disease symptoms. One or more compositions, polypeptides or polynucleotides of the present invention may also be used to treat an animal already exposed to Chlamydia, or already suffering from Chlamydia-related symptom to further stimulate the immune system of the animal, thus reducing or eliminating the symptoms associated with that exposure. As defined herein, “treatment of an animal” refers to the use of one or more compositions, polypeptides or polynucleotides of the present invention to prevent, cure, retard, or reduce the severity of Chlamydia symptoms in an animal, and/or result in no worsening of Chlamydia symptoms over a specified period of time. It is not required that any composition, polypeptides or polynucleotides of the present invention provides total protection against Chlamydia infection or totally cure or eliminate all Chlamydia symptoms. As used herein, “an animal in need of therapeutic and/or preventative immunity” refers to an animal which it is desirable to treat, i.e., to prevent, cure, retard, or reduce the severity of Chlamydia symptoms, and/or result in no worsening of Chlamydia symptoms over a specified period of time.

In some embodiments, an antibody specifically reactive with a Chlamydia organism is isolated from the serum of the host animal which has been administered a polypeptide or polynucleotide of the present invention. In some embodiments, the invention is directed to a method of providing passive immunity comprising administering the antibody specifically reactive with a Chlamydia organism (which was isolated from the serum of a host animal) to an animal in need thereof.

Treatment with pharmaceutical compositions comprising the immunogenic compositions, polypeptides or polynucleotides of the present inventions can occur separately or in conjunction with other treatments, as appropriate.

In therapeutic applications, compositions, polypeptides or polynucleotides are administered to a patient in an amount sufficient to elicit an effective CTL response to the Chlamydia-derived polypeptide to cure or at least partially arrest symptoms and/or complications. An amount adequate to accomplish this is defined as “therapeutically effective dose” or “unit dose.” Amounts effective for this use will depend on, e.g., the polypeptide or polynucleotide composition, the manner of administration, the stage and severity of the disease being treated, the weight and general state of health of the patient, and the judgment of the prescribing physician, but generally range for the initial immunization for polypeptide vaccines is (that is for therapeutic or prophylactic administration) from about 1.0 μg to about 5000 μg of polypeptide, in some embodiments about 30 μg to about 200 μg or about 10 μg to about 30 μg, for a 70 kg patient, followed by boosting dosages of from about 1.0 μg to about 1000 μg, in some embodiments 10 μg to about 30 μg, of polypeptide pursuant to a boosting regimen over weeks to months depending upon the patient's response and condition by measuring specific CTL activity in the patient's blood. In alternative embodiments, generally for humans the dose range for the initial immunization (that is for therapeutic or prophylactic administration) is from about 1.0 μg to about 20,000 μg of polypeptide for a 70 kg patient, in some embodiments 2 μg-, 5 μg-, 10 μg-, 15 μg-, 20 μg-, 25 μg-, 30 μg-, 40 μg-, or 50 μg-2000 μg, followed by boosting dosages in the same dose range pursuant to a boosting regimen over weeks to months depending upon the patient's response and condition by measuring specific CTL (cytotoxic T lymphocytes) activity in the patient's blood. In a specific, non-limiting embodiment of the invention, approximately 0.01 μg to 2000 μg, or in some embodiments 2 μg to 200 μg or 10 μg to 30 μg, of a polypeptide or polynucleotide of the present invention, or its fragment, derivative variant, or analog is administered to a host.

In embodiments where DNA vaccine administration is used, the amount of polynucleotide in the initial immunization (that is for therapeutic or prophylactic administration) depends upon a number of factors including, for example, the antigen being expressed, the expression vector being used, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian. In some embodiments, doses for nucleic acids encoding polypeptides range from about 10 ng to 1 g, 100 ng to 100 mg, 1 μg to 10 mg, or 30 to 300 μg DNA or RNA per patient.

In non-limiting, embodiments of the invention, an effective amount of a composition of the invention produces an elevation of antibody titer to at least three times the antibody titer prior to administration.

It must be kept in mind that the polypeptides and compositions of the present invention may generally be employed in serious disease states, that is, life-threatening or potentially life threatening situations. In such cases, in view of the minimization of extraneous substances and the relative nontoxic nature of the polypeptides, it is possible and may be felt desirable by the treating physician to administer substantial excesses of these polypeptide compositions.

For therapeutic use, administration should begin at the first sign of Chlamydia infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

In certain embodiments, one or more compositions of the present invention are delivered to an animal by methods described herein, thereby achieving an effective immune response, and/or an effective therapeutic or preventative immune response. Any mode of administration can be used so long as the mode results in the delivery and/or expression of the desired polypeptide in the desired tissue, in an amount sufficient to generate an immune response to Chlamydia, e.g., C. trachomatis, and/or to generate a prophylactically or therapeutically effective immune response to Chlamydia, e.g., C. trachomatis, in an animal in need of such response. According to the disclosed methods, compositions of the present invention can be administered by intradural injection, subcutaneous injection, intravenous injection, oral administration, or pulmonary administration or intramuscular (i.m.) administration. Other suitable routes of administration include, but not limited to intratracheal, transdermal, intraocular, intranasal, inhalation, intracavity, intraductal (e.g., into the pancreas) and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), inraatrial (i.e., into the heart atrium) and sub arachnoid (i.e., into the sub arachnoid spaces of the brain) administration.

Upon immunization with a polypeptide or polynucleotide composition in accordance with the invention, the immune system of the host responds to the vaccine by producing large amounts of HTLs (helper T lymphocytes) and/or CTLs (cytotoxic T lymphocytes) specific for the desired antigen. Consequently, the host becomes at least partially immune to later infection, or at least partially resistant to developing an ongoing chronic infection.

In some embodiments, polypeptides or polynucleotides of the present invention stimulate a cell-mediated immune response sufficient for protection of an animal against Chlamydia infection. In other embodiments, polypeptides or polynucleotides of the present invention stimulate both a humoral and a cell-mediated response, the combination of which is sufficient for protection of an animal against Chlamydia protection.

In certain embodiments, components that induce T cell responses are combined with components that induce antibody responses to the target antigen of interest. Thus, in certain embodiments of the invention, vaccine compositions of the invention are combined with polypeptides or polynucleotides which induce or facilitate neutralizing antibody responses to the target antigen of interest. One embodiment of such a composition comprises a class I epitope in accordance with the invention, along with a PADRE® (Epimmune, San Diego, Calif.) molecule (described, for example, in U.S. Pat. No. 5,736,142).

The polynucleotides of the present invention, or vectors containing the polynucleotides of the present invention, can be incorporated into the cells of the animal in vivo, and an antigenic amount of the C. trachomatis-derived polypeptide, or fragment, variant, or derivative thereof, is produced in vivo. Upon administration of the composition according to this method, the C. trachomatis-derived polypeptide is expressed in the animal in an amount sufficient to elicit an immune response. Such an immune response might be used, for example, to generate antibodies to C. trachomatis for use in diagnostic assays or as laboratory reagents.

The present invention further provides a method for generating, enhancing, or modulating a protective and/or therapeutic immune response to C. trachomatis in an animal, comprising administering to the animal in need of therapeutic and/or preventative immunity one or more of the compositions described herein. In some embodiments, the composition includes an isolated polynucleotide comprising a codon-optimized coding region encoding a polypeptide of the present invention, optimized for expression in a given host organism, e.g., a human, or a nucleic acid of such a coding region encoding a fragment, variant, or derivative thereof. The polynucleotides are incorporated into the cells of the animal in vivo, and an immunologically effective amount of the C. trachomatis polypeptide, or fragment or variant is produced in vivo. Upon administration of the composition according to this method, the C. trachomatis-derived polypeptide is expressed in the animal in a therapeutically or prophylactically effective amount.

The compositions of the present invention can be administered to an animal at any time during the lifecycle of the animal to which it is being administered. For example, the composition can be given shortly after birth. In humans, administration of the composition of the present invention can occur while other vaccines are being administered, e.g., at birth, 2 months, 4 months, 6 months, 9 months, at 1 year, at 5 years, or at the onset of puberty. In some embodiments, administration of the composition of the present invention can occur when the human become sexually active.

Furthermore, the compositions of the invention can be used in any desired immunization or administration regimen; e.g., in a single administration or alternatively as part of periodic vaccinations such as annual vaccinations, or as in a prime-boost regime wherein the polypeptide or polynucleotide of the present invention is administered either before or after the administration of the same or of a different polypeptide or polynucleotide.

Recent studies have indicated that a prime-boost protocol is often a suitable method of administering vaccines. In a prime-boost protocol, one or more compositions of the present invention can be utilized in a “prime boost” regimen. An example of a “prime boost” regimen may be found in Yang, Z. et al. J. Virol. 77:799-803 (2002), which is incorporated herein by reference in its entirety. In a non-limiting example, one or more polynucleotide vaccine compositions of the present invention are delivered to an animal, thereby priming the immune response of the animal to a Chlamydia polypeptide of the invention, and then a second immunogenic composition is utilized as a boost vaccination. One or more compositions of the present invention are used to prime immunity, and then a second immunogenic composition, e.g., a recombinant viral vaccine or vaccines, a different polynucleotide vaccine, or one or more purified subunit of the Chlamydia polypeptides or fragments, variants or derivatives thereof is used to boost the anti-Chlamydia immune response.

In another non-limiting example, a priming composition and a boosting composition are combined in a single composition or single formulation. For example, a single composition may comprise an isolated Chlamydia polypeptide or a fragment, variant, or derivative thereof as the priming component and a polynucleotide encoding a Chlamydia polypeptide as the boosting component. In this embodiment, the compositions may be contained in a single vial where the priming component and boosting component are mixed together. In general, because the peak levels of expression of polypeptide from the polynucleotide does not occur until later (e.g., 7-10 days) after administration, the polynucleotide component may provide a boost to the isolated polypeptide component. Compositions comprising both a priming component and a boosting component are referred to herein as “combinatorial vaccine compositions” or “single formulation heterologous prime-boost vaccine compositions.” In addition, the priming composition may be administered before the boosting composition, or even after the boosting composition, if the boosting composition is expected to take longer to act.

In another embodiment, the priming composition may be administered simultaneously with the boosting composition, but in separate formulations where the priming component and the boosting component are separated.

### Kits

The polypeptide or polynucleotide vaccine compositions of this invention can be provided in kit form together with a means for administering the polypeptide, polynucleotide, or composition of the present invention. In some embodiments, the kit can further comprise instructions for vaccine administration.

Typically the kit would include desired composition(s) of the invention in a container, e.g., in unit dosage form and instructions for administration. Means for administering the composition of the present invention can include, for example, a sterile syringe, an aerosol applicator (e.g., an inhaler or any other means of nasal or pulmonary administration), a gel, a cream, a transdermal patch, transmucosal patch (or any other means of buccal or sublingual administration), or an oral tablet. In some embodiments, the kit of the present invention contains two or more means for administering the polypeptides, polynucleotides, vectors, or compositions of the present inventions, e.g., two or more syringes.

In some embodiments, the kit may comprise more than one container comprising the polypeptide, polynucleotide, or composition of the present invention. For example, in some embodiments the kit may comprise a container containing a priming component of the present invention, and a separate container comprising the boosting component of the present invention.

Optionally associated with such container(s) can be a notice or printed instructions. For example, such printed instructions can be in a form prescribed by a governmental agency regulating the manufacture, use or sale of pharmaceuticals or biological products, which notice reflects approval by the agency of the manufacture, use or sale for human administration of the present invention. “Printed instructions” can be, for example, one of a book, booklet, brochure or leaflet.

The kit can also include a storage unit for storing the components (e.g., means of administering, containers comprising the polypeptides, polynucleotides, or compositions of the present inventions, printed instructions, etc.) of the kit. The storage unit can be, for example, a bag, box, envelope or any other container that would be suitable for use in the present invention. Preferably, the storage unit is large enough to accommodate each component that may be necessary for administering the methods of the present invention.

The present invention can also include a method of delivering a polypeptide, polynucleotide, or composition of the present invention to an animal such as a human in need thereof, the method comprising (a) registering in a computer readable medium the identity of an administrator (e.g., a physician, physician assistant, nurse practitioner, pharmacist, veterinarian) permitted to administer the polypeptide, polynucleotide, vector, or composition of the present invention; (b) providing the human with counseling information concerning the risks attendant the polypeptide, polynucleotide, vector, or composition of the present invention; (c) obtaining informed consent from the human to receive the polypeptide, polynucleotide, vector, or composition of the present invention despite the attendant risks; and (e) permitting the human access to the polypeptide, polynucleotide, vector, or composition of the present invention.

### Immunoassays

The present invention also provides assays for detecting or measuring an immune response to polypeptides of the present invention. In some embodiments, the immune response of an organism can be determined by comparing the sera from an organism that is unvaccinated, or that has not been exposed to an antigen originating from Chlamydia (preimmune sera), to the sera from an organism that has been vaccinated, or that has been exposed to an antigen originating from Chlamydia (immune sera). As used herein, “a detectable immune response” refers to an immunogenic response to the polynucleotides and polypeptides of the present invention, which can be measured or observed by standard protocols.

Standard protocols for detecting an immune response include, but are not limited to, immunoblot analysis (western), fluorescence-activated cell sorting (FACS), radioimmunoassays, ELISA (enzyme linked immunosorbent assay), “sandwich” immunoassays, immunoprecipitation analysis, cytolytic T-cell response, ELISPOT, and chromium release assay. An immune response may also be “detected” through challenge of immunized animals with a virulent Chlamydia species, either before or after vaccination. Standard chromium release assays are used to measure specific cytotoxic T lymphocyte (CTL) activity against the Chlamydia antigens. More sensitive techniques such as the ELISPOT assay, intracellular cytokine staining, and tetramer staining have become available in the art to determine lymphocyte antigen responsiveness. It is estimated that these newer methods are 10- to 100-fold more sensitive than the common CTL and HTL assays (Murali-Krishna et al., Immunity, 8:177-87 (1998)), because the traditional methods measure only the subset of T cells that can proliferate in vitro, and may, in fact, be representative of only a fraction of the memory T cell compartment (Ogg G. S., McMichael A. J., Curr Opin Immunol, 10:393-6 (1998)).

Western blot analysis generally comprises preparing protein samples, e.g., polypeptides of the present invention, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody, e.g., serum from vaccinated individuals, preimmune sera and control positive antibodies, diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing polypeptide of the invention, coating the well of a 96 well microtiter plate with a polypeptide of the invention, adding test antibodies (e.g., from immune sera in serial dilutions) and control antibodies (e.g., from preimmune sera) to the microtiter plate as described above, and incubating for a period of time. Then a second antibody (which recognizes the antibody of interest) conjugated to a detectable compound may be added to the well, wherein the second antibody is conjugated to a detectable compound such as an enzymatic substrate. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical -Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4^{th} ed. Ed. Richard A. Goldsby, Thomas J. Kindt and Barbara A. Osborne, H. Freemand & Co. (2000); Roitt, I., Brostoff, J. and Male D., Immunology, 6^{th} ed. London: Mosby (2001); Abbas A., Abul, A. and Lichtman, A., Cellular and Molecular Immunology, Ed. 5, Elsevier Health Sciences Division (2005); Kontermann and Dubel, Antibody Engineering, Springer Verlan (2001); Sambrook and Russell, Molecular Cloning: A Laboratory Manual, Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer, Cold Spring Harbor Press (2003).

### EXAMPLES

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments in accordance with the invention.

### Example 1

### Construction of pET15b-CT84 Plasmid

The CT84 gene fragment (SEQ ID NO:1) was PCR amplified then inserted into a pET plasmid, resulting in a pET15b-CT84 plasmid which encodes a His-tagged CT84 polypeptide. Specifically, pET15b-CT84 was created by inserting the CT84 gene into pET15b-Spe, a vector derived from pET15b (Novagen). The pET15b-Spe vector is the same as pET15b, except that an extra Spe1 restriction site was added in-frame immediately upstream of BamH1 using common molecular biology techniques. The pET15b-Spe vector has a His-tag upstream of the multiple cloning sites. The CT84 gene, which comprises amino acid 29 to 784 of CT110, was PCR-amplified from the purified CT110 plasmid DNA using the following primers:

(SEQ ID NO: 24)
5′- GGGAATTCCCATATGGAAATCATGGTTCCTCAAGGAATTTAC -3′
and
(SEQ ID NO:25)
5′- CGACTAGTTTATTAGGTAAATGCTAGACCAAACATCG -3′

The PCR product was restricted with Nde1 and Spe1 and ligated into pET15b-Spe vector that had been restricted with Nde1 and Spe1, resulting in a pET15b-CT84 plasmid in which the plasmid encoded a His-tagged CT84 polypeptide. The pET15b-CT84 plasmid was then transformed into E. coli strain BL21(DE3) or BL21(DE3)pLysS. Transcription of the CT84 gene in the pET15b-CT84 plasmid was controlled by the T7 promoter. The His-tagged CT84 protein was expressed by inducing the BL21(DE3) host cells with IPTG.

### Example 2

### Construction of pET15b-CT57 Plasmid

The CT57 gene fragment (SEQ ID NO:8) was PCR amplified then inserted into pET15b plasmid, resulting in a pET15b-CT57 plasmid which encodes a His-tagged CT57 polypeptide. Specifically, the CT57 gene was cloned into pET15b-Spe. The CT57 gene was PCR-amplified from the purified CT110 plasmid DNA using the following primers:

(SEQ ID NO: 26)
5′- GGGAATTCCCATATGGCTCAAGCTGATGGGGGAGCTTGTC -3′
and
(SEQ ID NO: 27)
5′- CGACTAGTTTATTAATGCGCAGATCGTATATCTAAAATGG -3′.

The PCR product was restricted with Nde1 and Spe1 and ligated into pET15b-Spe vector that had been restricted with Nde1 and Spe1, resulting in a pET15b-CT57 plasmid in which the plasmid encoded a His-tagged CT57 polypeptide. The pET15b-CT57 plasmid was then transformed into E. coli strain BL21(DE3) or BL21(DE3)pLysS. Transcription of the CT57 gene in the pET15b-CT57 plasmid was controlled by the T7 promoter. The His-tagged CT57 protein was expressed by inducing the BL21(DE3) host cells with IPTG.

### Example 3

### Construction of pET15b-CT40 Plasmid

The CT40 gene fragment (SEQ ID NO:6) was PCR amplified then inserted into pET15b plasmid, resulting in a pET15b-CT40 plasmid which encodes a His-tagged CT40 polypeptide. Specifically, the CT40 gene was cloned into pET15b-Spe. The CT40 gene was PCR amplified from the purified CT110 plasmid DNA using the following primers:

(SEQ ID NO: 28)
5′- GGGAATTCCCATATGATTTTCGATGGGAATATTAAAAGAACAG
CC -3′
and
(SEQ ID NO: 17)
5′- CGACTAGTTTATTAGGTAAATGCTAGACCAAACATCG -3′.

The PCR product was restricted with Nde1 and Spe1 and ligated into pET15b-Spe vector that had been restricted with Nde1 and Spe1, resulting in a pET15b-CT40 plasmid in which the plasmid encoded a His-tagged CT40 polypeptide. The pET15b-CT40 plasmid was then transformed into E. coli strain BL21(DE3) or BL21(DE3)pLysS. Transcription of the CT40 gene in the pET15b-CT40 plasmid was controlled by the T7 promoter. The His-tagged CT40 protein was expressed by inducing the BL21(DE3) host cells with IPTG.

### Example 4

### Expression of pET 15b-Based Plasmids

The pET15b-CT84, pET15b-CT57, and pET15b-CT40 plasmids were then transformed into E. coli strain BL21(DE3) or BL21(DE3)pLysS. Transcription of the CT84, CT57, and CT40 genes in the pET15b-based plasmids was controlled by the T7 promoter.

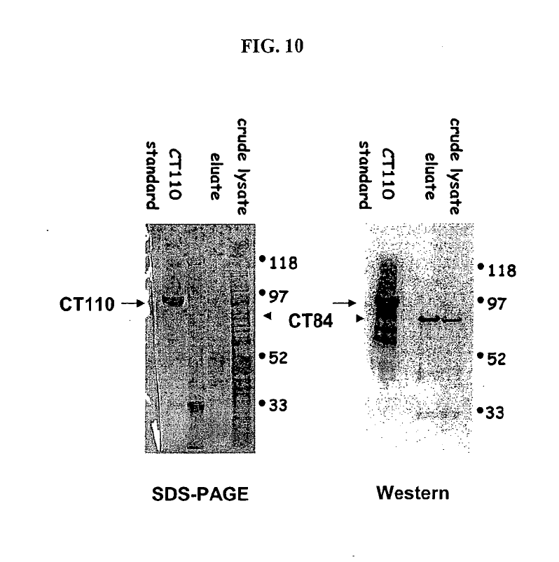

BL21(DE3) bacterial cells containing one of the CT110 truncation expression plasmids were grown in 10 mL of Miller (Luria-Bertani), LB broth. When the O.D.₆₀₀ reached 0.8, expression of the CT84, CT40, or CT57 gene was induced by addition of IPTG at 37° C. for 2 hours with shaking. Previous experiments with CT84 demonstrated that induction over 2 hours did not further increase protein expression. After induction, the cell culture was centrifuged to collect the cell pellet. The pellets were then resuspended in 20 mM Tris-HCl, 200 mM NaCl, and 8 M urea, pH 8.0. Cell lysates were prepared by sonication. Supernatants were collected and incubated with 1 mL of nickel sepharose beads for a few hours at 4° C. The beads were spun down, washed, and the CT110 truncated proteins was eluted with 20 mM Tris-HCl, 200 mM NaCl, 8 M urea and 200 mM imidazole, pH 8.0. The total lysate, flow-through and eluate were run on a 4-12% reducing SDS-PAGE gel, and visualized using Western blot analysis by anti-CT110 and anti-His antibodies (Novagen) (FIG. 10). The anti-CT110 antibody is a polyclonal antibody raised against rabbit using the full-length CT110 antigens prepared according to U.S. Pat. No. 6,642,023.

### Example 5

### Isolation of CT84, CT40, and CT57

The BL21(DE3) bacterial cells comprising one of pET15b-CT84, pET15b-CT40, pET15b-CT57 plasmids were grown in 2 L of Miller (Luria-Bertani), LB broth, in flasks. When the O.D.₆₀₀ reached 0.8, expression of the CT84, CT40 or CT57 gene was induced by addition of IPTG at 37° C. for 2 hours with shaking. At the end of induction, the cell culture was centrifuged to collect the cell pellet. The cell pellet was washed once with 20 mM Tris-HCl, pH 8.0 and resuspended in 20 mM Tris-HCl, 2% sodium deoxycholate, pH8.0. The bacterial cells were lysed using sonication (5-s burst at power 5.0 for 5-6 times), then the inclusion bodies were washed four times—twice with the same buffer, once with 20 mM Tris-HCl and once with water—to remove the deoxycholate. The inclusion bodies were then solubilized in 6M urea overnight at 4° C. with rocking. The solubilized crude cell lysate containing the CT84, CT40 or CT57 proteins was clarified using a 0.4 μm filter.

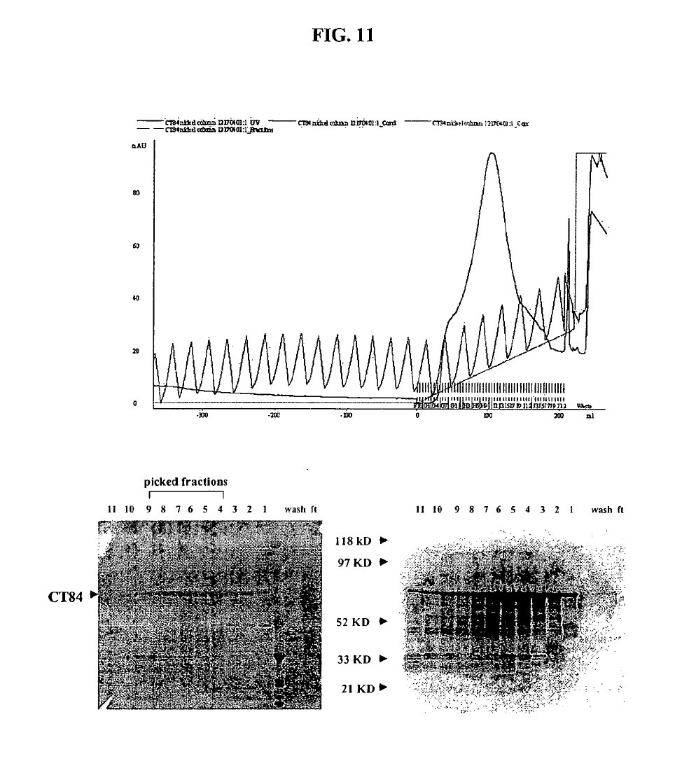

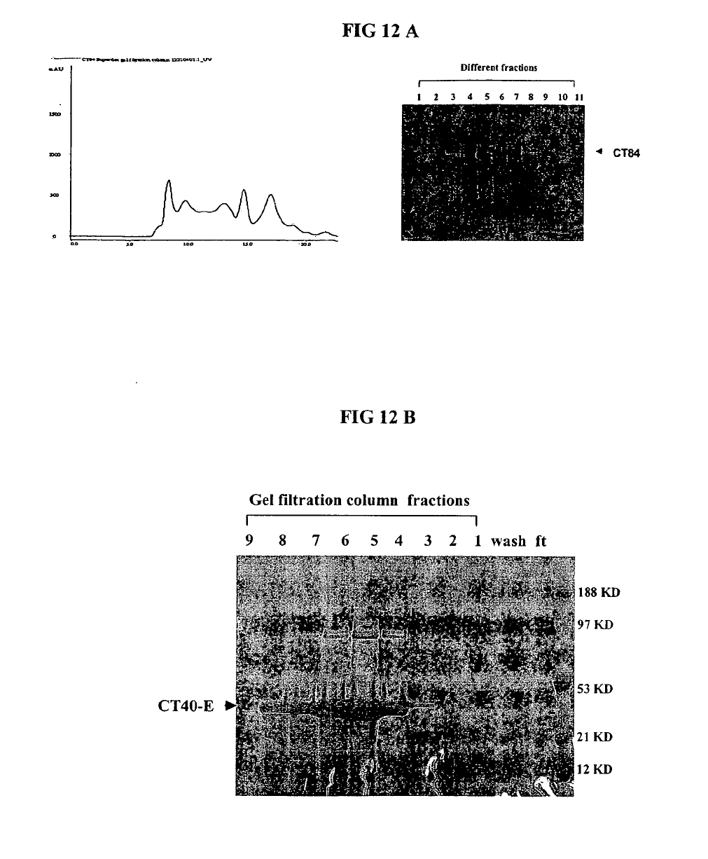

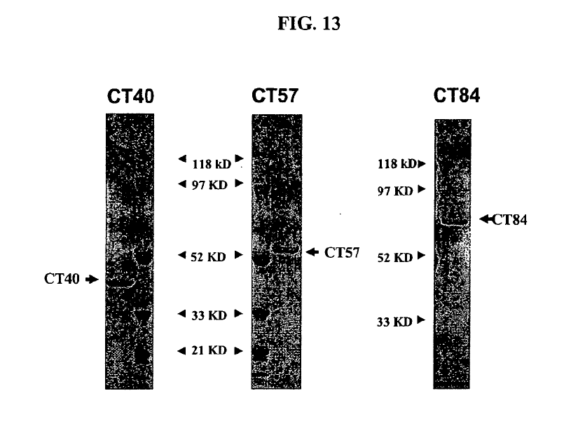

The clarified cell lysate was loaded onto a pre-packed nickel affinity column (GE) and protein purification was performed using an AKTA FPLC system (GE). After loading the sample, the column was washed with buffer A (20 mM sodium phosphate, 500 mM NaCl, 6 M urea, 10 mM imidazole, pH 8.0). After wash, the target protein was eluted using a gradient of 0 to 30% of buffer B (20 mM sodium phosphate, 500 mM NaCl, 6 M urea, 500 mM imidazole, pH 8.0). The eluted fractions were run on a 4-12% SDS-PAGE gel and desired fractions were selected by visualizing the target protein using western blotting (FIG. 11). The eluted protein was again loaded on a pre-packed Superdex 200 gel filtration column (GE). Protein was eluted using 20 mM sodium phosphate, 500 mM NaCl, and 6 M urea, pH 7.6. Again, the eluted fractions were run on a 4-12% SDS-PAGE gel and the fractions that contained the purified CT84, CT40 or CT57 were visualized and selected using western blotting (FIGS. 12A and 12B). The selected fractions were combined. Step-down dialysis was performed sequentially to exchange the buffer first from one that contained 6 M urea to one that contained 4 M urea, then to 2 M urea, and finally, to PBS, or other isotonic saline buffer suitable for injection into animals. The purity of CT84, CT40 and CT57 was determined by densitometry, and was determined to be between 87% and 96% (FIG. 13). A total of 3 mg of protein was purified from 2 L of cell culture. Purified protein was concentrated by Centricon (Millipore) to 1 mg/mL and stored at −80° C. The full-length CT110 protein solution appears to be a suspension in PBS or water with visually observable particulates. The CT84 protein solution in PBS or water is clear and visually free of precipitates, even after 3 months of storage at −80° C.

### Example 6

### Construction of pLex-CT84 Plasmid

The CT84 gene fragment was PCR amplified using the following primers:

CT84(OPT)-NdeI-for
(SEQ ID NO: 4)
GGGAATTCCATATGGAAATTATGGTTCCGCAGGGTATC
CT84(OPT)-XbaI-rev
(SEQ ID NO: 5)
CTAGTCTAGATTAGGTGAACGCCAGGCCGAACATG

The CT84 PCR product was restricted with NdeI and XbaI and ligated into pLEX vector that had been restricted with NdeI and XbaI, resulting in a pLex-CT84 plasmid which encodes a CT84 polypeptide.

The pLEX-Ct84 plasmid was then transformed into E. coli strain GI724. Transcription of CT84 gene in the pLEX-CT84 plasmid was controlled by the PL promoter and the cI repressor. The GI724 bacterial cells comprising the pLEX-CT84 plasmid was grown in a media containing: 1×M9 salts, 2% casamino acids, 0.5% glucose, 1 mM MgCl₂, 50 μg/ml kanamycin, and 100 μg/ml tryptophan.

CT84 was expressed by inducing the GI724 bacterial cells comprising the pLEX-CT84 plasmid at 30° C. for 16 hours. CT84 protein purified from the pLEX expression system was visually soluble in 50 mM Tris-HCl, Tween 80 (0.05 to 0.2%), pH 8.0. Moreover, spectrofluorometry was used to determine the protein solubility and folding by monitoring emission spectrum around 330 to 335 nm. Proteins in the range indicate that CT84 was folded at least partially. The aggregated protein emits fluorescence above 340 nm. The CT84 solution was centrifuged in a microcentrifuge at 14,000 rpm for 30 min. The precipitated and soluble parts were loaded on a Coomassie gel and no CT84 was shown on precipitated lane.

### Example 7

### Construction of pET-43.1 EK/LIC and pRSF2 EK/LIC plasmids

PmpD-133, PmpH-78, PmpI-63, OmcB-1, and OmpH were also cloned into pET-43.1 EK/LIC (Novagen) and pRSF2 (Novagen) plasmids. pET-43.1 EK/LIC plasmids provide both a His-tagged and an Nus-fusion protein. To clone into the pET-43.1 Ek/LIC, primers were made and specific 5′ and 3′ LIC extensions, i.e., 5′-GACGACGACAAG (SEQ ID NO:22), and 5′-GAGGAGAAGCCCGGT (SEQ ID NO:23) were put in front of the target gene sequences. After PCR amplification using these primers, the PCR inserts were treated with T4 DNA Polymerases and dATP, annealed to pET-43.1 EK/LIC plasmids, and transformed into appropriate competent E. coli host cells.

### Example 8

### Construction of pET15b Plasmids

PmpD-133, PmpH-78, PmpI-63, OmcB-1, and OmpH were also cloned into pET15b (Novagen). PCR inserts were made by amplifying the Chlamydia DNA templates using appropriate primers. Both the vector and PCR inserts were digested with NdeI and BamHI, gel cleaned, annealed and transformed into appropriate competent E. coli host cells.

### Example 9

### Expression and Purification of OmcB-1

OmcB-1 was expressed from a pRRSF2 vector in a host cell in shaker flasks at 37° C. and induced by IPTG for 3 hours. The host cells were then harvested. Cell paste (5.8 g) was resuspened in 45 ml of Resuspension Buffer (10 mM imidazole-HCl, 20 mM sodium phosphate, 10 mM EDTA, 300 mM NaCl, pH 7.4) plus 100 ug/ml lysozyme, and incubated on ice for 1 h. The sample was sonicated for 4 min (5 sec pulse, 10 sec pause, output set 5.0), and then centrifuged at 17,000×g for 30 min. The resulted pellet was washed with 50 ml Resuspension Buffer without EDTA. Then the pellet was extracted twice with 45 ml of 10 imidazole-HCl, pH 7.6, 2% sodium deoxycholate. Ninety ml of extract was mixed with 12 ml nickel-Sepharose at 4° C. for 1 h and washed 3 times with Buffer B (10 mM imidazole-HCl, pH 7.6, 0.05% Empigen BB). The resin was packed into a column and the washed with 3 c.v. of Buffer A, eluted with 10 c.v. of imidazole linear gradient (10 to 500 mM) at flow rate of 1 ml/min. Five ml fractions were collected and analyzed on SDS-PAGE. The peak fractions were pooled and dialyzed against 2 L D-PBS. OmcB-1 was soluble after removal of the sodium deoxycholate.

### Example 10

### Expression and Purification of PmpH-78

PmpH-78 was expressed from a pRRSF2 vector in a host cell in shaker flasks at 37° C. and induced by IPTG. The host cells were then harvested. Cell paste (5.5 g) was resuspened in 45 ml of Resuspension Buffer (10 mM imidazole-HCl, 20 mM sodium phosphate, 10 mM EDTA, 300 mM NaCl, pH 7.4) plus 100 ug/ml lysozyme, and incubated on ice for 1 h. The sample was sonicated for 4 min (5 sec pulse, 10 sec pause, output set 5.0), and then centrifuged at 17,000×g for 30 min. The resulted pellet was washed twice with 50 ml of 10 imidazole-HCl, pH 7.6, 2% sodium deoxycholate, once with 50 ml of 50 mM Tris-HCl, pH 8.0. Then the pellet was extracted once with 50 ml of 100 mM Tris-HCl, pH 8.0, 2 M urea and once with 50 ml of 100 mM Tris-HCl, pH 8.0, 6 M urea. Fifty ml of 6 M urea extract was mixed with 4 ml Q Sepharose FF resin at 4° C. for 1 hr. The resin was packed into a column and then washed with 5 c.v. of Buffer A (20 mM Tris-HCl, pH 8.0, 2 M urea), eluted with 20 c.v. of NaCl linear gradient (0 to 500 mM) at flow rate of 1 ml/min. Three ml fractions were collected and analyzed on SDS-PAGE. The peak fractions were pooled and dialyzed against 2 L D-PBS. PmpH-78 was soluble after removal of the denaturing agents.

### Example 11

### Expression and Purification of OmpH-1

OmpH-1 was expressed from a pRRSF2 vector in a host cell in shaker flasks at 37° C. and induced by IPTG for 3 hours. The host cells were then harvested. Cell paste (10.7 g) was resuspened in 100 ml of Resuspension Buffer (50 mM Tris-HCl, pH 7.2) plus 100 ug/ml lysozyme, and incubated on ice for 1 h. The sample was sonicated for 2 min (5 sec pulse, 10 sec pause, output set 5.0), and then centrifuged at 17,000×g for 30 min. The supernatant (90 ml) was collected and mixed with 0.9 ml of nickel-Sepharose resin for 1.5 h at 4° C. Then the resin was washed 3 times with 20 mM sodium phosphate, 0.5 M NaCl, 25 mM imidazole-HCl, pH 7.3. The resin was packed into a column and then washed with 25 mM imidazole-HCl, pH 7.5, eluted with 20 c.v. of imidazole linear gradient (25 to 500 mM). Five ml fractions were collected and analyzed on SDS-PAGE. The peak fractions were pooled and loaded to a Q Sepharose column (1.6×3 cm) which was pre-equilibrated with 20 mM Tris-HCl, 1 mM EDTA, pH 7.5. The proteins were eluted from Q Sepharose column using a linear NaCl gradient (0 to 500 mM). Fractions were analyzed on SDS-PAGE. The peak fraction were pooled and dialyzed against PBS. The purity was over 95% based on SDS-PAGE. The OmpH-1 was found to be soluble after dialysis.

### Example 12

### Western Blot Analysis of OmcB-1 and OmpH-1

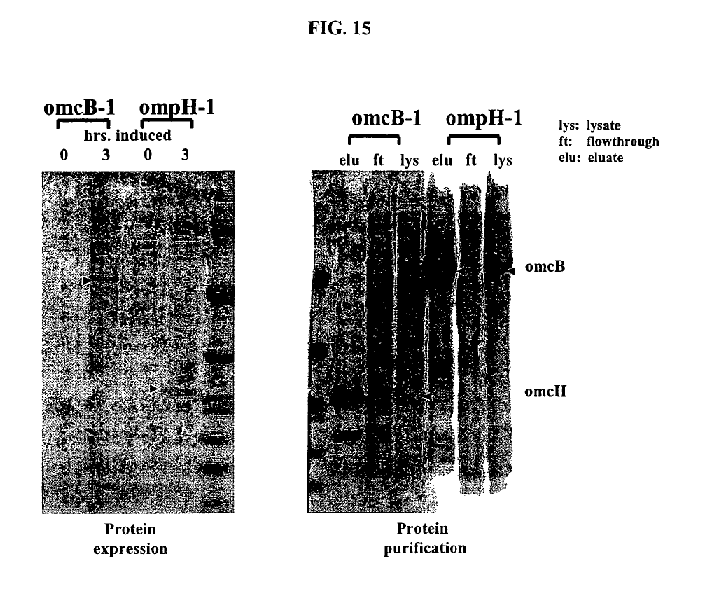

OmcB-1 and OmpH-1 proteins were grown in shake flasks at 37° C. and were induced for three hours using IPTG. Samples were taken at time 0 and 3 hours after induction. The cell cultures were spun down and pellets were resuspended in 20 mM Tris-HCl, 200 mM NaCl, and 8 M urea, pH 8.0. The cells were lysed by sonication. Supernatents were collected and incubated with 1 mL of nickel sepharose beads for a few hours at 4° C. Beads were spun down, washed, and eluted with 20 mM Tris-HCl, 200 mM NaCl, 8 M urea and 200 mM imidazole, pH 8.0. The total lysates, flowthrough and eluates were visualized on a SDS-PAGE gel, as seen in FIG. 15.

### Example 13

### Expression and Purification of PmpD-133

PmpD-133 was expressed from a pRSF2 vector in BL21 host strains with or without pLysS. The cells were grown in the same manner as described above for OmcB-1 and PmpH-1. Samples were collected after 0, 1, 2, and 3 hours of IPTG expression. Uninduced cells were used as controls. It was shown that PmpD-133 was highly expressed after three hours of induction in BL21 cells, but the expression was much depressed in BL21 cells with pLysS.

Ten grams of cell paste was resuspened in 90 ml of 50 mM Tris-HCl, pH 8.0, 10 mM EDTA, 100 ug/ml lysozyme, and incubated on ice for 1 h. The sample was sonicated for 2 min (5 sec pulse, 10 sec pause, output set 5.0), and then centrifuged at 17,000×g for 30 min. The resulted pellet was washed 3 times with 50 mM Tris-HCl, pH 8.0, 5 mM EDTA, 2% sodium deoxycholate, one time with 50 mM Tris-HCl, pH 8.0. Then the pellet was extracted once with 80 ml of 100 mM Tris-HCl, pH 8.0, 2 M urea and once with 80 ml of 100 mM Tris-HCl, pH 8.0, 6 M urea. Forty-two ml of 6 M urea extract was mixed with 4 ml Q Sepharose FF resin at 4° C. for 1 hr. The resin was packed into a column and then washed with 5 c.v. of Buffer A (20 mM Tris-HCl, pH 8.0, 2 M urea, eluted with 20 c.v. of NaCl linear gradient (0 to 500 mM) at flow rate of 0.5 ml/min. Two ml fractions were collected and analyzed on SDS-PAGE. The peak fractions were pooled and dialyzed against 2 L D-PBS. PmpD-133 was soluble after removal of the denaturing agents.

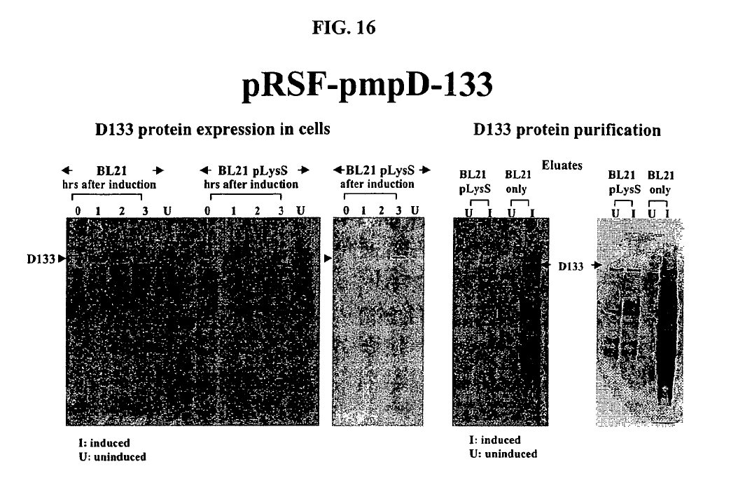

PmpD-133 proteins were also grown as described above and subsequently purified using the nickel sepharose beads as described in Example 13. Much more PmpD-133 protein was found in the eluate in BL21 cells without pLysS than that in BL21 cells with pLysS. See FIG. 16.

### Example 14

### Eluate of PmpI-63 Using Nickel Sepharose Beads

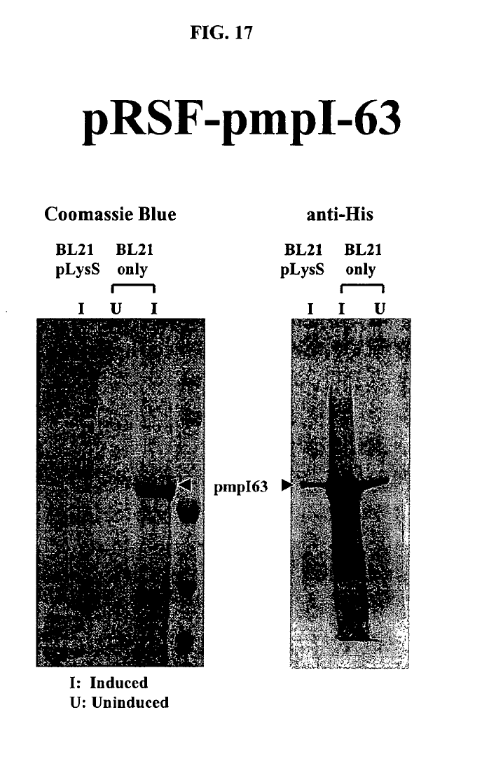

PmpI-63 protein was expressed in a pRSF2 vector in E. coli host strains BL21 with or without pLysS, and purified as described above. The eluates were run on a SDS-PAGE gel and visualized using western blotting. See FIG. 17.

### Example 15

### Animal Studies

Two murine challenge models were performed with vaccines containing purified CT110, CT84, CT57 or CT40 polypeptides as described in Examples 5 and 6: one for the vaginal challenge and the other for lung infection. The mouse lung infection model is characterized by greater susceptibility to Chlamydia infection, while the genital infection model mimics the natural infection in human. However, the mouse genital tract is not susceptible to infection by the human strain serovar E without pretreatment using the hormone progesterone. Both models evaluate the protective efficacy of the truncated CT110 proteins as vaccine candidates against Chlamydia infection.

Regardless of which challenge was used, all vaccines were diluted as necessary in PBS without calcium or phosphate prior to use. The vaccines comprised antigen (either 10 μg or 50 μg of CT110, CT84, CT57 or CT40) and adjuvant (5 μA of AB5) in deionized water.

The AB5 can be made according to U.S. Pat. No. 6,019,982. Specifically, the A and B subunits were constitutively expressed from the same vector using E. coli JM83 host cells. After expression, the cells were lysed by microfluidization and the soluble A and B subunits were collected in the supernatant fraction. The AB5 holotoxin was then purified by a two-column chromotographic method using a galactose affinity column and a gel filtration column.

The vaccines were formulated with AB5 adjuvant less than 2 hours before administration. Groups of female C31-1/HeOuJ mice (Jackson Labs) were immunized intranasally with a dose of either 10 μg or 50 μg of the purified recombinant protein vaccine. A total volume of 7.5 μL of vaccines was pipetted into each nare of anesthetized mice. Mice were vaccinated three times on Days 0, 14 and 28. Mice that were previously infected and recovered from either a vaginal or pulmonary challenge were used as positive controls in the vaginal or lung infection challenge experiments, respectively.

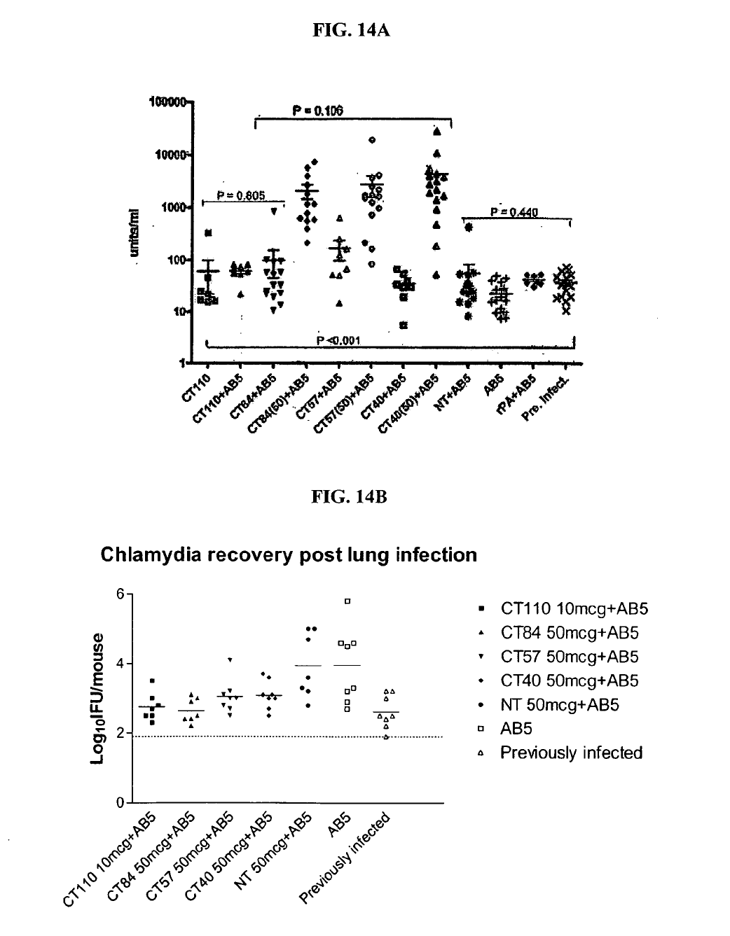

Two weeks following the final vaccination, mice were bled and lavaged vaginally using sterile PBS. Sera and vaginal lavage were stored at −20° C. until antigen-specific antibody assays were performed. The IgG anti-CT110 in serum and IgA anti-CT110 in vaginal lavage were assessed using ELISA. To determine the serum IgG or lavage IgA, microtiter plates were coated with CT110 in sodium carbonate buffer. The serum samples were then assayed against the CT110 IgG or IgA enriched mouse sera, which was assigned a value of 1000 units/ml according to previous testing. Bound antibodies were detected using peroxide-conjugated goat anti-mouse IgG or IgA antibodies, and evaluated on a spectrophotometric plate reader at 450 nm and the values geometrically averaged. FIG. 14A shows an immune response graph indicating the IgG titers of CT110, CT84, CT57 and CT40 fourteen days following the final vaccination. It was found that the immune response to CT84, CT57 and CT40 was as good as CT110, as indicated by IgG titers (FIG. 14A). When 10 μs of CT84 was used, the CT110-specific IgG titer was similar to that of 10 μg of CT110. However, when the amount of CT84, CT5, and CT40 was increased to 50 μg per dose, significantly higher IgG titer was observed for each of them.

For the vaginal challenge, two weeks after the last vaccination, the mice were administered with two doses of progesterone at an interval of 7 days. On day-21 following the last vaccination, 4×10⁶ IFU (15 μl) of C. trachomatis serovar E were delivered to the mice vaginally using a pipette. On days 3, 7, 10, 14, 21 and 30 post-challenge, vaginal samples were obtained by inserting a polyester tipped applicator into the vagina and rotated 20 times. The swab tip was then placed in SPG buffer. The sample was vortexed vigorously for 1 minute and stored at −80° C. until the assay was performed. For lung infection, two weeks post last vaccination, 4×10⁴ IFU (50 μl) of C. trachomatis serovar E were delivered to mice by a nasal route. All mice were sacrificed on day 8 post-challenge. The mouse lungs were homogenized in SPG buffer, solution spun down and the supernatant was stored at −80° C. until the assay was performed.

The protective efficacy of each recombinant protein vaccine was determined by measuring the bacterial burdens of C. trachomatis infection after the vaginal and pulmonary challenge. The bacterial burdens were monitored by inoculating the mouse fibroblast McCoy cells (ATCC) with the vaginal swab or lung homogenate supernatant samples. After infection, intracellular elementary bodies in the cells were visualized and counted using the immunohistochemical staining. The Chlamydia burdens in the samples were expressed as log₁₀ of IFU. One-way ANOVA test was used to determine the significant difference between groups. FIG. 14B shows the Chlamydia recovery following lung infection. CT84 protein clearly provided significant protective immunity against pulmonary infection when it was co-administered with AB5 via a nasal route (P<0.05 when compared to the AB5 control group, One-way ANOVA test). The efficacy induced by CT84 was similar to that of CT110 in mice. CT57 and CT40 also reduce pulmonary infection when compared to those in AB5 mock immunized mice. However, the differences induced by CT57 and CT40 were not statistically significant (P>0.05).

### Example 16

### Clinical Studies

The Chlamydia vaccines can be administered to a human subject via a mucosal route, such as through the nasal passage. Intramuscular or subcutaneous injections are also possible routes of administration. The dose levels can range from 10 to 200 μg, or 10 to 50 μg. Aluminum-based adjuvants can be used, or alternatively other adjuvants, such as MPL (Monophosphoryl Lipid A) can be used. In some embodiments, the efficacy of the new Chlamydia vaccines can be measured through controlled field studies, in which the infection rate of volunteers who have received the vaccines will be compared to that of individuals who have received the placebo. The effectiveness of the vaccines in inducing immune response in humans can be monitored by the antibody levels. However, other assays such as a cytokine Enzyme-Linked Immunospot Assay (Allen et al., Long-Lasting T Cell Responses to Biological Warfare Vaccines in Human Vaccinees. CID, volume 43, p. 1-7, 2006), or other flow cytometric assays that determine the T-cell responses can also be used since it has been shown that the cellular immune response plays a critical role in the protective immunity against the Chlamydia infection.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All patents, patent applications and publications mentioned in this specification are herein incorporated by reference to the same extent as if each individual publication or patent application was specifically and individually indicated to be incorporated by reference.

1-46. (canceled)

47. An isolated nucleic acid encoding a polypeptide comprising an amino acid sequence at least 90% identical to a reference sequence selected from the group consisting of SEQ ID NO: 2, SEQ ID NO: 11, SEQ ID NO: 13, SEQ ID NO: 19, SEQ ID NO: 21 and a combination thereof, wherein said polypeptide is soluble in the absence of denaturing agents and is recognized by an antibody that specifically binds to a polypeptide consisting of said amino acid sequence.

48. The nucleic acid of claim 47, wherein said nucleic acid encodes an amino acid sequence selected from the group consisting of SEQ ID NO: 2, SEQ ID NO: 11, SEQ ID NO: 13, SEQ ID NO: 19, SEQ ID NO: 21, and a combination thereof.

49. The nucleic acid of claim 47, wherein the coding region encoding said polypeptide is codon-optimized.

50. The nucleic acid of claim 49, wherein said coding region is codon-optimized for expression in E. coli or human.

51. The nucleic acid of claim 47, wherein said nucleic acid comprises a nucleotide sequence selected from the group consisting of SEQ ID NO: 1, SEQ ID NO: 10, SEQ ID NO: 12, SEQ ID NO: 18, SEQ ID NO: 20, and a combination thereof.

52. The nucleic acid of claim 47, wherein said nucleic acid is ligated to a heterologous nucleic acid.

53. The nucleic acid of claim 52, wherein said heterologous nucleic acid encodes a heterologous polypeptide which is fused to the polypeptide encoded by said nucleic acid.

54. The nucleic acid of claim 53, wherein said heterologous polypeptide is selected from the group consisting of a His-tag, a ubiquitin tag, a NusA tag, a chitin binding domain, ompT, ompA, pelB, DsbA, DsbC, c-myc, KSI, polyaspartic acid, (Ala-Trp-Trp-Pro), (SEQ ID NO: 10), polyphenylalanine, polycysteine, polyarginine, a B-tag, a HSP-tag, green fluorescent protein (GFP), an influenza virus hemagglutinin (HAI), a calmodulin binding protein (CBP), a galactose-binding protein, a maltose binding protein (MBP), cellulose binding domains (CBD's), dihydrofolate reductase (DHFR), glutathione-S-transferase (GST), streptococcal protein G, staphylococcal protein A, T7gene10, an avidin/streptavidin/Strep-tag, trpE, chloramphenicol acetyltransferase, lacZ (beta-galactosidase), a His-patch thioredoxin, a FLAG™ peptide, an S-tag, and a T7-tag, and a combination of two or more of said heterologous polypeptides.

55. A vector comprising the nucleic acid of claim 47.

56. The vector of claim 55, further comprising a promoter operably linked to said nucleic acid.

57. A host cell comprising the vector of claim 55.

58. A polypeptide encoded by the nucleic acid of claim 47.

59. A composition comprising the nucleic acid of claim 47 and a pharmaceutically acceptable carrier.

60. A composition comprising the polypeptide of claim 58 and a pharmaceutically acceptable carrier.

61. The composition of claim 60, further comprising an adjuvant.

62. A method to treat or prevent a Chlamydia infection in a host animal in need thereof comprising administering to said host animal an effective amount of the polypeptide of claim 59.

63. The method of claim 62, wherein said host animal is human.

64. A method of inducing an immune response against Chlamydia in a host animal in need thereof comprising administering to said host animal an effective amount of the nucleic acid of claim 47.

65. A method of inducing an immune response against Chlamydia in a host animal in need thereof comprising administering to said host animal an effective amount of the polypeptide of claim 58.

66. A method of producing a vaccine against Chlamydia comprising:
(a) isolating the polypeptide of claim 58; and
(b) adding an adjuvant to the isolated polypeptide of step (a).
